(19) United States
(12) Patent Application Publication
(10) Pub. No.: US 2008/0146277 A1
(43) Pub. Date: Jun. 19, 2008

(54) Personal healthcare assistant

(76) Inventors: Richard L. Anglin, Del Mar, CA (US); Bradley T. Tipler, San Diego, CA (US)

Correspondence Address: Giaccherini, Post Office Box 1146, Carmel Valley, CA, 93924, US

(21) Appl. No.: 11/980,167

(22) Filed: Oct. 29, 2007

Related U.S. Application Data
(63) Continuation-in-part of application No. 11/414,746, filed on Apr. 27, 2006.

(51) Int. Cl. H04M 1/00; A61B 5/00

(52) U.S. Cl. 455/556.1; 600/300

(57) ABSTRACT

Methods and apparatus for providing remote healthcare are disclosed. One embodiment of the present invention comprises a transceiver that includes a camera, a display, a speaker, a microphone and embedded remote control. This transceiver may be used at home, at work, while traveling or in any other location that offers wired or wireless access to a network, such as the Internet or a cellular telephone system. The transceiver may be used to obtain information, treatment or medical care from a Healthcare provider.

In one embodiment, the transceiver includes diagnostic and treatment software. In another alternative embodiment, the invention may also include a variety of data devices which are connected to the cellular phone over a wired or wireless connection. In one embodiment, a healthcare provider or healthcare facility may partially or jointly control the transceiver and/or a data device.

### CROSS-REFERENCE TO A RELATED PENDING PATENT APPLICATIONS & CLAIMS FOR PRIORITY

The Present patent application is a Non-Provisional, Continuation-in-Part patent application, and is related to:
Pending U.S. Non-Provisional patent application Ser. No. 11/414,746, entitled Remote Diagnostic & Treatment System, which was filed on 27 Apr. 2006; and
Pending PCT International Patent Application No. GB2007/001 519, entitled Remote Diagnostic & Treatment System, which was filed on 25 Apr. 2007.

The Applicants hereby claim the benefit of priority in accordance with 35 U.S.C. Sections 119 & 120 for any subject matter which is commonly presented in the Pending U.S. and PCT Applications referred to above, and the Present patent application.

### FEDERALLY SPONSORED RESEARCH OR DEVELOPMENT

None.

### FIELD OF THE INVENTION

One embodiment of the present invention pertains to systems, methods and apparatus for providing a Personal Healthcare Assistant. More particularly, one embodiment of the invention comprises a monitoring and/or control bi-directional communication device that may be used in a home, a workplace or in a Healthcare facility. One embodiment of the monitoring device comprises a transceiver or communication appliance, which may resemble a laptop computer. In an alternative embodiment, the transceiver includes diagnostic and treatment software. In another alternative embodiment, the invention may also include a variety of data devices that are connected to the transceiver over a wired or wireless connection. In another alternative embodiment, wireless cameras may be connected to the transceiver.

### BACKGROUND OF THE INVENTION

According to the U.S. Census Bureau, the United States saw a rapid growth in its elderly population during the 20th century. The number of Americans aged 65 and older climbed above 34.9 million in 2000, compared with 3.1 million in 1900. For the same years, the ratio of elderly Americans to the total population jumped from 1 in 25 to 1 in 8. The trend is guaranteed to continue in the coming century as the baby-boom generation grows older. Between 1990 and 2020, the population aged 65 to 74 is projected to grow seventy-four percent (74%).

This large population of elderly Americans will place great demands on Healthcare professionals and facilities. In the United States, most Healthcare is provided somewhere other than in the home. In the future, quality Healthcare will need to be provided wherever the patient might be—in the home, in the workplace or at virtually any remote location where a patient may require medical assistance, and at any time.

No currently available product offers doctors, nurses, medical technicians or other Healthcare providers a simple and versatile method and apparatus that will collect data and then provide diagnostic and treatment assistance at virtually any location over a wired or wireless connection.

The development of a system that is able to provide audio, video and data information concerning a patient or potential patient from a remote location and is able to guide a user through a diagnostic and treatment procedure at the remote location would constitute a major technological advance, and would satisfy long felt needs and aspirations in the field of Healthcare.

### SUMMARY OF THE INVENTION

One embodiment of the present invention comprises a transceiver that includes a camera, a display, a speaker, a microphone and embedded remote control. This transceiver may be used at home, at work, while traveling or in any other location that offers wired or wireless access to a network, such as the Internet or a cellular telephone system. The transceiver may be used to obtain information, treatment or medical care from a Healthcare provider.

In one embodiment, the transceiver includes diagnostic and treatment software. In another alternative embodiment, the invention may also include a variety of data devices which are connected to the cellular phone over a wired or wireless connection. In one embodiment, a Healthcare provider the Healthcare facility may partially or jointly control the transceiver and/or a data device.

An appreciation of the other aims and objectives of the present invention and a more complete and comprehensive understanding of this invention may be obtained by studying the following description of a preferred embodiment, and by referring to the accompanying drawings.

### A BRIEF DESCRIPTION OF THE DRAWINGS

FIG. 1 shows a system for collecting information in a first location and conveying that information to a second location for assessment and evaluation.

FIG. 2 shows an embodiment of a system for collecting information in a first location and conveying that information to a second location that is a call center.

FIG. 3 shows an embodiment of a system for collecting information in a first location and conveying that information to a second location that is a call center that includes additional person(s) who may provide additional information or expertise.

FIG. 4 shows a system for collecting information in a first location and conveying that information to a second location via a network.

FIG. 5 shows a system for undertaking remote triage and health status monitoring, a “virtual visit” of a patient or potential patient by a Healthcare provider.

FIG. 6 shows a system in which a person assists in collecting information in a first location and conveying that information to a second location for assessment and evaluation.

FIG. 7 shows a system for undertaking remote triage and health status monitoring, a “virtual visit” of a patient or potential patient by a Healthcare provider, which is assisted by a person in proximity to the patient or potential patient.

FIG. 8 shows an example of the Personal Healthcare Assistant for an assisted check-up of a patient in a nursing home.

FIG. 9 shows an assistant bringing a remote diagnostic means into a nursing home patient's room and placing it on a bedside table.

FIG. 10 shows an embodiment of a remote diagnostic means, a clamshell computer termed a “Virtual Visit Book™” (VVB).

FIG. 11 shows an image of a Healthcare provider on the screen of a Virtual Visit Book™ (VVB) after it has automatically connected to a call center via a cellular or Personal Communications Service (PCS) network.

FIG. 12 shows an alternative embodiment of a Virtual Visit Book™ (VVB) in which the patient sees his or her own image in addition to the image of a healthcare provider.

FIG. 13 shows a patient in a nursing home, a healthcare provider and an assistant participating in a health check-up using a Virtual Visit Book™ (VVB).

FIG. 14 shows a healthcare provider asking an assistant to remove a bandage from a patient's wound so it may be checked.

FIG. 15 shows a healthcare provider asking an assistant if she has a Visual Electronic Medical Record (VEMR™) device, and asking her to plug it into a Virtual Visit Book™ (VVB).

FIG. 16 shows a first view of a Visual Electronic Medical Record (VEMR™) device.

FIG. 17 shows a second (front) view of a Visual Electronic Medical Record (VEMR™) device.

FIG. 18 shows a third (side) view of a Visual Electronic Medical Record (VEMR™) device.

FIG. 19 shows wired and wireless connections between a Visual Electronic Medical Record (VEMR™) device and a Virtual Visit Book™ (VVB).

FIG. 20 shows a healthcare provider telling an assistant that he or she can see the image of a patient's wound as is being captured by a Visual Electronic Medical Record (VEMR™) device and transmitted by a Virtual Visit Book™ (VVB).

FIG. 21 shows a screen on a healthcare provider's diagnostic display and control means showing images of a patient and his or her wound(s).

FIG. 22 shows an embodiment of a first screen of a patient's Electronic Medical Record (EMR).

FIG. 23 shows an embodiment of a screen of a patient's Electronic Medical Record (EMR) showing the patient's personal information.

FIG. 24 shows an embodiment of a screen of a patient's Electronic Medical Record (EMR) showing the patient's medical history.

FIG. 25 shows an embodiment of a screen of a patient's Electronic Medical Record (EMR) showing whether the patient has any scheduled medical visits.

FIG. 26 shows a Visual Electronic Medical Record (VEMR™) device connected to a computer terminal.

FIG. 27 shows wired and wireless connections between a Visual Electronic Medical Record (VEMR™) device and a computer terminal.

FIG. 28 shows alternative embodiments of a Virtual Visit Book™ (VVB).

FIG. 29 shows a Visual Electronic Medical Record (VEMR™) device connected to alternative embodiments of a Virtual Visit Book™ (VVB) via a wired connection.

FIG. 30 shows a Visual Electronic Medical Record (VEMR™) device connected to alternative embodiments of a Virtual Visit Book™ (VVB) via a wireless connection.

FIG. 31 shows a specific embodiment of a Virtual Visit Book™ (VVB), a One Laptop Per Child (OLPC) XO laptop computer.

FIG. 32 shows wireless mesh networking between multiple One Laptop Per Child (OLPC) XO laptop computers as embodiments of a Virtual Visit Book™ (VVB).

FIG. 33 shows Visual Electronic Medical Record (VEMR™) devices connected to a wireless mesh network between multiple One Laptop Per Child (OLPC) XO laptop computers as embodiments of a Virtual Visit Book™ (VVB).

FIG. 34 shows a first preferred embodiment of a functional block diagram of the diagnostic, display and control software application deployed on a remote diagnostic means.

FIG. 35 shows a first preferred embodiment of a functional block diagram of the diagnostic, display and control software application deployed on a diagnostic, display and control means.

FIG. 36 shows a first preferred embodiment of a functional block diagram of the diagnostic, display and control software application deployed on a Visual Electronic Medical Record (VEMR™) device.

FIG. 37 shows a first preferred embodiment of a functional block diagram of the diagnostic, display and control software application deployed on a diagnostic, display and control means to control a Visual Electronic Medical Record (VEMR™) device.

FIG. 38 shows the disclosed invention in which one or more data devices are connected to device or terminal.

FIG. 39 shows a preferred embodiment of the disclosed invention in which one or more data devices are connected to a device or terminal via a wireless connection.

FIG. 40 shows a preferred embodiment of the disclosed invention in which one or more data devices are connected to a device or terminal via a wired connection.

FIG. 41 shows a preferred embodiment of the disclosed invention in which one or more data devices are connected to a Virtual Visit Book™ (VVB) via a wired connection.

FIG. 42 shows a preferred embodiment of the disclosed invention in which one or more data devices are connected to a Virtual Visit Book™ (VVB) via a wireless connection.

FIG. 43 shows the functional block diagram for a connection interface device.

FIG. 44 shows a data device, a digital thermometer.

FIG. 45 shows a first embodiment of a functional block diagram of embedded software that enables remote functional control of data devices for the remote diagnostic means as well as a software application that enables remote functional control of the data devices for the remote diagnostic means.

FIG. 46 shows a pop-up window for the location of the temperature reading on the body.

FIG. 47 shows a screen displayed on a diagnostic, display and control means used by a Healthcare provider showing the temperature.

FIG. 48 shows the temperature reading fed to the connection interface device for connection to the remote diagnostic means.

FIG. 49 shows a data device, a stethoscope or high fidelity microphone.

FIG. 50 shows a second embodiment of a functional block diagram of embedded software that enables remote functional control of data devices for the remote diagnostic means as well as a software application that enables remote functional control of the data devices for the remote diagnostic means.

FIG. 51 shows a screen displayed on a diagnostic, display and control means used by a Healthcare provider showing the pulse.

FIG. 52 shows a transducer converting the stethoscope sound into electrical signals that are fed to the connection interface device for connection to the remote diagnostic means.

FIG. 53 shows a data device, a scale.

FIG. 54 shows a third embodiment of a functional block diagram of embedded software that enables remote functional control of data devices for the remote diagnostic means as well as a software application that enables remote functional control of the data devices for the remote diagnostic means.

FIG. 55 shows a screen displayed on a diagnostic, display and control means used by a Healthcare provider showing the weight.

FIG. 56 shows the weight reading fed to the connection interface device for connection to the remote diagnostic means.

FIG. 57 shows a data device, a blood pressure cuff.

FIG. 58 shows a fourth embodiment of a functional block diagram of embedded software that enables remote functional control of data devices for the remote diagnostic means as well as a software application that enables remote functional control of the data devices for the remote diagnostic means.

FIG. 59 shows a screen displayed on a diagnostic, display and control means used by a Healthcare provider showing the blood pressure and pulse readings.

FIG. 60 shows the pulse and blood pressure readings fed to the connection interface device for connection to the remote diagnostic means.

FIG. 61 shows a data device, an oximeter.

FIG. 62 shows a fifth embodiment of a functional block diagram of embedded software that enables remote functional control of data devices for the remote diagnostic means as well as a software application that enables remote functional control of the data devices for the remote diagnostic means.

FIG. 63 shows a screen displayed on a diagnostic, display and control means used by a Healthcare provider showing the oximeter and pulse readings.

FIG. 64 shows the pulse and oximeter readings fed to the connection interface device for connection to the remote diagnostic means.

FIG. 65 shows a data device, an electrocardiogram (EKG or ECG).

FIG. 66 shows a sixth embodiment of a functional block diagram of embedded software that enables remote functional control of data devices for the remote diagnostic means as well as a software application that enables remote functional control of the data devices for the remote diagnostic means.

FIG. 67 shows a screen displayed on a diagnostic, display and control means used by a Healthcare provider showing the electrocardiogram and pulse readings.

FIG. 68 shows the pulse and electrocardiogram readings fed to the connection interface device for connection to the remote diagnostic means.

FIG. 69 shows a data device, a glucose meter.

FIG. 70 shows a seventh embodiment of a functional block diagram of embedded software that enables remote functional control of data devices for the remote diagnostic means as well as a software application that enables remote functional control of the data devices for the remote diagnostic means.

FIG. 71 shows a screen displayed on a diagnostic, display and control means used by a Healthcare provider showing the glucose reading.

FIG. 72 shows the glucose reading fed to the connection interface device for connection to the remote diagnostic means.

FIG. 73 shows a data device, an otoscope.

FIG. 74 shows an eighth embodiment of a functional block diagram of embedded software that enables remote functional control of data devices for the 1 remote diagnostic means as well as a software application that enables remote functional control of the data devices for the remote diagnostic means.

FIG. 75 shows a screen displayed on a diagnostic, display and control means used by a Healthcare provider showing the otoscope reading.

FIG. 76 shows the otoscope picture fed to the connection interface device for connection to the remote diagnostic means.

FIG. 77 shows a data device, an ultrasound device.

FIG. 78 shows a ninth embodiment of a functional block diagram of embedded software that enables remote functional control of data devices for the remote diagnostic means as well as a software application that enables remote functional control of the data devices for the remote diagnostic means.

FIG. 79 shows a screen displayed on a diagnostic, display and control means used by a Healthcare provider showing the ultrasound.

FIG. 80 shows the ultrasound readings fed to the connection interface device for connection to the remote diagnostic means.

FIG. 81 shows a data device, a spirometer.

FIG. 82 shows a tenth embodiment of a functional block diagram of embedded software that enables remote functional control of data devices for the remote diagnostic means as well as a software application that enables remote functional control of the data devices for the remote diagnostic means.

FIG. 83 shows a screen displayed on a diagnostic, display and control means used by a Healthcare provider showing the spirometer readings.

FIG. 84 shows the spirometer readings fed to the connection interface device for connection to the remote diagnostic means.

FIG. 85 shows a data device, a digital camera.

FIG. 86 shows a data device, a digital video camera.

FIG. 87 shows an eleventh embodiment of a functional block diagram of embedded software that enables remote functional control of data devices for the remote diagnostic means as well as a software application that enables remote functional control of the data devices for the remote diagnostic means.

FIG. 88 shows a screen displayed on a diagnostic, display and control means used by a Healthcare provider showing the external camera image.

FIG. 89 shows the image from an external camera fed to the connection interface device for connection to the remote diagnostic means.

FIG. 90 shows an external camera observing a patient or potential patient utilizing a remote diagnostic means, including data devices.

FIG. 91 shows an alternative embodiment of the remote diagnostic means as a Personal Computer (PC) equipped with a camera.

FIG. 92 shows an alternative embodiment of the remote diagnostic means as a laptop Personal Computer (PC) equipped with a camera.

FIG. 93 shows data devices connected to a personal computer via an Universal Serial Bus (USB) connection.

FIG. 94 shows data devices connected to a personal computer via an Institute of Electrical and Electronics Engineers (IEEE) 1394 FireWire connection.

FIG. 95 shows data devices connected to a personal computer using a Bluetooth®, wireless fidelity (WiFi), Ultra Wide Band (UWB), ZigBee™ and/or World Interoperability for Microwave Access (WiMax) connection.

FIG. 96 shows data devices connected to a cellular or Personal Communications Service (PCS) wireless phone using a Bluetooth®, wireless fidelity (WiFi), Ultra Wide Band (UWB), ZigBee™ and/or World Interoperability for Microwave Access (WiMax) connection.

FIG. 97 shows data devices connected to a cellular or Personal Communications Service (PCS) wireless phone via an Universal Serial Bus (USB) connection.

FIG. 98 shows data devices connected to a cellular or Personal Communications Service (PCS) wireless phone via an Institute of Electrical and Electronics Engineers (IEEE) 1394 FireWire connection.

FIG. 99 shows data devices connected to an One Laptop Per Child (OLPC) XO laptop computer via an Universal Serial Bus (USB) connection.

FIG. 100 shows data devices connected to One Laptop Per Child (OLPC) XO laptop computer via an Institute of Electrical and Electronics Engineers (IEEE) 1394 FireWire connection.

FIG. 101 shows data devices connected to an One Laptop Per Child (OLPC) XO laptop computer using a Bluetooth®, wireless fidelity (WiFi), Ultra Wide Band (UWB), ZigBee™ and/or World Interoperability for Microwave Access (WiMax) connection.

FIG. 102 shows an embodiment of the remote diagnostic means, a Virtual Visit Book™ (VVB), deployed in an assisted living environment for elderly persons.

FIG. 103 shows a portable embodiment of the remote diagnostic means, a Virtual Visit Book™ (VVB).

FIG. 104 shows a mobile embodiment of the remote diagnostic means, a Virtual Visit Book™ (VVB) that includes a Global Positioning System (GPS) receiver, deployed in an automobile.

FIG. 105 shows a screen displayed on a diagnostic, display and control means used by a healthcare provider showing the Global Positioning System (GPS) location of the patient.

FIG. 106 shows an embodiment of the remote diagnostic means, a Virtual Visit Book™ (VVB), used in a post traumatic stress syndrome counseling session.

FIG. 107 shows an embodiment of the remote diagnostic means, a Virtual Visit Book™ (VVB) and a Virtual Electronic Medical Record (VEMR™) device, used for assessing the fit of a prosthesis.

FIG. 108 shows the disclosed invention in which the healthcare provider uses a Virtual Visit Book™ (VVB) at home as the diagnostic, display and control means.

FIG. 109 shows the disclosed invention in which the healthcare provider uses a laptop Personal Computer (PC) at home as the diagnostic, display and control means.

FIG. 110 shows the disclosed invention in which the healthcare provider uses a cellular or Personal Communications Service (PCS) wireless phone as the diagnostic, display and control means.

FIG. 111 shows the disclosed invention, a Virtual Visit Book™ (VVB), used in a physical rehabilitation or athletic coaching application in a fixed setting.

FIG. 112 shows the disclosed invention used in a physical rehabilitation or athletic coaching application on the move.

FIG. 113 shows a Bluetooth, wireless fidelity (WiFI), Ultra Wide Band (UWB), ZigBee™ or World Interoperability for Microwave Access (WiMax)-enabled heart rate monitor.

FIG. 114 shows a patient in a remote location, here a ship, using a satellite phone and a Virtual Electronic Medical Record (VEMR™) device as a diagnostic means.

FIG. 115 shows a patient in a remote location, here an airplane, using a satellite phone and a Virtual Electronic Medical Record (VEMR™) device as a diagnostic means.

FIG. 116 shows an embodiment of the disclosed invention, a Virtual Visit Book™ (VVB) and a Virtual Electronic Medical Record (VEMR™) device, used by a veterinarian to undertake remote triage and health status monitoring of an animal.

FIG. 117 shows a fire investigator using a chemical sniffer attached to a Virtual Visit Book™ (VVB) to get data about the potential causes of a fire that are transmitted to a laboratory analyst for assessment.

FIG. 118 shows a policeman uses a remote fingerprint device attached to a Virtual Visit Book™ (VVB) to fingerprint a suspect, which fingerprint is transmitted to an analyst for review and matching to fingerprint databases.

FIG. 119 shows an engineer using a soil sampling device attached to a Virtual Visit Book™ (VVB), characteristics of which are transmitted to an laboratory analyst for assessment.

FIG. 120 shows a fire investigator is using a chemical sniffer attached to a walkie-talkie phone who transmits that data in real time to a fire fighter actively fighting the fire nearby.

FIG. 121 shows a Virtual Electronic Medical Record (VEMR™) device and data devices connected to connected to a Virtual Visit Book™ (VVB) with a dial-up modem to connect to the Public Switched Telephone Network (PSTN).

FIG. 122 shows a barrel attached to the front of a Virtual Electronic Medical Record (VEMR™) device.

FIG. 123 shows a hood attached to the front of a Virtual Electronic Medical Record (VEMR™) device.

FIG. 124 shows a cone attached to the front of a Virtual Electronic Medical Record (VEMR™) device.

FIG. 125 shows an adjustable distance scale attached to the front of a Virtual Electronic Medical Record (VEMR™) device.

FIG. 126 shows a ruler inside a barrel attached to the front of a Virtual Electronic Medical Record (VEMR™) device.

FIG. 127 shows a ruler inside a hood attached to the front of a Virtual Electronic Medical Record (VEMR™) device.

FIG. 128 shows a ruler attached to an adjustable distance scale attached to the front of a Virtual Electronic Medical Record (VEMR™) device.

FIG. 129 shows a distance sensor for determining the distance to a wound or other injury from the front of a Virtual Electronic Medical Record (VEMR™) device.

FIG. 130 shows a distance sensor on the front of a Virtual Electronic Medical Record (VEMR™) device.

FIG. 131 shows a grid on the image from a Virtual Electronic Medical Record (VEMR™) device.

FIG. 132 shows a stylus used to annotate the image from a Virtual Electronic Medical Record (VEMR™) device.

FIG. 133 shows a knob to manually focus a Virtual Electronic Medical Record (VEMR™) device.

FIG. 134 shows the steps a patient or potential patient must take to receive remote diagnostic and health status monitoring services.

FIG. 135 shows the steps a patient or potential patient must take to receive remote diagnostic and health status monitoring services using a cellular, Personal Communications Service (PCS) or Wireless Fidelity (WiFi) wireless phone.

FIG. 136 shows a first embodiment of a functional block diagram of an institutional structure or business model under which remote diagnostic and health status monitoring services may be provided.

FIG. 137 shows a second embodiment of a functional block diagram of an institutional structure or business model under which remote diagnostic and health status monitoring services may be provided.

FIG. 138 shows a third embodiment of a functional block diagram of an institutional structure or business model under which remote diagnostic and health status monitoring services may be provided.

FIG. 139 shows the disclosed invention used by a health care provider to consult with another health care provider.

FIG. 140 shows a preferred embodiment of the disclosed invention with wireless cameras deployed to observe the scene of an accident or emergency, including one on an Emergency Medical Technician's shoulder.

FIG. 141 shows wireless cameras deployed inside an ambulance or other emergency vehicle.

FIG. 142 shows two Emergency Medical Technicians collecting medical condition data from a victim.

FIG. 143 shows an alternative medical practitioner terminal display showing wireless camera images from an ambulance or other emergency vehicle.

FIG. 144 shows a medical practitioner terminal display allowing the medical practitioner to choose a wireless camera or a Visual Electronic Medical Record (VEMR™) device to control.

FIG. 145 shows an embodiment of a functional block diagram of embedded software that enables remote functional control of a wireless camera.

FIG. 146 shows a three-way video consultation between an Emergency Medical Technician, a medical practitioner and a specialist medical practitioner.

FIG. 147 shows an alternative embodiment of the disclosed invention in which Virtual Visit Books™ (VVBs) are used to observe a victim as well as to collect and transmit medical condition data.

FIG. 148 shows an Emergency Medical Technician communicating directly with a remote medical practitioner using a Virtual Visit Book™ (VVB) to transmit video and medical condition data.

FIG. 149 shows a trainee being observed by a remotely located trainer as part of an initial or recurrent training program.

FIG. 150 shows a person being observed by a remotely located mentor.

### A DETAILED DESCRIPTION OF PREFERRED & ALTERNATIVE EMBODIMENTS

### I. Overview of the Invention

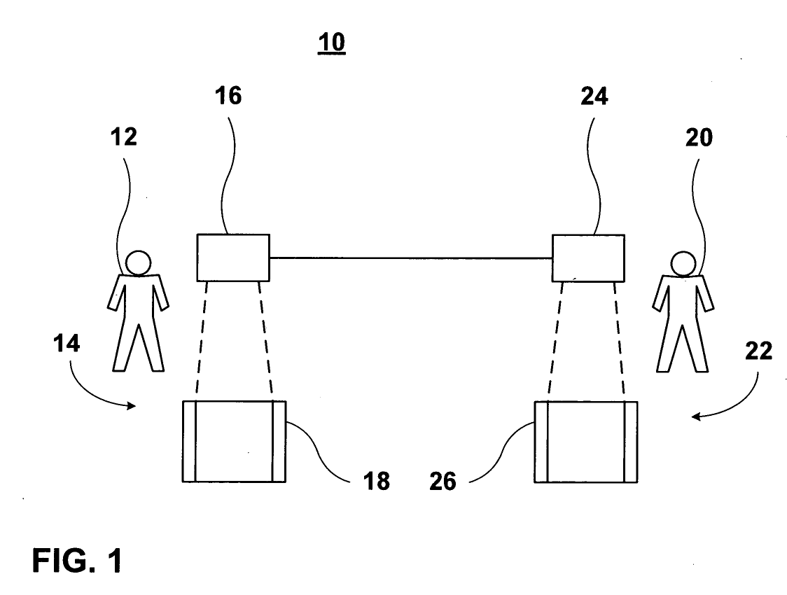

FIG. 1 depicts a Personal Healthcare Assistant 10. In the embodiment shown in FIG. 1, a first person 12 in location 14 has a device or terminal 16, including embedded software 18. The device 16 may be used to collect information that is then conveyed to a second person 20 in a second location 22. The second person 20 uses another device or terminal 24 that includes software 26. The second person 20 may use his or her device 24 and software 26 to provide information and assistance to the first person 12. Additionally, the software 26 in device or terminal 24 may be used to control some or all of the features of the device or terminal 16 via its embedded software 18.

The devices 16, 24 function as transceivers that send and/or receive some combination of audio signals, video signals and data. The transceiver may convey this combination of audio signals, video signals and/or data using a wired or wireless connection. The transceiver may communicate wirelessly using a cellular telephone system, WiFi, Wi-MAX or other wireless system. In this Specification and in the Claims that follow, the term “transceiver” encompasses any apparatus or system, including hardware or software, that emits or receives a signal that may be used to convey information, data or some other form of intelligence. In an alternative embodiment, the transceiver may convey this combination of signals over a network like the Internet. This embodiment may utilize a Web browser and/or a website.

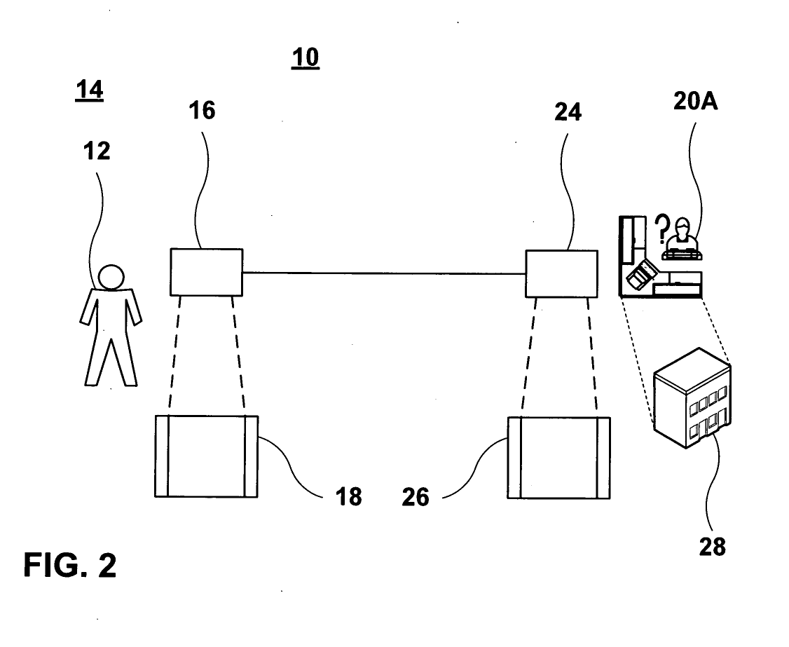

In one specific embodiment of the invention shown in FIG. 2, the first person or user 12 employs his or her first device 16 to contact a call center 28. When used in this Specification and in the Claims that follow, the term “call center” 28 encompasses any facility, establishment or provision for receiving a call, request, message or signal from the first user 12. The call center 28 may comprise a building, facility, place or site staffed by a plurality of operators, technicians, advisors or other personnel. The call center 28 may include any number of live operators 20A, including a single person 20B working in an office, at home or in any other location. The single person 20B may be a specialist or other person with particular knowledge or expertise. In an alternative embodiment, the call center 28 may function without any live human assistance, and may rely on software running on a server, voice recognition equipment, recordings, and/or other automated systems.

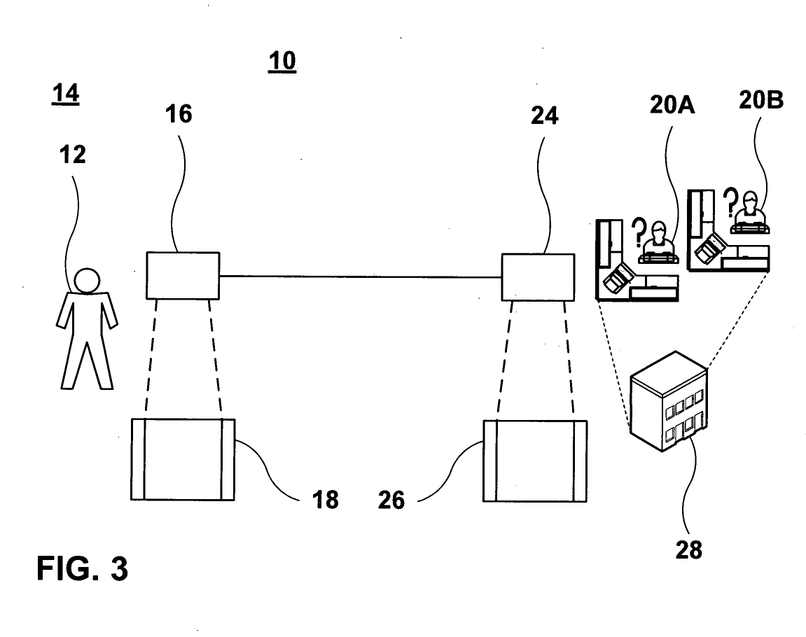

Similarly, the term “second person” 20 or any other specific individual denotes any combination of persons or automated systems at the other end of the call from the point of view of the first person 12, and may essentially be functionally equivalent to the term “call center” 28. FIG. 3 shows an additional person 20B who may provide additional information or expertise in responding to a call, request, message or signal from the first user 12. This additional person 20B may be physically located at a “brick and mortar” call center 28 or be functioning in a distributed mode.

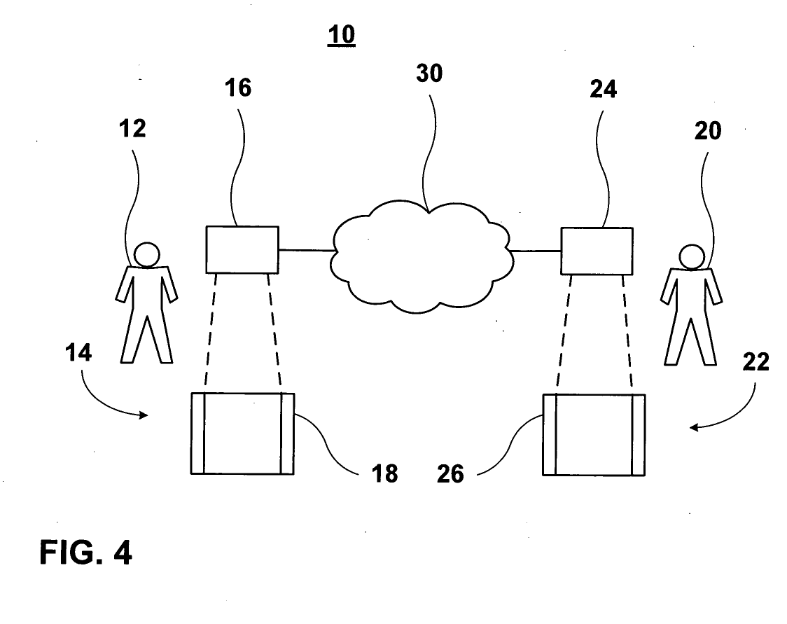

In general, the present invention encompasses any plurality of devices or terminals 16, 24 that are used in combination using a wired or wireless connection over a network 30 as shown in FIG. 4. The network 30 may comprise any combination of wired or wireless connections, including a direct device-to-device link. The first terminal or device 16 is employed to collect, measure, record or otherwise process, store or receive data or information that is then conveyed to another terminal or device 24. The first device 16 has software 18 that enables a user 12 to collect data with the first device 16. The second device 24 has software 26 which enables some form of analysis, examination or response from the user 20 of the second terminal or device 24 back to the user 12 of the first device 16. The software 26 in the second device 24 also has the ability to remotely control some or all of the functions of the first device 16.

The present invention also encompasses any plurality of devices 16 that are used cooperatively to gather information in one place 14, and then use, store, assay, process the data or formulate a response to the data in another place 22. The two locations 14 and 22 may generally be separated by any distance. In addition, the invention provides for the remote direction, monitoring or guidance of the first device 16 by the user 20 of the second device 24. In an alternative embodiment, the user 20 of the second device 24 controls, partially or fully, the operation of the first device 16. In another embodiment, an automated system may control the operation of the first device 16.

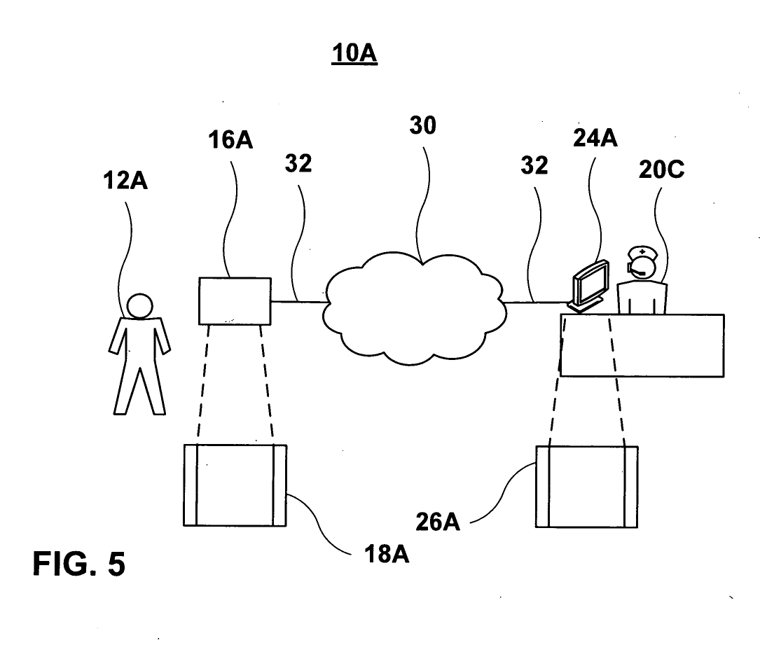

In one particular embodiment of the Personal Healthcare Assistant 10A, shown in FIG. 5, the “Virtual Visit System™,” includes a diagnostic means 16A for collecting data that has diagnostic means software 18A embedded in the diagnostic means 16A that enables remote function control of the diagnostic means 16A. The specific embodiment of the remote diagnostic means 16A collects information about a patient 12A. The diagnostic means 16A conveys data over a connection 32 to a network 30 to a diagnostic, display and control means 24A which runs one or more software application(s) 26A. In this embodiment, the diagnostic, display and control means 24A and software 26A is used by a healthcare provider 20C. In this embodiment, the term “diagnostic” refers to the process of determining or identifying an illness, disease, injury or sickness or other physical or mental condition as a precursor to furnishing an opinion, advice or suggested course of treatment.

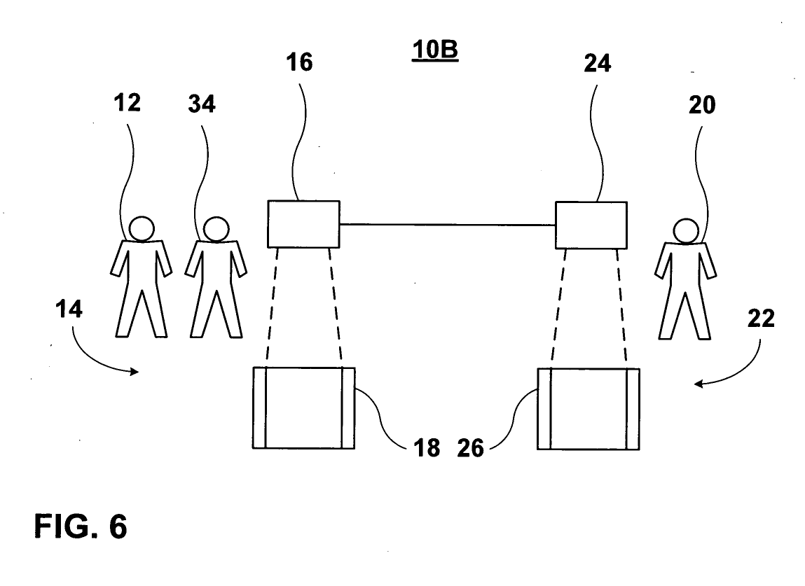

In almost every circumstance there will be a third person 34, an “assistant” or “aider,” someone in close physical proximity to the first person 12, who will provide assistance in collecting information about the first person 12 and conveying it to a second person, as shown in FIG. 6. In most circumstances the assistant 34 is using, operating or facilitating the operation of the diagnostic means 16.

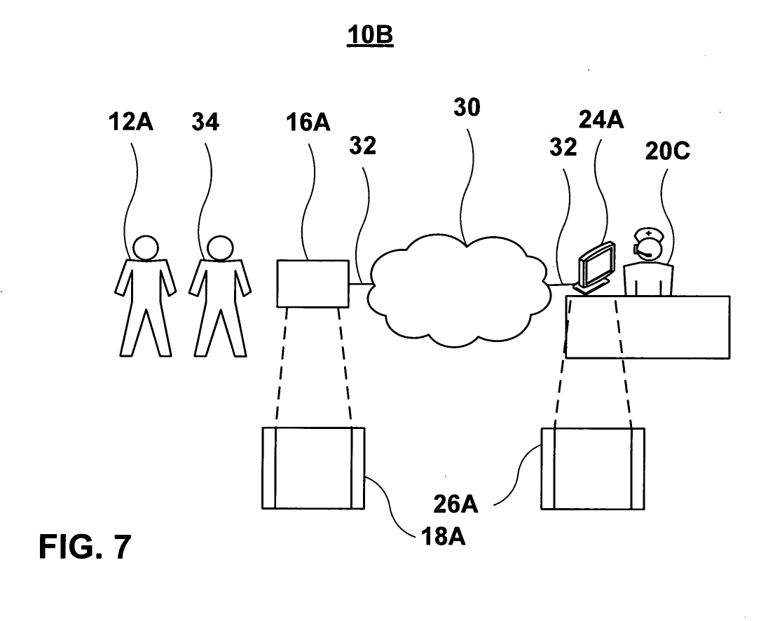

In one particular embodiment of the Personal Healthcare Assistant 10B, which is shown in FIG. 7, the Virtual Visit System™, includes a diagnostic means 16A used by an aider 34 for collecting data that has diagnostic means software 18A embedded in the diagnostic means 16A that enables remote function control of the diagnostic means 16A. The specific embodiment of the remote diagnostic means 16A collects information about a patient or potential patient 12A. The diagnostic means 16A conveys data over a connection 32 to a network 30 to a diagnostic, display and control means 24A which runs one or more software application(s) 26A. In this embodiment, the diagnostic, display and control means 24A and software 26A is used by a healthcare provider 20C. In this embodiment, the term “diagnostic” refers to the process of determining or identifying an illness, disease, injury or sickness or other physical or mental condition as a precursor to furnishing an opinion, advice or suggested course of treatment.

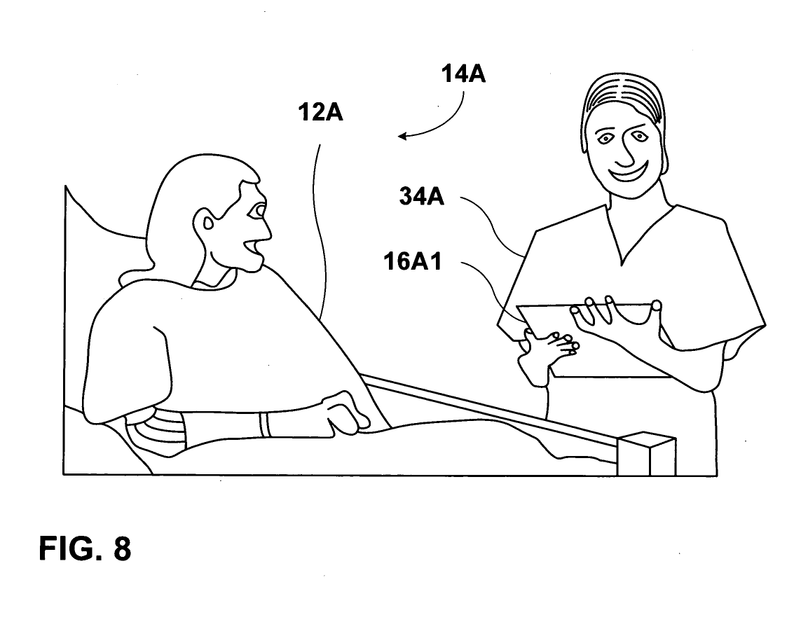

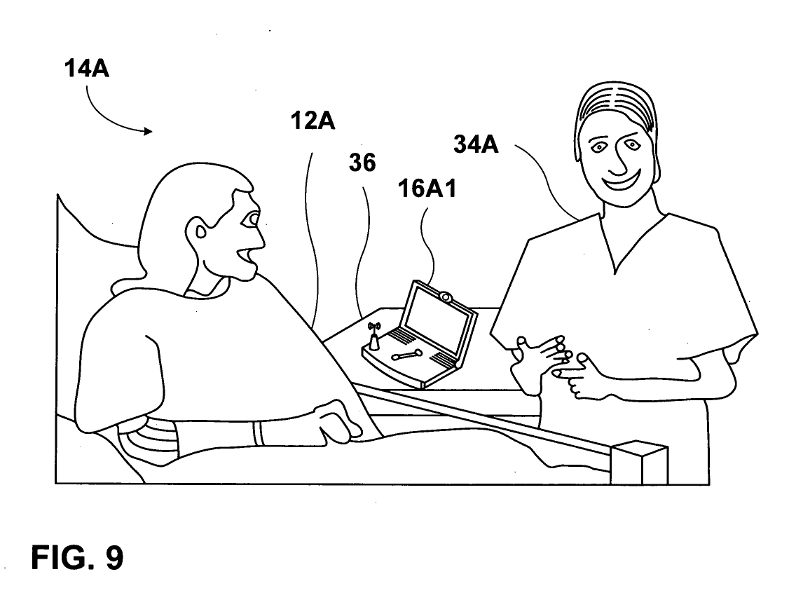

An example of the Personal Healthcare Assistant 10B is shown in FIG. 8. Here a worker 34A, perhaps a nurse but more likely a lay person, at a nursing home 14A brings a remote diagnostic means 16A1 into the patient's 12A room for his or her checkup. In the instant embodiment the worker 34A places the remote diagnostic means 16A1 on a bedside table 36, and opens it up. See FIG. 9.

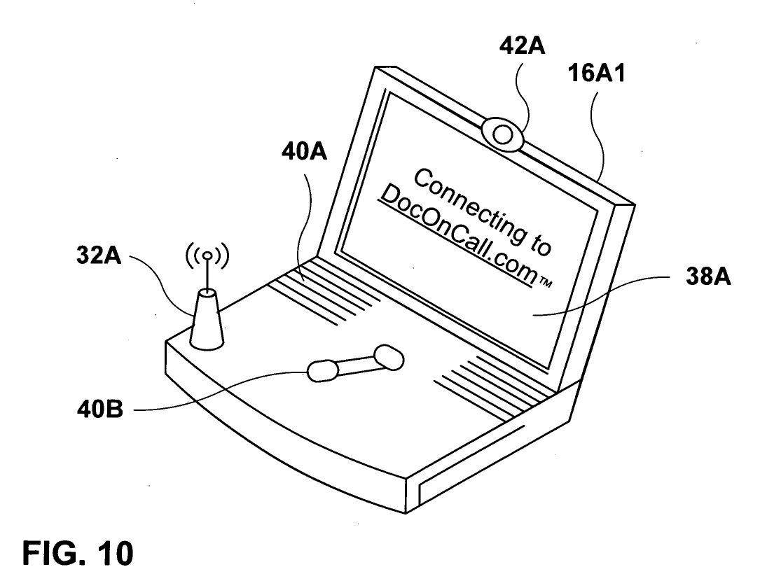

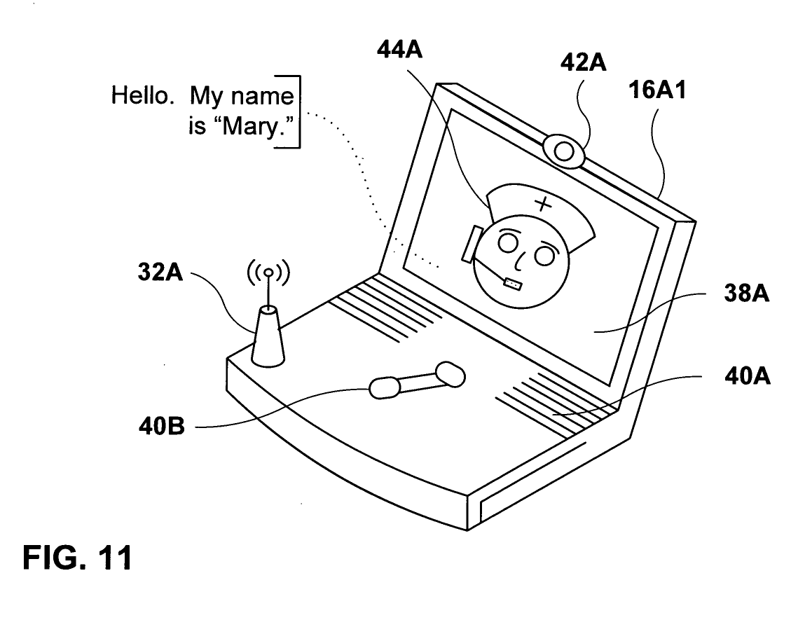

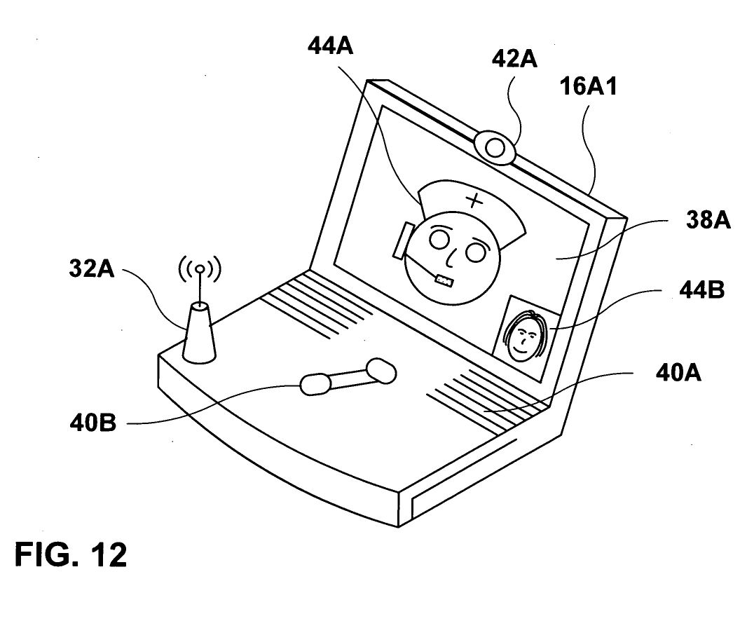

A particular embodiment of a remote diagnostic means 16A1 is shown in FIG. 10. This embodiment of a remote diagnostic means 16A1 is a battery-operated personal computer in a “clamshell” design, here termed the “Virtual Visit Book™” (VVB). It has no cords to plug in, no on/off switch, no mouse and no keyboard. It has a display screen 38A, built-in speakers 40A and a built-in microphone 40B. In an alternative embodiment the microphone and speakers are combined in a speaker-phone 40. It also has a built-in camera 42A. When the Virtual Visit Book™ (VVB) 16A1 is opened it automatically wirelessly connects 32 to a call center 28. In a first embodiment the connection 32A is via a cellular or Personal Communications Service (PCS) network 30A. The VVB 16A1 automatically sets up a “Visual Virtual Visit” without any action by the assistant 34A or the patient 12A, as shown in FIG. 11. An image 44A of a healthcare provider 20C appears on the screen 38A of the VVB 16A1. In an alternative embodiment of the VVB 16A1, the patient 12A sees his or her own image 44B in addition to the image 44A of the healthcare provider 20C, as shown in FIG. 12.

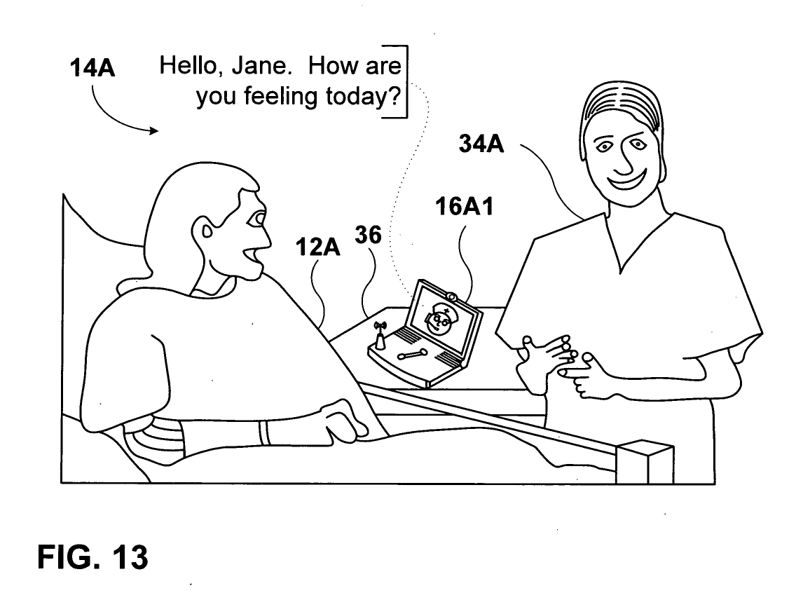

FIG. 13 shows a patient 12A in a nursing home 14A, a healthcare provider 20C in a call center 28, and an assistant 34A participating in a health check-up, or health status monitoring.

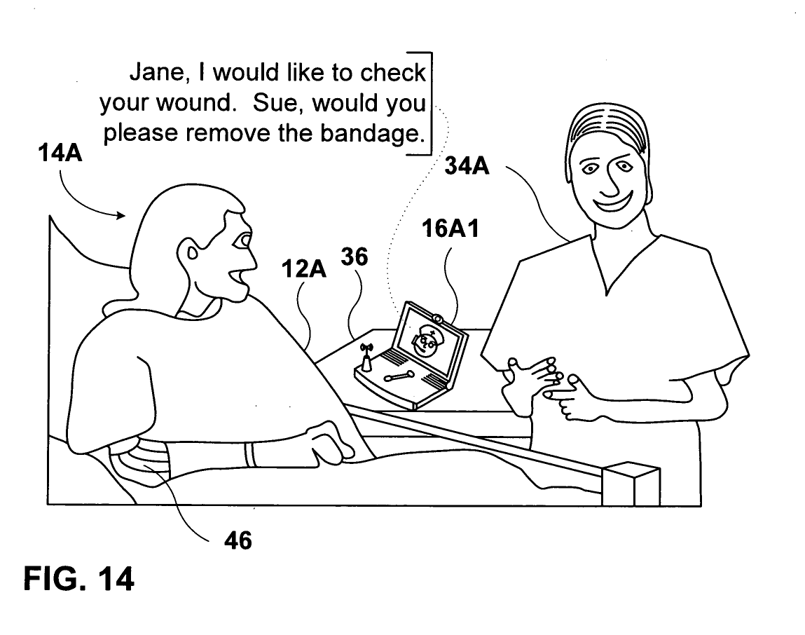

In FIG. 14 the healthcare provider 20C tells the patient 12A that he or she wants to check on the condition of a wound and asks the assistant 34A to remove the bandage 46.

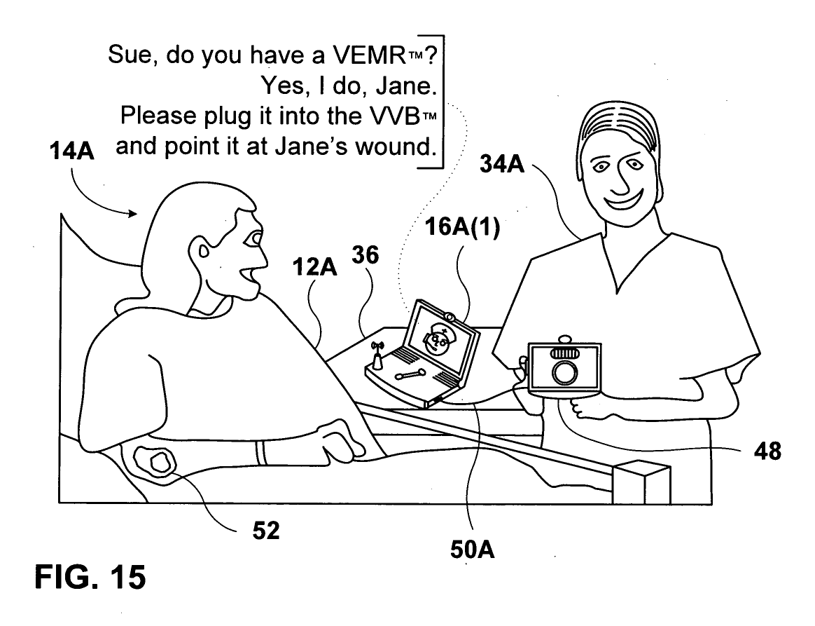

In FIG. 15 the healthcare provider 20C asks the assistant 34A if she has a Visual Electronic Medical Record (VEMR™) device 48 available. The assistant 34A says, “yes.” The healthcare provider 20C asks the assistant 34A to connect the VEMR 48 to the VVB 16A1 via its Universal Serial Bus (USB) connection 50A. In the instant embodiment the VEMR 48 is powered via the USB connection 50A. The healthcare provider 20C then directs the assistant 34A to turn the VEMR 48 “on” and point the VEMR 48 at the patient's 12A wound 52.

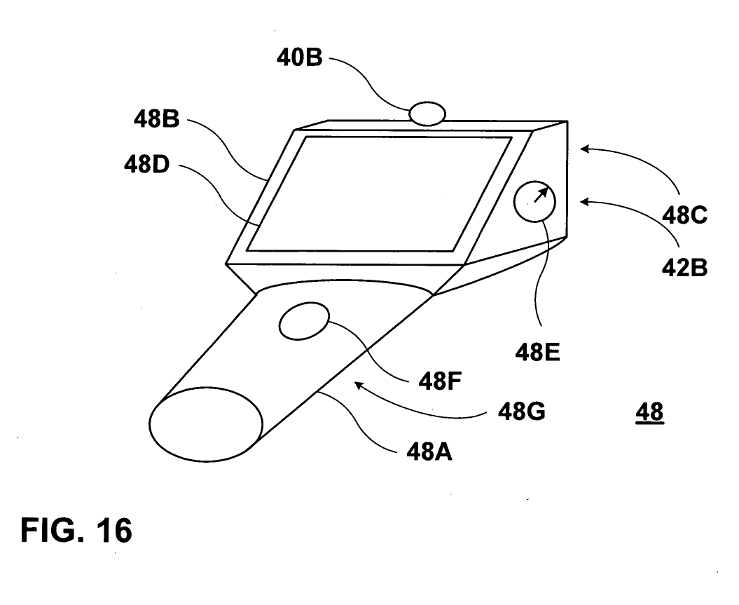

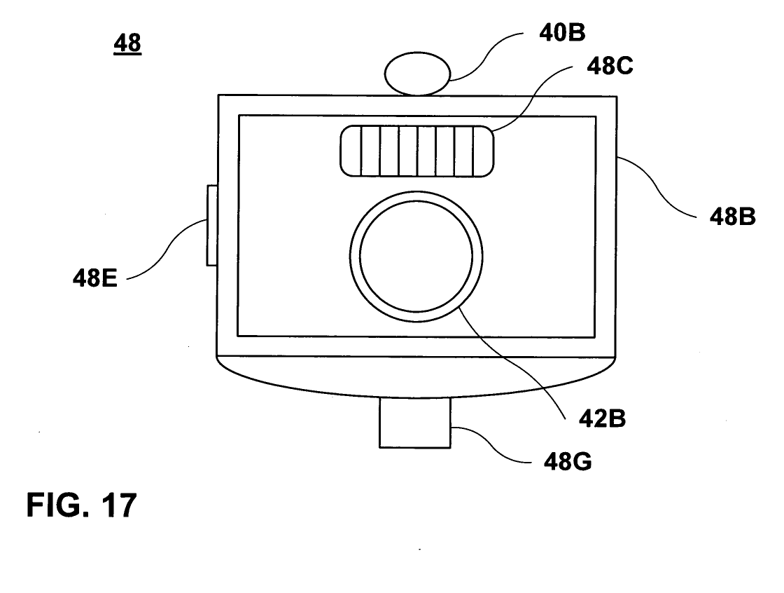

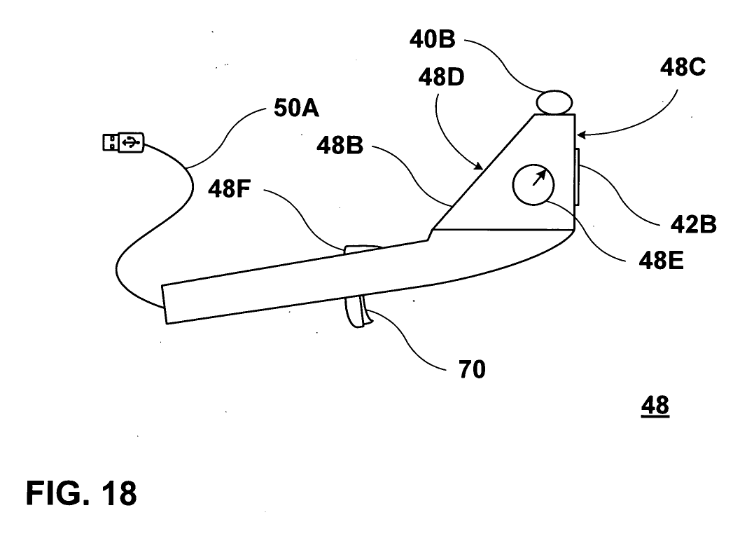

FIG. 16 shows a view of a preferred embodiment of a Visual Electronic Medical Record (VEMR™) device 48, a hand-held device that includes a handle 48A, a housing 48B containing a high resolution digital camera 42B capable of medical quality imaging, both video and image, an illumination source 48C aligned with the camera 42B field of view, a viewing screen 48D and a built-in microphone 40B. The device also includes a mode of operation selector 48E, an “on-off” switch 48F and a “trigger” 48G for capturing images and videos. FIGS. 17 and 18 show a front view and a side view respectively of a preferred embodiment of a VEMR device 48. FIG. 18 also shows an embodiment of a VEMR device with a USB connection 50A.

Alternative embodiments of the VEMR device may or may not include all of the features shown in FIGS. 16 through 18. The essential features of any embodiment are a digital camera 42B and an illumination source 48C.

There are numbers of technologies that may be used for the connection 50 between a Virtual Visit Book™(VVB) 16A1 and a Visual Electronic Medical Record (VEMR™) device 48, both wired and wireless.

A first embodiment of a wired connection between a VEMR 48 and a device or terminal 16 is Universal Serial Bus (USB) 50A, an external bus standard that supports data transfer rates of twelve megabits per second (12 Mbps) for up to one hundred twenty-seven peripheral devices.

A second embodiment of a wired connection between a VEMR 48 and a device or terminal 16 is FireWire 50B, IEEE 1394, High Performance Serial Bus. FireWire provides a single plug-and-socket connection on which up to sixty-three devices can be attached with data transfer speeds up to four hundred megabits per second (400 Mbps).

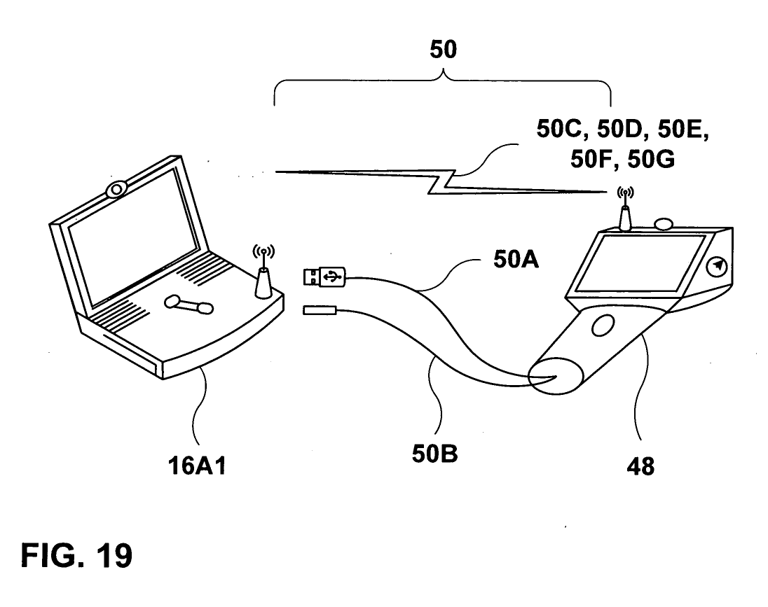

Wired connections 50A, 50B between a VEMR 48 and a specific device or terminal 16, a VVB 16A1, are shown in FIG. 19.

A first embodiment of a wireless connection between a VEMR 48 and a device or terminal 16 is Bluetooth® 50C, a low-power radio communications to wirelessly link phones, computers and other network devices over short distances. Wireless signals transmitted with Bluetooth cover short distances, typically up to thirty feet (30 ft) or ten meters (10 m).

“WiFi” 50D is an abbreviation for “wireless fidelity,” a wireless local area network (WLAN) that conforms to the Institute of Electrical and Electronics Engineers (IEEE) specification 862.11. The maximum data rate can be up to eleven megabits per second (11 Mbps). Some cellular and PCS wireless phones 16A also include WiFi capabilities.

Ultra Wide Band (UWB) 50E is a wireless technology that uses less power and provides higher data speed than WiFi or Bluetooth and has the ability to carry signals through doors and other obstacles that tend to reflect signals at more limited bandwidths and a higher power. UWB chip sets and their concomitant support software are just now becoming available. Some commercial vendors claim data rates as high as one thousand megabits per second (1,000 Mbps), although much lower rates are more realistic.

ZigBee™ 50F is a published specification set of high level communication protocols designed to use small, low power digital radios based on the IEEE 802.15.4 standard for wireless personal area networks (WPANs). The data rate for ZigBee technology is two hundred fifty kilobits per second (250 kbps) (peak information rate is one hundred twenty-eight kilobits per second (128 kbps)) compared to seven hundred twenty kilobits per second (720 kbps) for Bluetooth wireless technology.

An emerging wireless technology is Worldwide Interoperability for Microwave Access (WiMax) 50G, the IEEE 862.16 standard for broadband wireless access (BWA).

Additional short range wireless technologies are just being commercially announced that provide the similar functionality.

Wireless connections 50C, 50D, 50E, 50F, 50G between a VEMR 48 and a specific device or terminal 16, a VVB 16A1, are shown in FIG. 19.

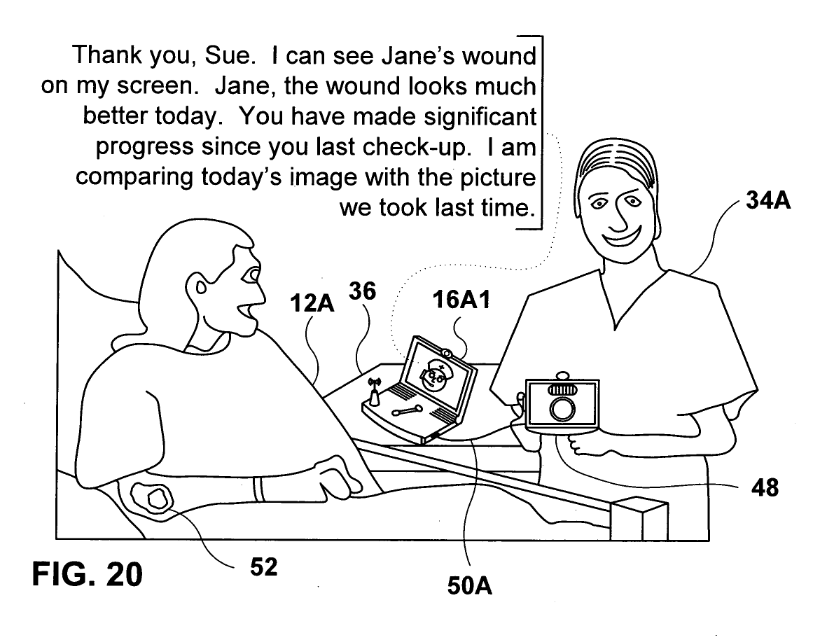

In FIG. 20 the healthcare provider 20C tells the assistant 34A that he or she can see the image 44C of the patient's 12A wound 52 as is being captured by the VEMR 48 and transmitted 32A by the VVB 16A1 to a healthcare provider's 20C diagnostic, display and control means 24A. The healthcare provide 20C also tells the patient 12A and the assistant 34A that he or she is comparing the image 44C of the wound 52 with an earlier image 44D.

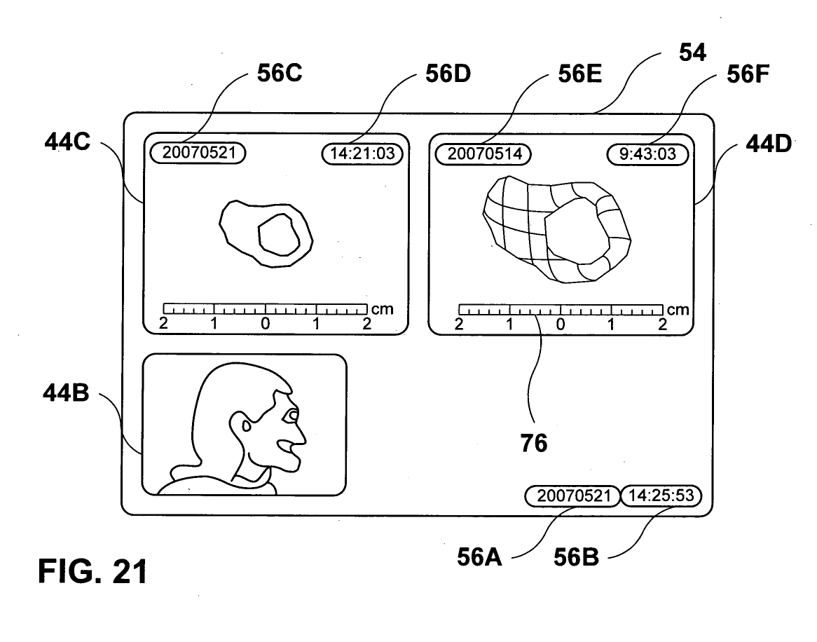

FIG. 21 shows a screen 54 on the healthcare provider's 20C diagnostic, display and control means 24A. The screen 54 shows the image 44B of the patient 12A being captured by the camera 42A on the VVB 16A1, the image 44C of the wound 52 being captured by the VEMR 48, and an earlier image 44D of the patient's 12A wound 52 that had been stored in the patient's 12A Electronic Medical Record (EMR). FIG. 21 also shows the current date 56A and time 56B, the date 56C and time 56D when the image 44C of the wound 52 was captured, and the date 56E and time 56F that the earlier image 44D was captured. Finally, FIG. 21 shows that when captured each image 44 includes a measurement scale 58 to aid in comparing images 44. The measurement scale 58 may be added to an image 44 by the VEMR 48.

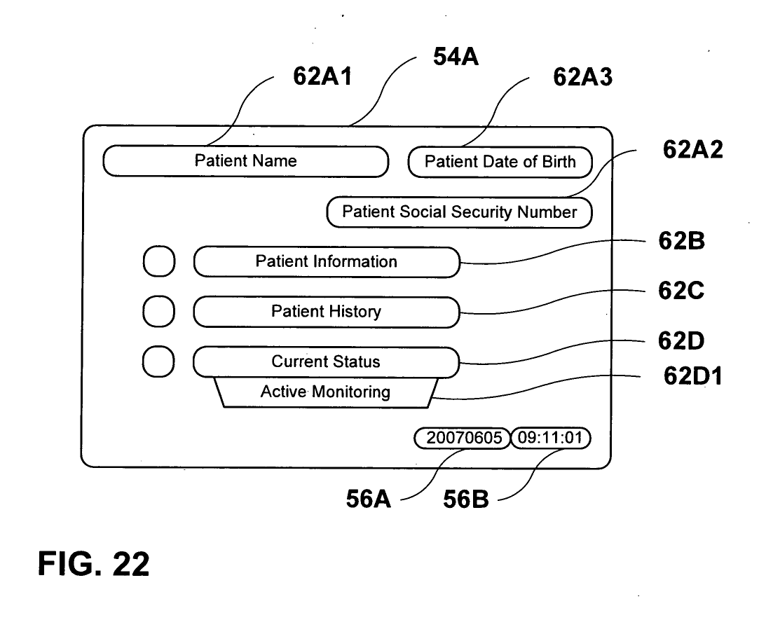

An embodiment of a first screen 60A of a patient's 12A Electronic Medical Record (EMR) 60 is shown in FIG. 22 as displayed on a screen 54A of a healthcare provider's 20C diagnostic, display and control means 24A. This first screen 60A shows the major categories 62 of the EMR 60, patient identifying information 62A such as patient name 62A1, Social Security Number 62A2 and date of birth 62A3; detailed patient information 62B; patient history 62C; and current status 62D. If a patient 12A is currently being monitored the patient's 12A current status 62D is highlighted 62D1. Clicking on one of the major category 62 names takes the healthcare provider 20C or other user to the information of that category 62.

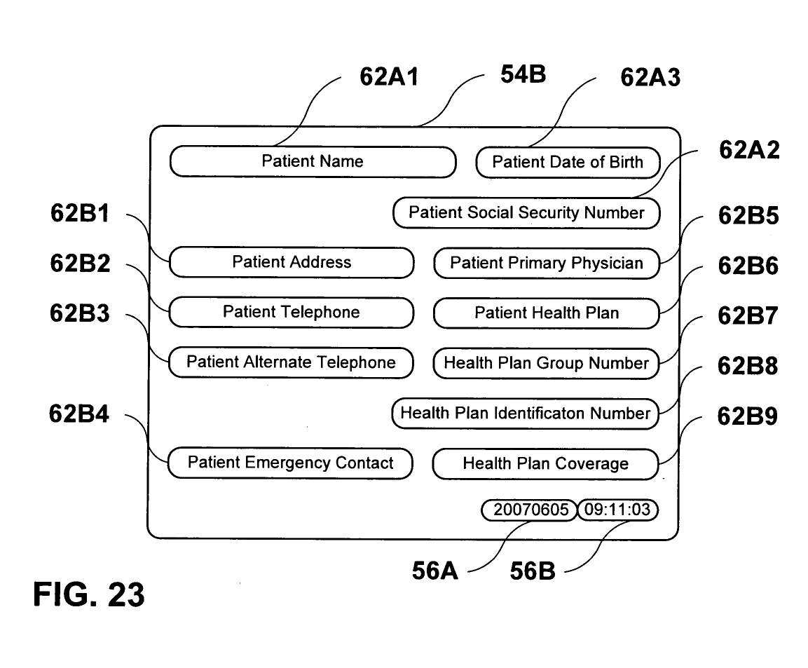

FIG. 23 shows an embodiment of a screen 60B showing detailed patient information 62B as displayed on a healthcare provider's 20C diagnostic, display and control means 24A. FIG. 23 shows patient address 62B1, patient telephone number 62B2, patient alternate telephone number 62B3, patient emergency contact 62B4, patient primary physician 62B5, patient health plan 62B6, patient health plan group number 62B7, patient health plan identification number 62B8, and a description of patient health plan coverage 62B9. FIG. 23 also shows the current date 56A and time 56B. There may be a wide variety of detailed patient information 62B beyond that shown in FIG. 23.

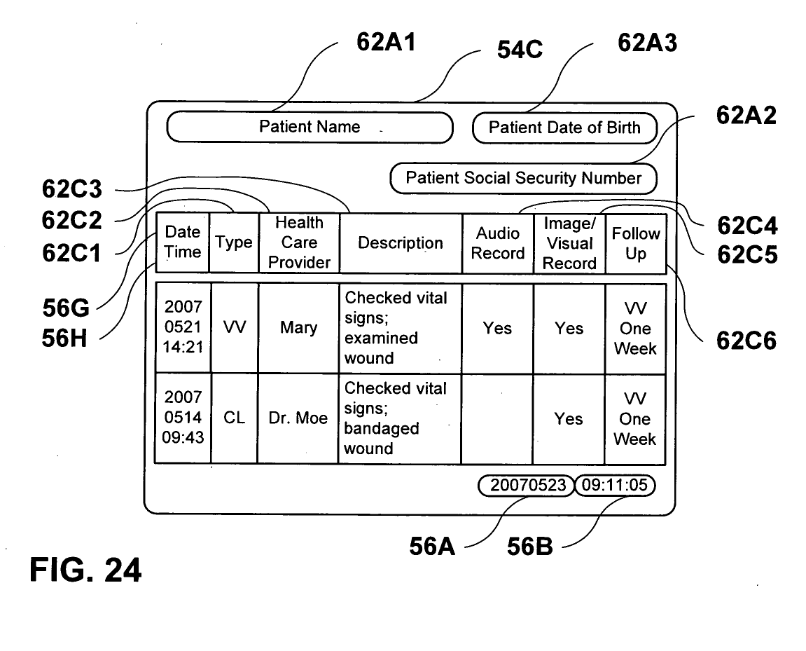

FIG. 24 shows an embodiment of a screen 60C showing patient history 62C as displayed on a healthcare provider's 20C diagnostic, display and control means 24A. This embodiment shows the date 56G and time 56H of each patient 12A medical visit, the type of visit 62C1, here “CL” means clinic and “VV” means a “Virtual Visit,” the name of the healthcare provider 62C2, a description of the medical services provided 62C3, whether an audio record of the medical visit is available 62C4, whether an image or video record of the medical visit is available 62C, and follow-up instructions 62C6. Clicking, for example, on the image/video box in each record would bring up on the screen images 44C and 44D, as shown in FIG. 21.

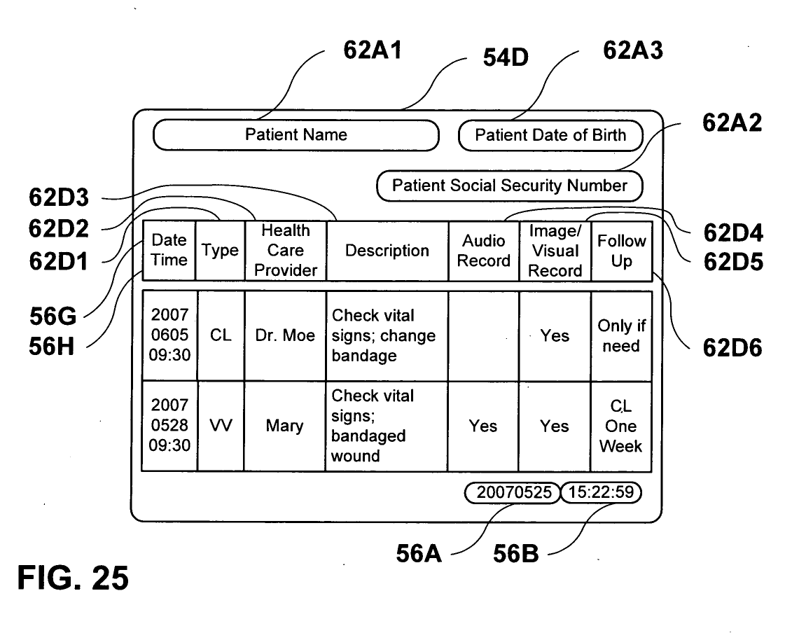

FIG. 25 shows an embodiment of a screen 60D showing the current status of the patient 12A as displayed on a healthcare provider's 20C diagnostic, display and control means 24A. This embodiment shows whether any further medical visits are planned and their schedule and character. This embodiment shows the date 561 and time 56J of a patient's 12A scheduled medical visits, the type of visit 62D2, here “CL” means clinic and “VV” means a “Virtual Visit,” the name of the Healthcare provider 62D3, a description of the medical services to be provided 62D4, whether an audio record of the medical visit is desired 62D5, whether an image or video record of the medical visit is desired 62D6, and follow-up instructions 62D7, if any.

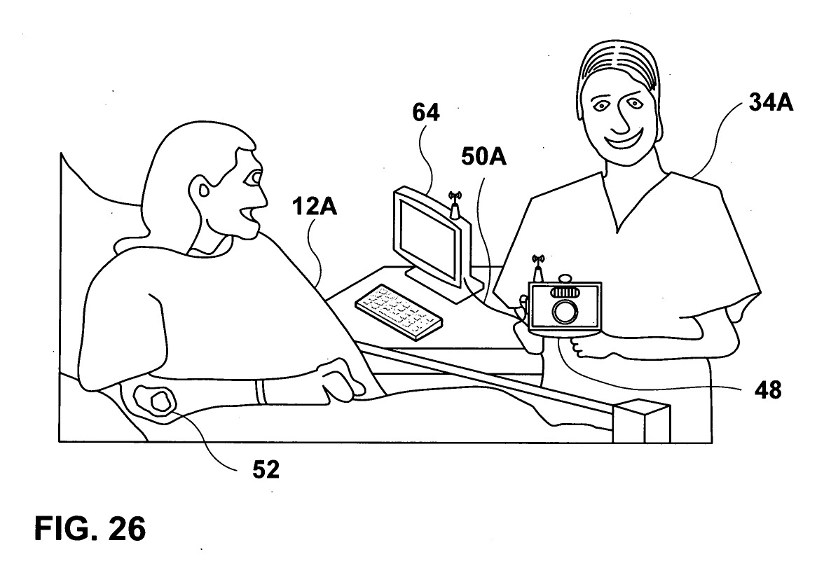

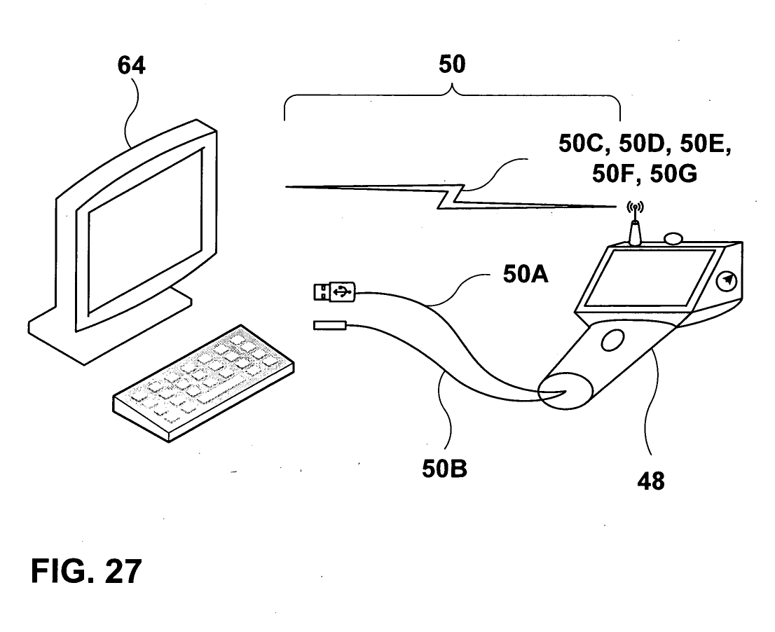

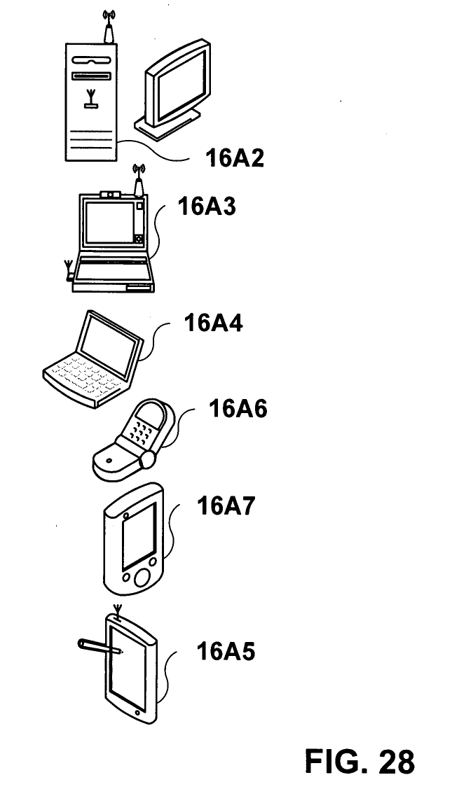

Use of the Visual Electronic Medical Record (VEMR) device 48 is not restricted to a Virtual Visit Book (VVB™) 16A1. Many medical examining rooms 14B and patient rooms in hospitals or clinics 14C have computer terminals 64 for making entries into an Electronic Medical Record (EMR) 60. A VEMR 48 may be connected to such a terminal 64 as shown in FIG. 26. In fact, a VEMR 48 may be connected to a terminal 64 via any of the methods shown in FIG. 27, wired 50A, 50B or wirelessly 50C, 50D, 50E, 50F, 50G.

Alternative embodiments of the Virtual Visit Book™ (VVB) 16A1 are any device with at a minimum a transceiver, a display, a camera and capable of an audio connection, including, but not limited to, a personal computer (PC) 16A2, laptop PC 16A3, ultra-mobile PC (UMPC) 16A4, tablet PC 16A5, cellular or Personal Communications Service (PCS) phone 16A6, or Personal Digital Assistant (PDA) or “SmartPhone” 16A7.

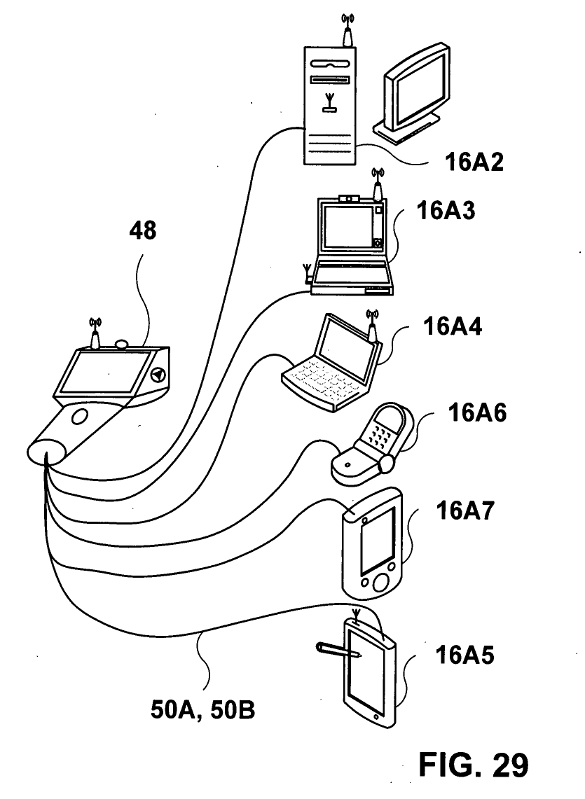

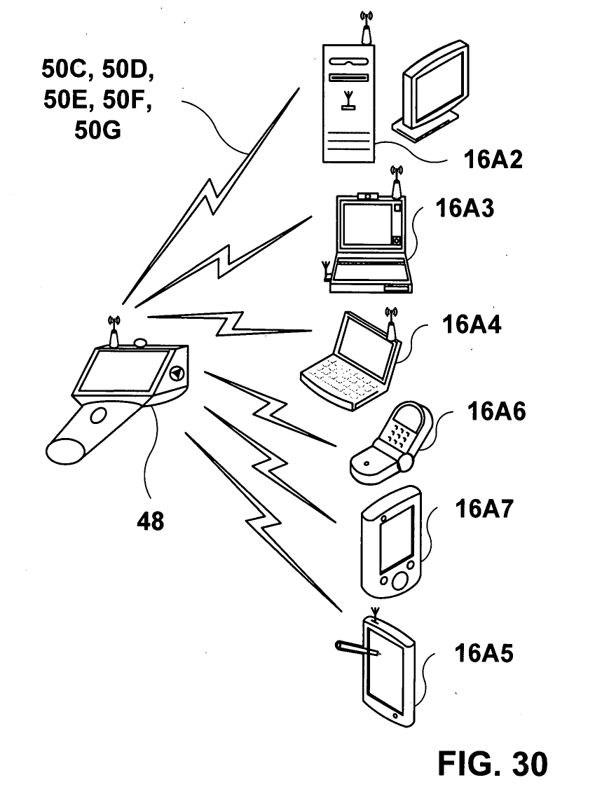

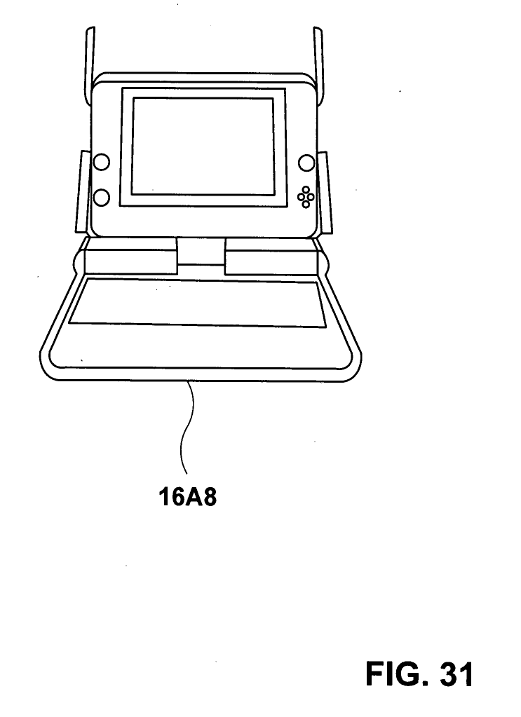

A Visual Electronic Medical Record (VEMR™) device 48 may be connected to any of these alternative embodiments of the Virtual Visit Book™ (VVB) 16A2 through 16A7 either wired 50A, 50B as shown in FIG. 29 or wirelessly 50C through 50G as shown in FIG. 30.

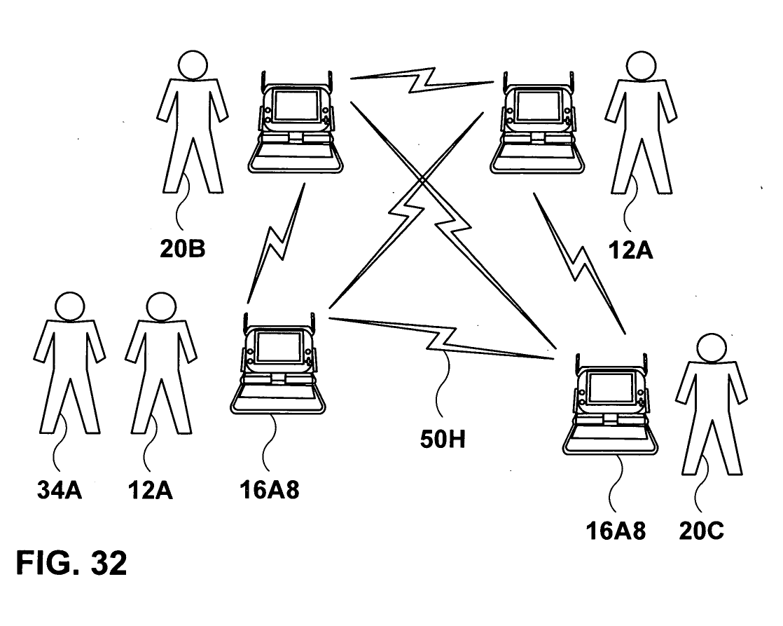

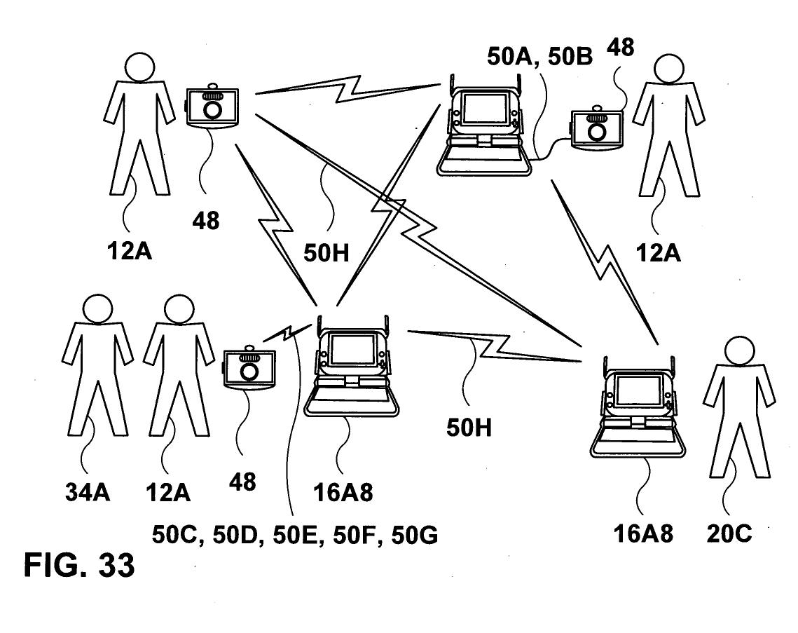

A particular alternative embodiment of the Virtual Visit Book™ (VVB) 16A1 is the One Laptop Per Child's (OLPC) XO laptop 16A8 (more commonly although not completely accurately known as the “$100 laptop”), shown in FIG. 31. The objective of the OLPC project is to bring inexpensive computing resources to children in the developing world. One of the XO's capabilities is wireless mesh networking 50H, which will enable interconnection of numbers of XOs 16A8 as well as other Virtual Visit Books 16A1 through 16A7 having similar wireless mesh networking 50H capabilities. Wireless mesh networking 50H not only enables remote diagnosis, triage and health status monitoring of patients 12A, it also enables consultation amongst multiple healthcare providers 20B, 20C, as shown in FIG. 32.

A Visual Electronic Medical Record (VEMR™) device 48 may be connected to this alternative embodiment of the Virtual Visit Book (VVB) 16A8 as shown in FIG. 33, either wired 50A, 50B or wirelessly 50C through 50G. If the VEMR includes wireless mesh networking 50H capabilities, it would appear as just another node on the wireless network and its information would be available to all other nodes.

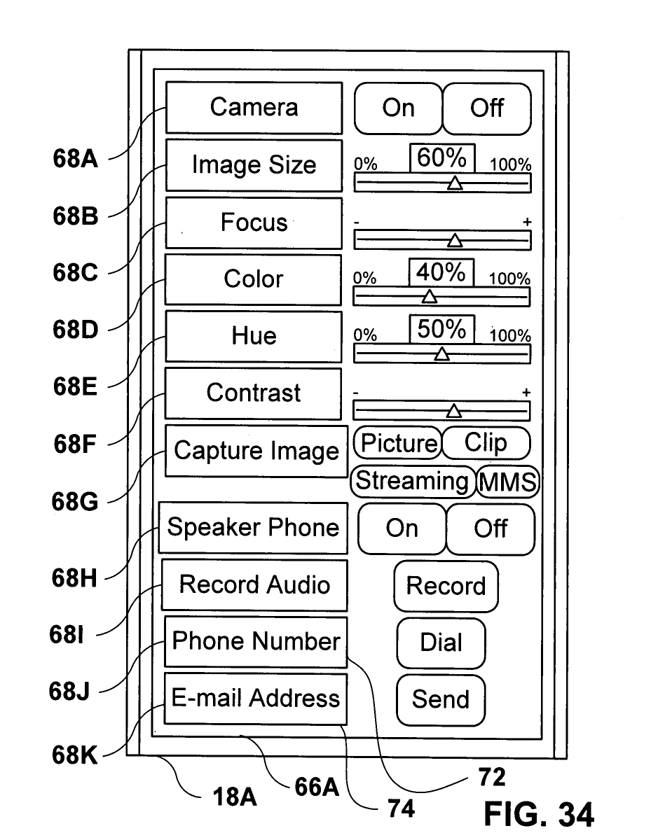

A first preferred embodiment of a functional block diagram 66A of the embedded software 18A that enables remote function control of a remote diagnostic means 16A, a Virtual Visit Book™ (VVB) 16A1, is shown in FIG. 34. The functions 68 of a remote diagnostic means 16A to be controlled include, but are not limited to:
Turn the camera 42A on or off 68A;
“Zoom” the camera image 44B size 68B;
Adjust the camera 42A focus 68C;
Adjust the camera 42A color 68D;
Adjust the camera 42A hue 68E;
Adjust the camera 42A contrast 68F;
Capture 68G an image 44B;
Turn 68H the speaker phone 40 on or off;
Record 68I the Virtual Visit;
Enter 68J a telephone number 72 to which to send an image 44B or audio recording 70; and
Enter 68K an electronic mail (e-mail) address 74 to which to send the captured image 44B.

The image 44B displayed on the screen 54 on the healthcare provider's 20C diagnostic, display and control means is being captured by the camera 42A on the VVB 16A1. The image 44B is a continuous or semi-continuous viewing of what the camera 42A “sees.” That image 44B can be acquired and manipulated as a picture 44B1, streaming video 44B2, a video clip 44B3 or a multi-media message (MMS) 44B4. The streaming video 44B2, a video clip 44B3 or a multi-media message (MMS) 44B4 may include audio 70A.

The telephone number 72 or the electronic mail address 74 may be to send an image 44B or audio recording 70 to a specialist 20B or even the patient 12A himself or herself. Any image 44B or audio recording 70 is automatically captured in the patient's 12A Electronic Medical Record (EMR) 60.

The date 56C and time 56D of the image 44B are captured automatically by the software 26A.

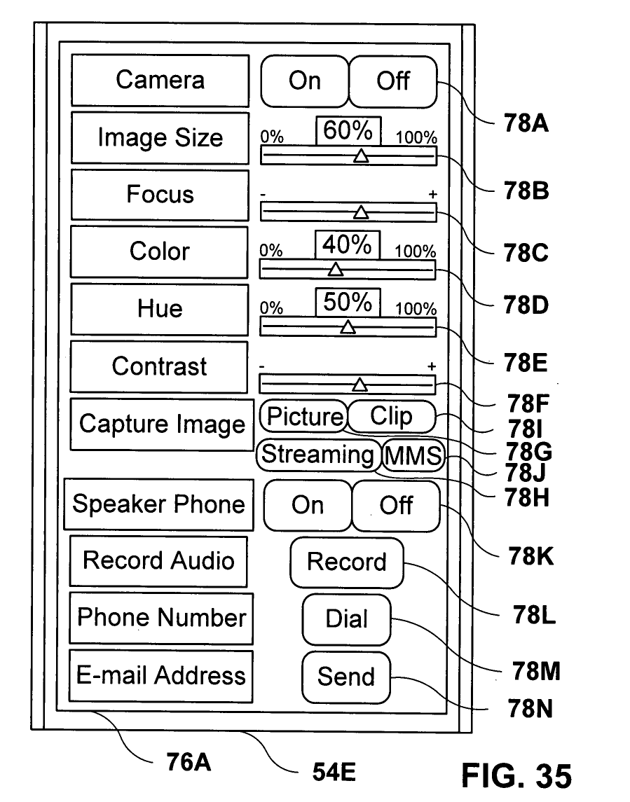

A first preferred embodiment of a block diagram 76A for the diagnostic, display and control software application 26A deployed on a diagnostic, display and control means 24A used by a healthcare provider 20C to control a remote diagnostic means 16A, a Virtual Visit Book™ (VVB) 16A1, is shown in FIG. 35. The remote functional control means 78 for a remote diagnostic means 16A include, but are not limited to:
An “off” button and an “on” button 78A for controlling 68A the camera 42A;
A slider 78B that “zooms” the camera image 44B size 68B from zero percent (0%) to one hundred percent (100%);
A slider 78C that adjusts the camera focus 68C from minus (−) to plus
A slider 78D that adjusts the color 68D from zero percent (0%) to one hundred percent (100%);
A slider 78E that adjusts the hue 68E from zero percent (0%) to one hundred percent (100%);
A slider 78F that adjusts the contrast 68F from minus (−) to plus (+);
A button 78G for capturing 68G the image 44B as a picture 44B1;
A button 78H for capturing 68G the image 44B as streaming video 44B2;
A button 78I for capturing 68G the image 44B as a video clip 44B3;
A button 78J for capturing 68G the image 44B as a multi-media message (MMS) 44B4;
An “off” button and an “on” button 78K for controlling 68H the speaker phone 40;
A button 78L for recording 68I the Virtual Visit;
A button 78M for dialing 68J the phone number 72 to which the image 44B or audio recording 70 is to be sent; and
A button 78N for sending 68K the image 44B or audio recording 70 to an e-mail address 74.

The functional control means 78 will appear on the screen 54 of the diagnostic, display and control means 24A when a healthcare provider 20C elects to take functional control of a remote diagnostic means 16A as shown in FIG. 35.

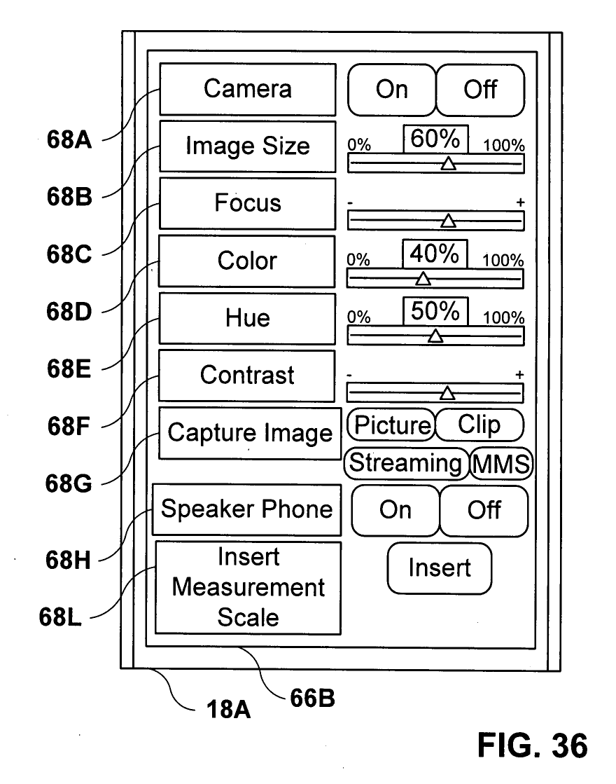

A first preferred embodiment of a functional block diagram 66B of the embedded software 18A that enables remote function control of a Visual Electronic Medical Record (VEMR™) device 48, is shown in FIG. 36. The functions 68 of the VEMR to be controlled include, but are not limited to:
Turn the camera 42A on or off 68A;
“Zoom” the camera image 44C size 68B;
Adjust the camera 42A focus 68C;
Adjust the camera 42A color 68D;
Adjust the camera 42A hue 68E;
Adjust the camera 42A contrast 68F;
Turn illumination 48C on or off 68J;
Adjust the illumination 68K;
Capture 68G an image 44C;
Turn 68H the microphone 40B on or off;
Record 68I the Virtual Visit; and
Insert 68L the measurement scale 58 into the image 44C.

The image 44C displayed on the screen 54 on the healthcare provider's 20C diagnostic, display and control means is being captured by the camera 42A in the VEMR 48. The image 44B is a continuous or semi-continuous viewing of what the camera 42A “sees.” That image 44C can be acquired and manipulated as a picture 44C1, streaming video 44C2, a video clip 44C3 or a multi-media message (MMS) 44C4. The streaming video 44C2, a video clip 44C3 or a multi-media message (MMS) 44C4 may include audio 70A. Any image 44C or audio recording 70 is automatically captured in the patient's 12A Electronic Medical Record (EMR) 60.

The date 56C and time 56D of the image 44C are captured automatically by the software 26A.

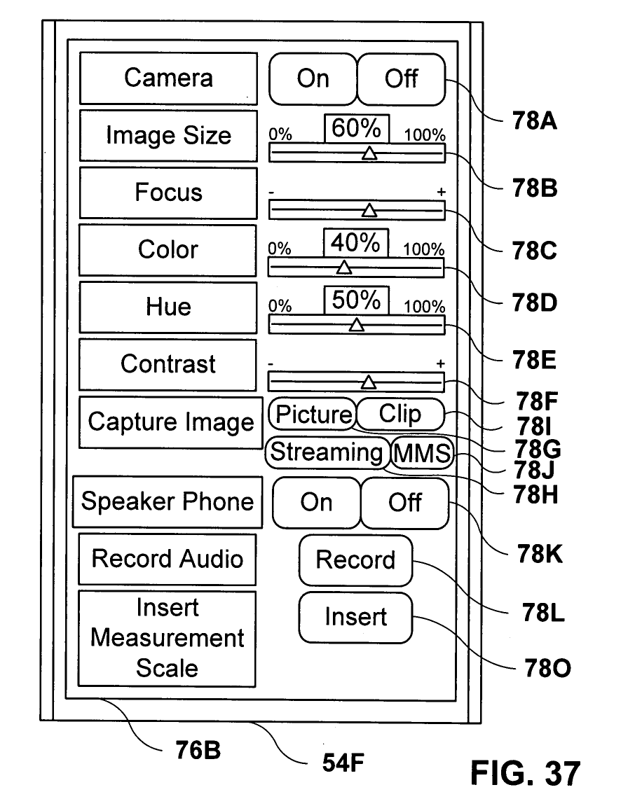

A first preferred embodiment of a block diagram 76B for the diagnostic, display and control software application 26A deployed on a diagnostic, display and control means 24A used by a healthcare provider 20C to control a Visual Electronic Medical Record (VEMR™) device 48, is shown in FIG. 37. The remote functional control means 78 for a VEMR include, but are not limited to:
An “off” button and an “on” button 78A for controlling 68A the camera 42B;
A slider 78B that “zooms” the camera image 44C size 68B from zero percent (0%) to one hundred percent (100%);
A slider 78C that adjusts the camera focus 68C from minus (−) to plus
A slider 78D that adjusts the color 68D from zero percent (0%) to one hundred percent (100%);
A slider 78E that adjusts the hue 68E from zero percent (0%) to one hundred percent (100%);
A slider 78F that adjusts the contrast 68F from minus (−) to plus (+);
An “off” button and an “on” button 78M for controlling 68F illumination 48C;
A slider 78N that adjusts the illumination 48C from zero percent (0%) to one hundred percent (100%);
A button 78G for capturing 68G the image 44C as a picture 44C1;
A button 78H for capturing 68G the image 44C as streaming video 44C2;
A button 78I for capturing 68G the image 44C as a video clip 44C3;
A button 78J for capturing 68G the image 44C as a multi-media message (MMS) 44C4;
An “off” button and an “on” button 78K for controlling 68H the microphone 40B;
A button 78L for recording 68I the Virtual Visit; and
A button 78O for inserting 58L a measurement scale 58 into an image 44C.

The functional control means 78 will appear on the screen 54 of the diagnostic, display and control means 24A when a healthcare provider 20C elects to take functional control of VEMR as shown in FIG. 37.

The date 56C and time 56D of the image 44C are captured automatically by the software 26A.

In this Specification and in the Claims that follow, the term “partially control” refers to a joint or cooperative sharing of the control of the features of a remote diagnostic means 16A, specifically including a Virtual Visit Book™ (VVB), and/or a Visual Electronic Medical Record (VEMR) device 48 by both the user and a another person, such as an operator 20A at a call center 28. The operator 20A may control some or all of the features of the remote diagnostic means 16A or VEMR 48.

### II. Data Devices

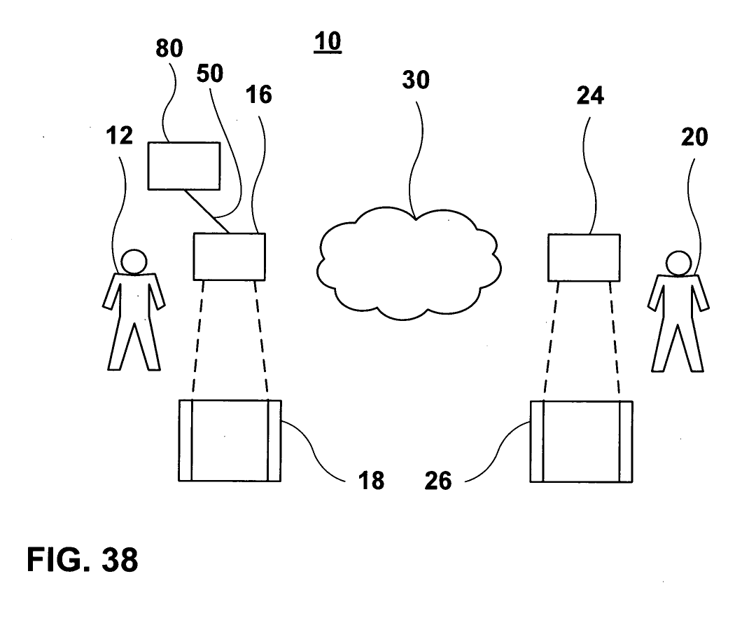

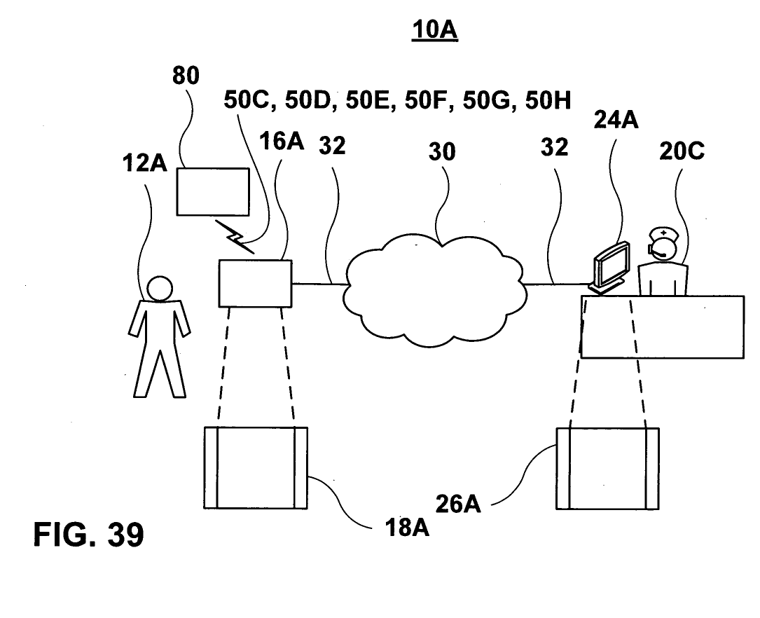

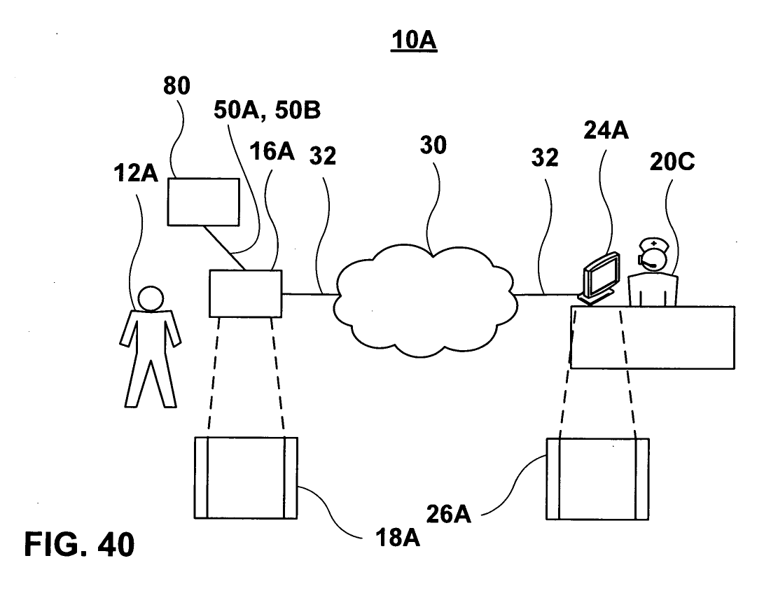

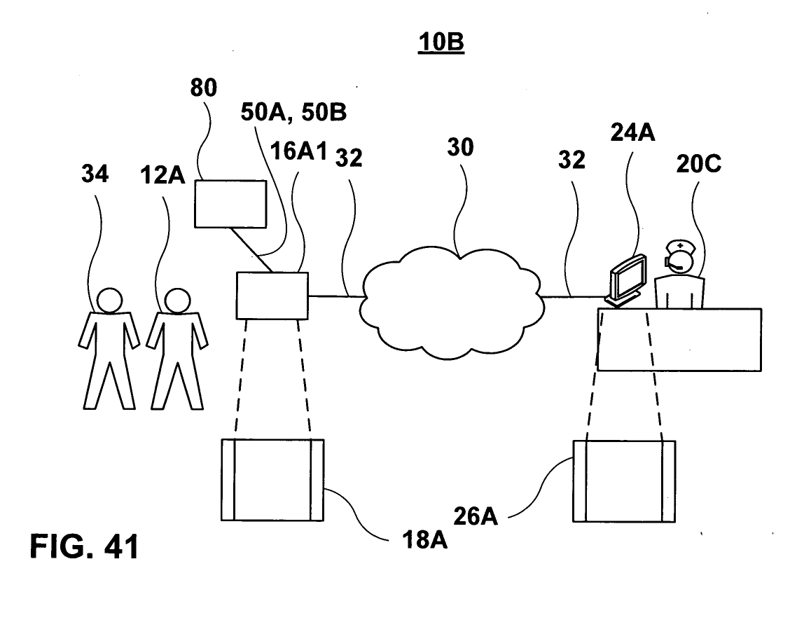

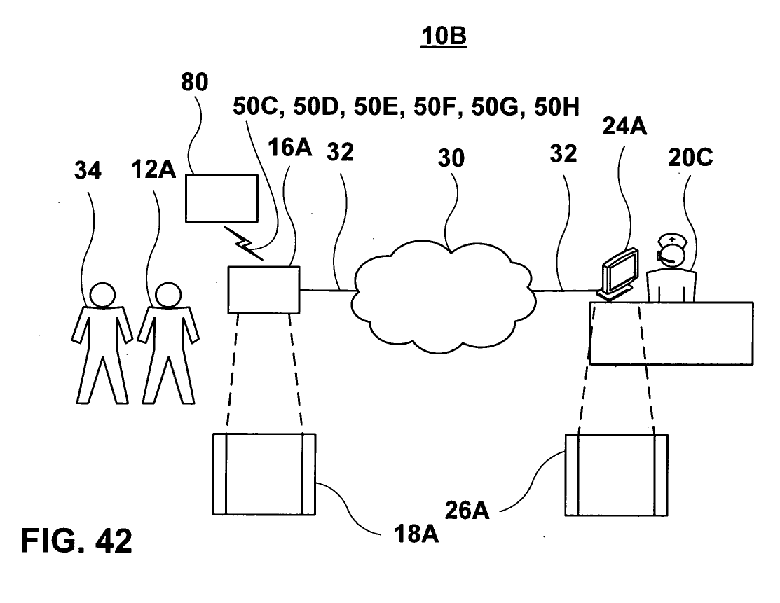

Many different data devices 80 can enhance the effectiveness of the Personal Healthcare Assistant 10. These data devices 80 are connected 50 to a device 16 via numbers of technologies, both wired and wireless, as shown in FIG. 38. Wireless connections 50C, 50D, 50E, 50F, 50G between the data devices 80 and a remote diagnostic means 16A are shown in FIG. 39. Wired connections 50A, 50B between data devices 80 and a remote diagnostic means 16A are shown in FIG. 40. Wired connections 50A, 50B between data devices 80 and a Virtual Visit Book™ (VVB) 16A1 are shown in FIG. 41. Wireless connections 50C, 50D, 50E, 50F, 50G between the data devices 80 and a Virtual Visit Book™ (VVB) 16A1 are shown in FIG. 42.

For each of the data devices 80 to connect 50 to a device 16, there must be a connection interface device 82 that accepts the data from the data device 80, and configures it for the connection 50 to the device 16.

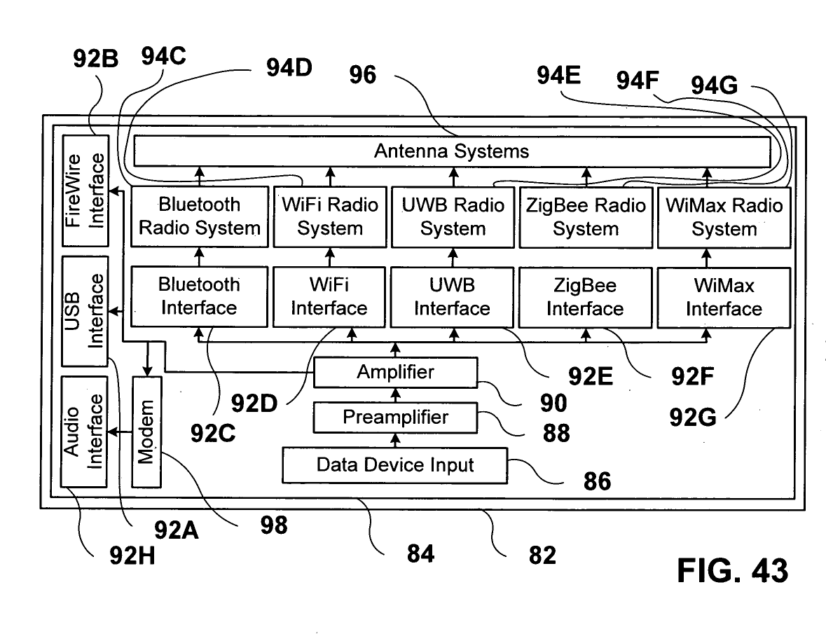

FIG. 43 shows a functional block diagram 84 for the connection interface device 98. Data device input 86 is fed to a preamplifier 78 and then an amplifier 90. Thereafter the amplified data device input 86 is fed into the appropriate interface 92 for the connection 50 to be used; the USB interface 92A for USB 50A, and the FireWire interface 92B for FireWire 50B, the Bluetooth interface 92C for Bluetooth 50C, the WiFi interface 92D for WiFi 50D, the UWB interface 92E for UWB 50E, the ZigBee interface 92F for ZigBee 50F, and the WiMax interface 92G for WiMax 50G. The output of the USB interface 92A is the USB connection 50A; the output of the FireWire interface 92B is the FireWire connection 50B. The output of the Bluetooth interface 92C is fed into the Bluetooth radio system 94C and then to the antenna system 96. Similarly, the output of the WiFi interface 92B is fed to into the WiFi radio system 94D and then to the antenna system 96. The output of the UWB interface 92E is fed into the UWB radio system 94E and then to the antenna system 96. The output of the ZigBee interface 92F is fed into the ZigBee radio system 94F and then to the antenna system 96. The output of the WiMax interface 92G is fed into the WiMax radio system 94G and then to the antenna system

The earliest data transmissions used modems connected to phone lines. Digital data was converted to audio signals that could reliably be transmitted over phone lines and converted back to digital data at the other end of the transmission. A similar technique may be employed here. Digital data 86 from the data devices 80 is sent to a modem 98 and then fed into an audio interface 92H. See FIG. 43.

In one embodiment, the connection interface device 98 is built into the data devices 80. In one embodiment, one or more connection 50 technologies is built into each data device 80.

### Thermometer

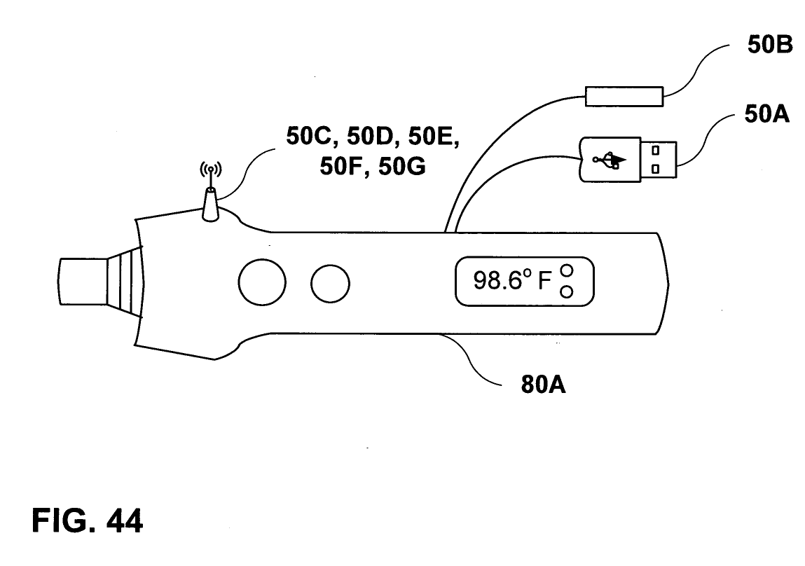

The first data device 80 is a thermometer 80A. Many different contact-less digital thermometers 80A are commercially available from Bebesounds®, Braun®, EJK®, Lumiscope®, Mabis Healthcare®, Samsung® and others. A preferred embodiment of a thermometer 80A to be deployed in the Personal Healthcare Assistant 10A is shown in FIG. 44, and includes a connection 50 to a device or terminal 16. The embodiment shown in FIG. 44 includes one or more wired connections, USB 50A and FireWire 50B, or one or more wireless connections, Bluetooth 50C, WiFi 50D, UWB 50E, ZigBee 50F and WiMax 50G.

For a diagnostic device 16A to receive the temperature reading 68P from the thermometer 80A, it must have embedded software 18A that recognizes that a digital temperature reading is being sent to the diagnostic device 16A. One embodiment of the embedded software 18A allows the temperature 68P to be displayed on the screen 38 of a diagnostic device 16A. Having received the temperature reading 68P from the thermometer 80A, there must be additional software 18A to forward the temperature reading to the diagnostic, display and control software application 26A deployed on the diagnostic, display and control means 24A used by a healthcare provider 20C.

Similarly, the diagnostic, display and control software application 26A deployed on a diagnostic, display and control means 24A used by a healthcare provider 20C has to have the ability to display the temperature reading 68P received from the diagnostic device 16A. The healthcare provider 20C must have the ability to take or retake the temperature reading 86A, as well as to determine whether to display the temperature reading 68P on the diagnostic device 16A. Finally, the healthcare provider 20C can save the temperature reading 68P to the patient's Electronic Medical Record (EMR) 60.

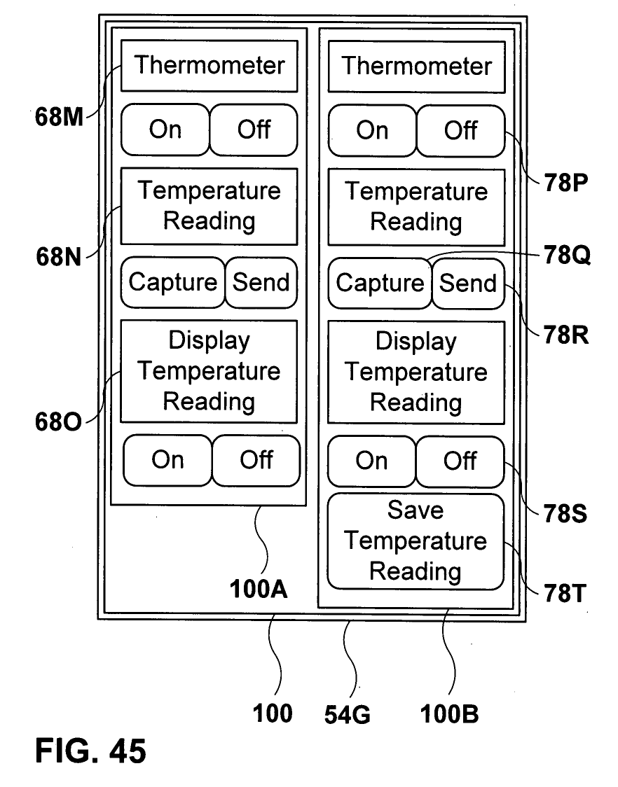

FIG. 45 shows a first embodiment of a functional block diagram 100 of embedded software 18A that enables remote functional control of the data devices 80 for the diagnostic means 16A as well as a software application 26A that enables remote functional control of the data devices 80 connected to the diagnostic means 16A that appears on a seventh screen 58G of the diagnostic, display and control means 24A. The functions shown in 100A are deployed as a component of 18A, the software embedded in a diagnostic means 16A; those in 100B as a component of the diagnostic, display and control software application 26A.

The functions 68 embedded in a diagnostic device 16A for controlling the data device 80 the thermometer 80A include, but are not limited to:
Turn the thermometer 80A on or off 68O;
Capture and send the temperature reading 68P; and
Turn the temperature reading display 68Q of the diagnostic means 16A on or off.

The functional control means 78 in the diagnostic, display and control software application 26A for remotely controlling the thermometer 80A via a diagnostic means 16A include, but are not limited to:
An “off” button and an “on” button 78R for controlling 68M the thermometer 80A;
A button 78S for capturing or recapturing the temperature reading 68P;
A button 78T for sending the temperature reading 68P to a healthcare provider 20C; and
An “off” button and an “on” button 78U for controlling the display 68O of the temperature reading 68P on a diagnostic means 16A.

An additional functional control means 78V allows the healthcare provider 20C to save the temperature reading 68P to the patient's 12A Electronic Medical Record (EMR) 60. The diagnostic, display and control software application 26A automatically tags the temperature reading 68P with the date 56A and time 56B.

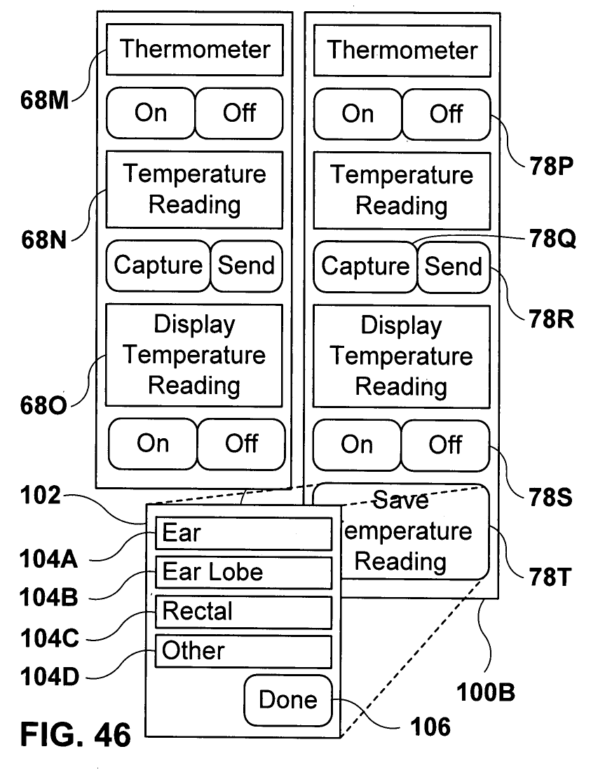

When button 78V is pushed, pop-up window 102 appears on the diagnostic, display and control means 24A allowing the healthcare provider 20C to note where on the patient's 12A body the temperature reading 68P was taken 104, as shown in FIG. 46. If the location 104 is not listed in pop-up window 100, the healthcare provider 20C enters the location in the “other” box 104D. Selecting one of 104A through 104C automatically closes pop-up window 102. If information is entered into 104D, the healthcare provider 20C clicks the “done” button 106 to close the pop-up window 102.

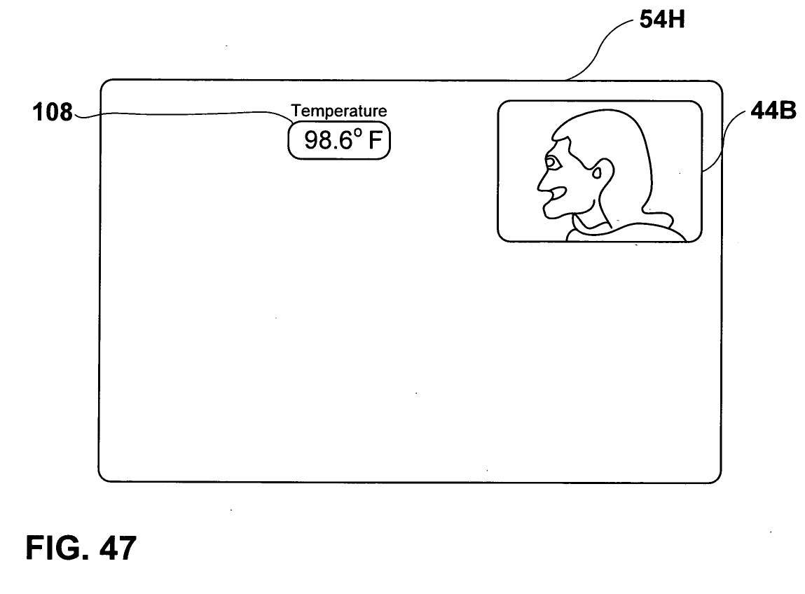

The temperature reading 68P appears in window 108 on the healthcare provider's 20C diagnostic, display and control means 24A as shown in FIG. 47.

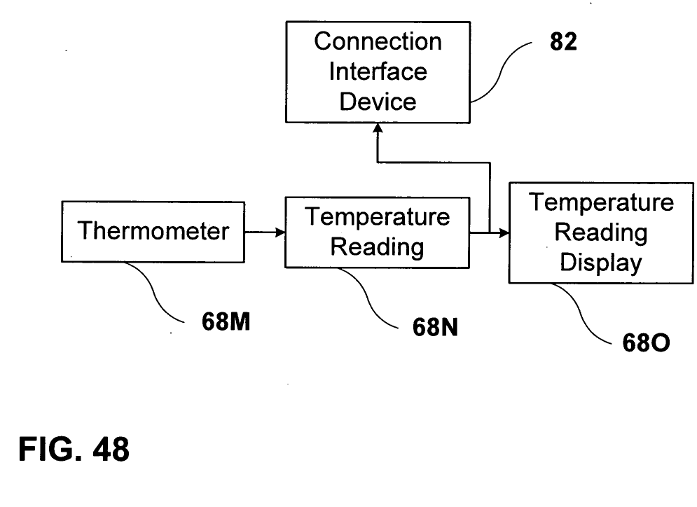

FIG. 48 shows the temperature reading 68P fed to the connection interface device 82 for connection 50 to the device or terminal 16, including via the audio interface 92H.

### Stethoscope or High Fidelity Microphone

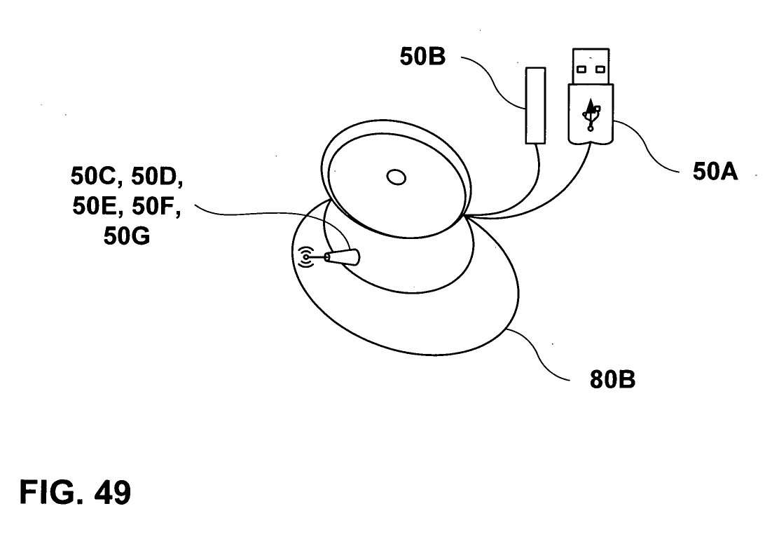

A second data device 80 is an acoustic sensor, such as a stethoscope or high fidelity microphone 80B. A stethoscope or high fidelity microphone 80B is used to listen to the heart and lungs 86B of a patient or potential patient 12A as well as to capture pulse rate 86C. Numbers of stethoscopes 80B are commercially available from AllHeart®, American Diagnostic Corporation (ADC)®, Doctors Research Group (DRG)®, Heine®, Prestige Medical®, 3M Littmann®, UltraScopes®, W.A. Baum®, WelchAllyn® and others. High fidelity microphones are commercially available from AKG®, Audio-Technica®, Beyerdynamics®, Sennheiser®, Shure®, Sony® and others. A preferred embodiment of a stethoscope or high fidelity microphone 80B to be deployed in the Personal Healthcare Assistant 10A is shown in FIG. 49, and includes a connection 50 to a device 16. The embodiment shown in FIG. 25 includes wired connections, USB 50A and FireWire 50B, and one or more wireless connections, Bluetooth 50C, WiFi 50D, UWB 50E, ZigBee 50F and WiMax 50G.

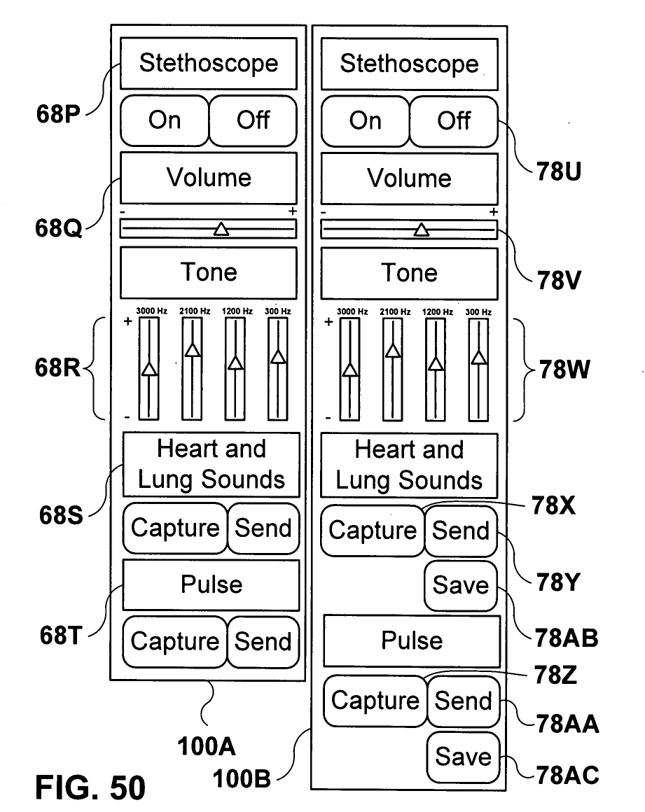

FIG. 50 shows a second embodiment of a functional block diagram 100 of embedded software 18A that enables remote functional control of the data devices 80 for the diagnostic means 16A as well as a software application 26A that enables remote functional control of the data devices 80 connected to the diagnostic means 16A. The functions shown in 100A are deployed as a component of 18A, the software embedded in a diagnostic means 16A; those in 100B as a component of the diagnostic, display and control software application 26A.

The pulse rate 68V is automatically captured by either the embedded software that enables remote function control 18A or the diagnostic, display and control software application 26A, basically by listening to the heart beats 68U and measuring them against the elapsed time 56K.

The functions 68 embedded in a diagnostic device 16A for controlling the data device 80 the stethoscope or high fidelity microphone 80B include, but are not limited to:
Turn the stethoscope or high fidelity microphone 80B on or off 68R;
Adjust the volume 68S of the stethoscope or high fidelity microphone 80B;
Adjust the tone 68T of the stethoscope or high fidelity microphone 80B using an equalizer;
Capture heart and lungs sounds 68U; and
Capture the pulse 68V.

The functional control means 78 in the diagnostic, display and control software application 26A for remotely controlling the stethoscope or high fidelity microphone 80B via a diagnostic device 16A include, but are not limited to:
An “off” button and an “on” button 78W for controlling 68R the stethoscope or high fidelity microphone 80B;
A slider 78X that adjusts the volume 68S of the stethoscope or high fidelity microphone 80B from minus (−) to plus (+);
Multiple sliders 78Y that adjust the tone 68T of the stethoscope or high fidelity microphone 80B from minus (−) to plus (+);
A button 78Z for capturing or recapturing heart and lung sounds 68U;
A button 78AA for sending the heart and lung sounds 68U to a healthcare provider 20C;
A button 78AB for capturing or recapturing the pulse reading 68V; and
A button 78AC for sending the pulse reading 68V to a healthcare provider 20C.

An additional functional control means 78AD allows the healthcare provider 20C to save the heart and lung sounds 68U to the patient's 12A Electronic Medical Record (EMR) 60. The software application 26A automatically tags the heart and lung sounds 68U with the date 56A and current time 56B.

An additional functional control means 78AE allows the healthcare provider 20C to save the pulse reading 68V to the patient's 12A Electronic Medical Record (EMR) 60. The software application 26A automatically tags the pulse reading 68V with the date 56A and current time 56B.

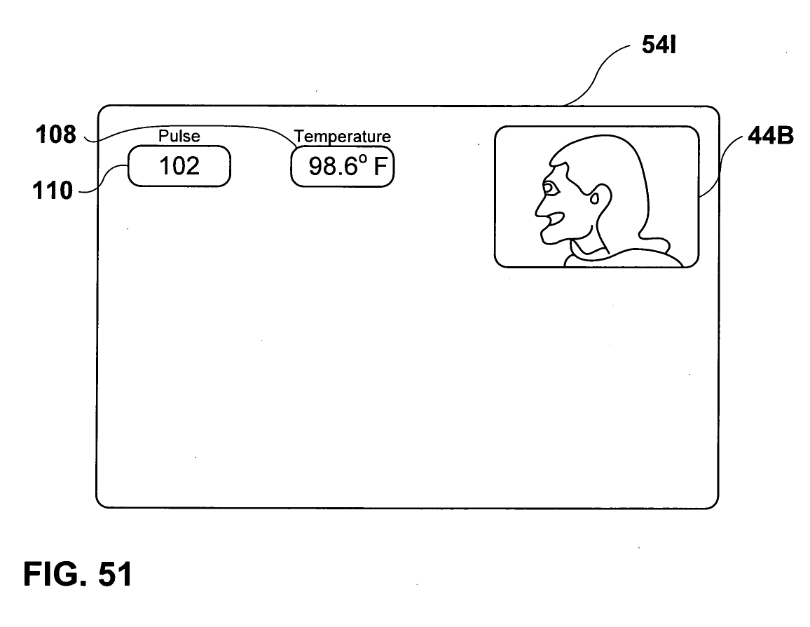

The pulse reading 68V appears in window 110 on the healthcare provider's 20C diagnostic, display and control means 24A as shown in FIG. 51.

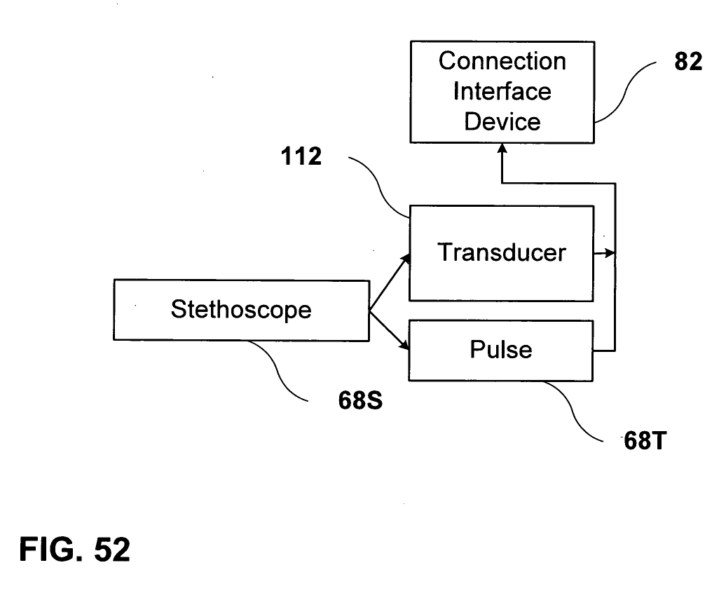

A stethoscope is basically a cavity resonator that amplifies sound; there are no electronic components. Microphone components, a transducer 112, must be added to a stethoscope to convert sound waves to electrical signals. The stethoscope or high fidelity microphone 80B must have an interface that captures the sound signals and makes those signals available to the connection 50 to the device or terminal 16. This is accomplished via the connection interface device 82 shown in FIG. 52. If the sound is to be passed directly to the audio interface 92H, no transducer 112 is required.

### Weight Scale

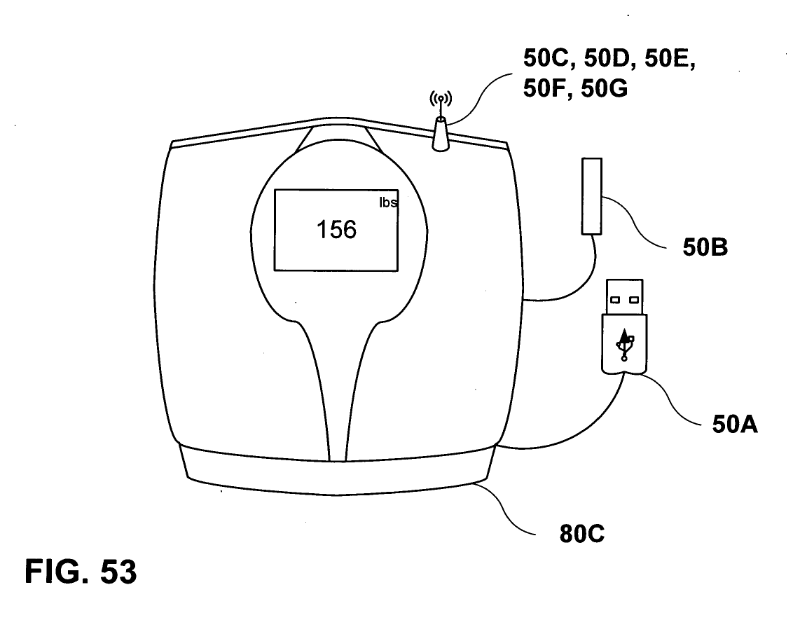

The third data device 80 is a weight measurement device, such as a scale 80C. Numbers of weight scales 80C are commercially available from Braun®, Health-O-Meter®, Homedics®, LifeSource®, MedWeigh®, Rowenta®, Soehnle®, Tanita® and others. A preferred embodiment of a weight scale 80C to be deployed in the Personal Healthcare Assistant 10A is shown in FIG. 53, and includes a connection 50 to a device or terminal 16. The embodiment shown in FIG. 53 includes one or more wired connections, USB 50A and FireWire 50B, and one or more wireless connections, Bluetooth 50C, WiFi 50D, UWB 50E, ZigBee 50F and WiMax 50G.

For a diagnostic device 16A to receive the weight reading 68X from the scale 80C, it must have embedded software 18A that recognizes that a digital weight reading is being sent to the diagnostic device 16A. One embodiment of the embedded software 18A allows the weight to be displayed on the screen 38 of the diagnostic device 16A. Having received the weight reading 68X from the scale 80C, there must be additional software 18A to forward the weight reading to the diagnostic, display and control means 24A used by a healthcare provider 20C.

Similarly, the diagnostic, display and control software application 26A deployed on a diagnostic, display and control means 24A used by a healthcare provider 20C has to have the ability to display the weight reading 68X received from the diagnostic device 16A. The healthcare provider 20C must have the ability to take or retake the weight reading 68X, as well as to determine whether to display the weight reading 68X on the diagnostic device 16A.

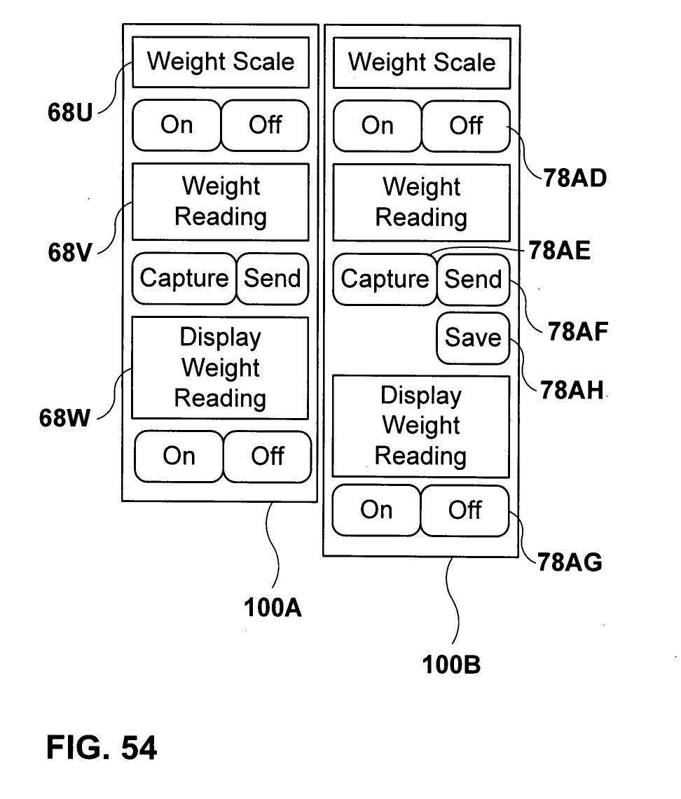

FIG. 54 shows a third embodiment of a functional block diagram 100 of embedded software 18A that enables remote functional control of the data devices 80 for the diagnostic means 16A as well as a software application 26A that enables remote functional control of the data devices 80 connected to the diagnostic means 16A. The functions shown in 100A are deployed as a component of 18A, the software embedded in a diagnostic means 16A; those in 100B as a component of the diagnostic, display and control software application 26A.

The functions 68 embedded in a diagnostic device 16A for controlling the data device 80 the weight scale 80C include, but are not limited to:
Turn the scale 80C on or off 68W;
Capture and send the weight reading 68X; and
Turn the weight reading display 68Y on or off.

The functional control means 78 in the diagnostic, display and control software application 26A for remotely controlling the scale 80C via a diagnostic device 16A include, but are not limited to:
An “off” button and an “on” button 78AF for controlling 68W the scale 80C;
A button 78AG for capturing or recapturing the weight reading 68X;
A button 78AH for sending the weight reading 68X to a Healthcare provider 20C; and
An “off” button and an “on” button 78AI for controlling the display 68Y of the weight reading 68X.

An additional functional control means 78AJ allows the healthcare provider 20C to save the weight reading 68X to the patient's 12A Electronic Medical Record (EMR) 60. The software application 26A automatically tags the weight reading 68X with the date 56A and current time 56B.

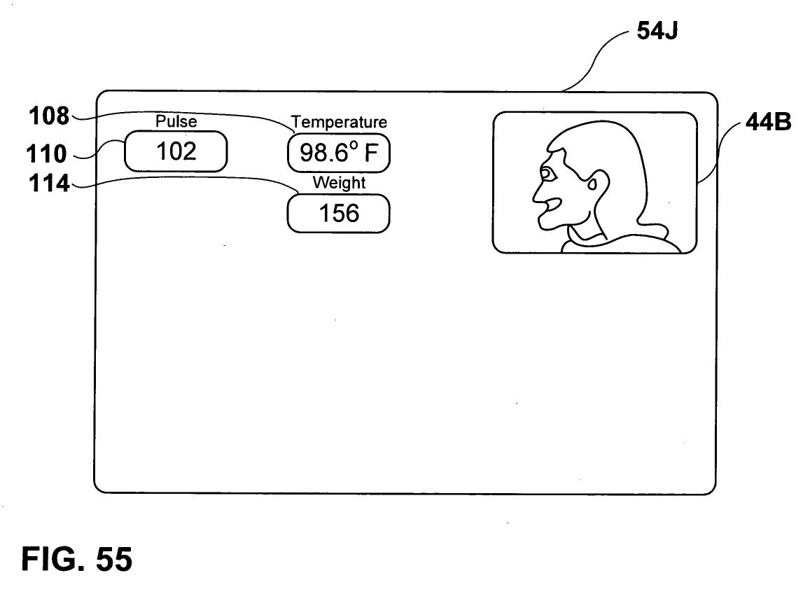

The weight reading 68X appears in window 114 on the healthcare provider's 20C diagnostic, display and control means 24A as shown in FIG. 55.

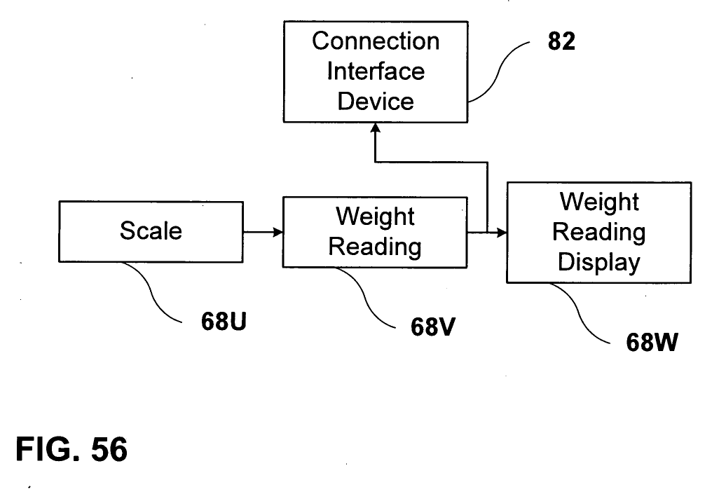

FIG. 56 shows the weight reading 68X fed to the connection interface device 82 for connection 50 to the device or terminal 16 including, via the audio interface 92G.

### Blood Pressure Cuff

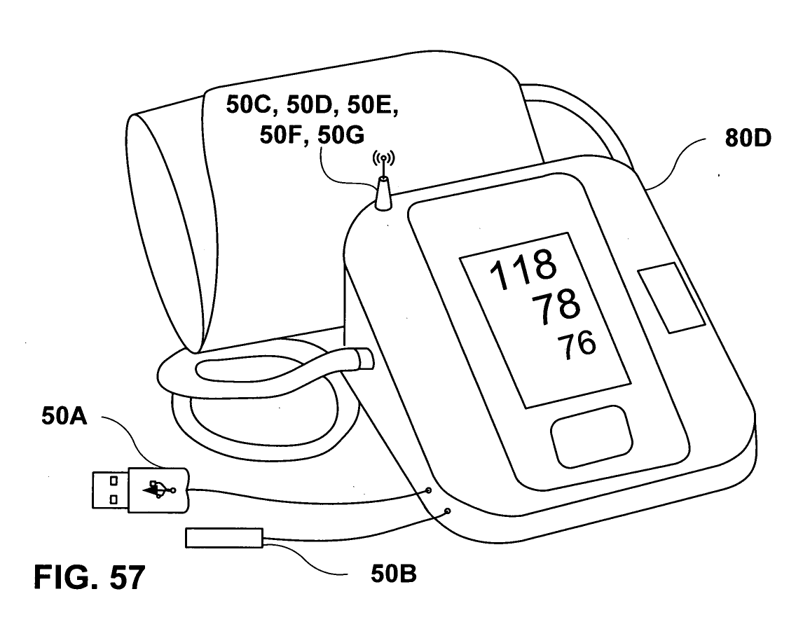

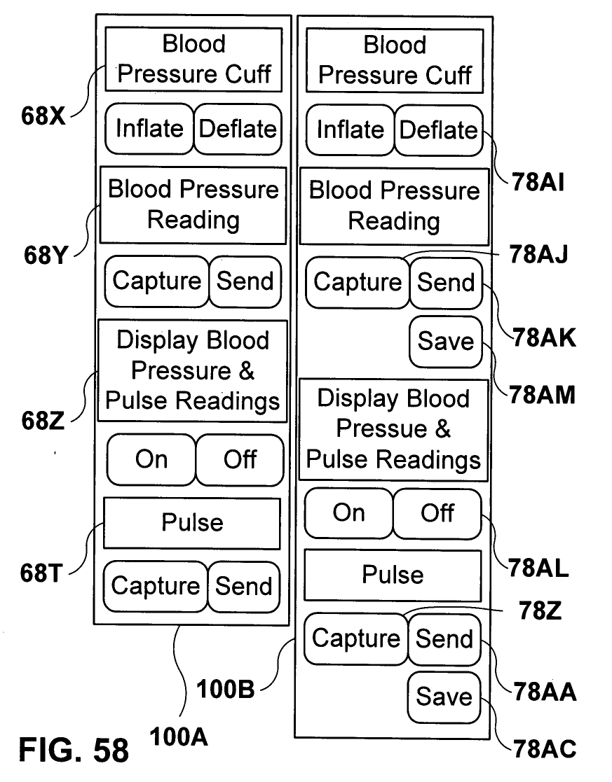

The fourth data device 80 is a blood pressure measurement device, such as a cuff 80D. Numbers of blood pressure cuffs 80D are commercially available from Health-O-Meter®, Hitachi®, Lumiscope®, Mabis®, Microlife®, Omron®, Oregon Scientific®, Panasonic®, Samsung® and others. A preferred embodiment of a blood pressure cuff 80D to be deployed in the Personal Healthcare Assistant 10A is shown in FIG. 57, and includes a connection 50 to a device or terminal 16. The embodiment shown in FIG. 57 includes one or more wired connections, USB 50A and FireWire 50B, and one or more wireless connections, Bluetooth 50C, WiFi 50D, UWB 50E, ZigBee 50F and WiMax 50G.

For a diagnostic device 16A to receive the blood pressure 68AA and pulse readings 68V from the blood pressure cuff 80D, it must have embedded software 18A that recognizes that digital blood pressure 68AA and pulse 68V readings are being sent to the diagnostic device 16A. One embodiment of the embedded software 18A allows the blood pressure and pulse readings to be displayed on the screen 38 of the diagnostic device 16A. Having received the blood pressure 68AA and 68V pulse readings from the blood pressure cuff 80D, there must be additional software 18A to forward the blood pressure 68AA and pulse 68V readings to the diagnostic, display and control means 24A used by a healthcare provider 20C.

Similarly, the diagnostic, display and control software application 26A deployed on a diagnostic, display and control means 24A used by a healthcare provider 20C has to have the ability to display the blood pressure 68AA and pulse 68V readings received from the diagnostic device 16A. The healthcare provider 20C must have the ability to take or retake the blood pressure 68AA and pulse 68V readings, as well as to determine whether to display the blood pressure 68AA and pulse 68V readings on the diagnostic device 16A.

FIG. 58 shows a fourth embodiment of a functional block diagram 100 of embedded software 18A that enables remote functional control of the data devices 80 for the diagnostic means 16A as well as a software application 26A that enables remote functional control of the data devices 80 connected to the diagnostic means 16A. The functions shown in 100A are deployed as a component of 18A, the software embedded in a diagnostic means 16A; those in 100B as a component of the diagnostic, display and control software application 26A.

The functions 68 embedded in a diagnostic device 16A for controlling the data device 80 the blood pressure cuff 80D include, but are not limited to:
Inflate or deflate 68Z the blood pressure cuff 80D;
Capture and send the blood pressure reading 68AA;
Capture and send the pulse reading 68V; and
Turn the blood pressure and pulse readings display 68AB on or off.

The functional control means 78 in the diagnostic, display and control software application 26A for remotely controlling the blood pressure cuff 80D via a diagnostic device 16A include, but are not limited to:
A button 78AK for inflating and deflating 68Z the blood pressure cuff 80D;
A button 78AL for capturing or recapturing the blood pressure reading 68AA;
A button 78AM for sending the blood pressure reading 68AA to a Healthcare provider 20C;
A button 78AB for capturing or recapturing the pulse reading 68V;
A button 78AC for sending the pulse reading 68V to a healthcare provider 20C; and
An “off” button and an “on” button 78AN for controlling the display 68AB of the blood pressure 68AA and pulse 68V readings.

An additional functional control means 78AO allows the healthcare provider 20C to save the blood pressure reading 68AA to the patient's 12A Electronic Medical Record (EMR) 60. The software application 26A automatically tags the blood pressure reading 68AA with the date 56A and current time 56B.

An additional functional control means 78AE allows the healthcare provider 20C to save the pulse reading 68V to the patient's 12A Electronic Medical Record (EMR) 60. The software application 26A automatically tags the pulse reading 68V with the date 56A and current time 56B.

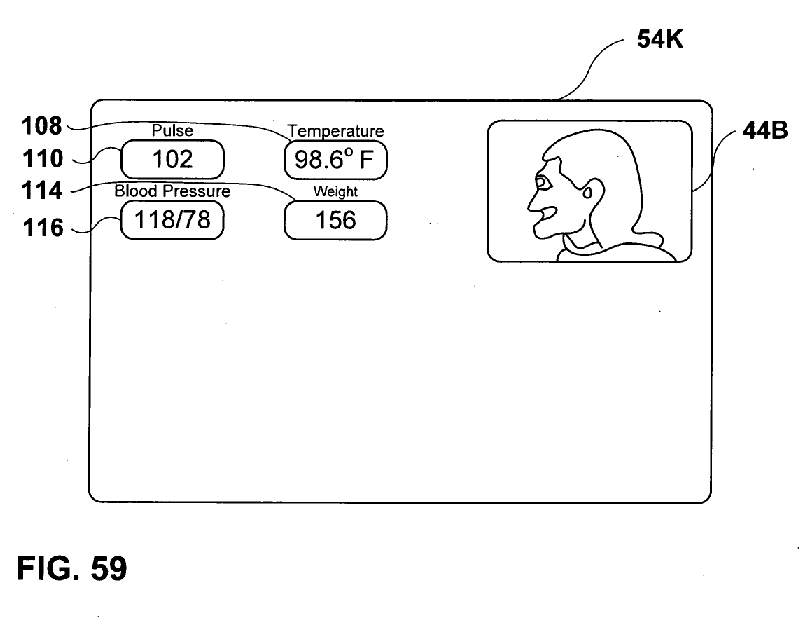

The pulse reading 68V appears in window 110 on the healthcare provider's 20C diagnostic, display and control means 24A as shown in FIG. 59.

The blood pressure reading 68AA appears in window 1116 on the healthcare provider's 20C diagnostic, display and control means 24A as shown in FIG. 59.

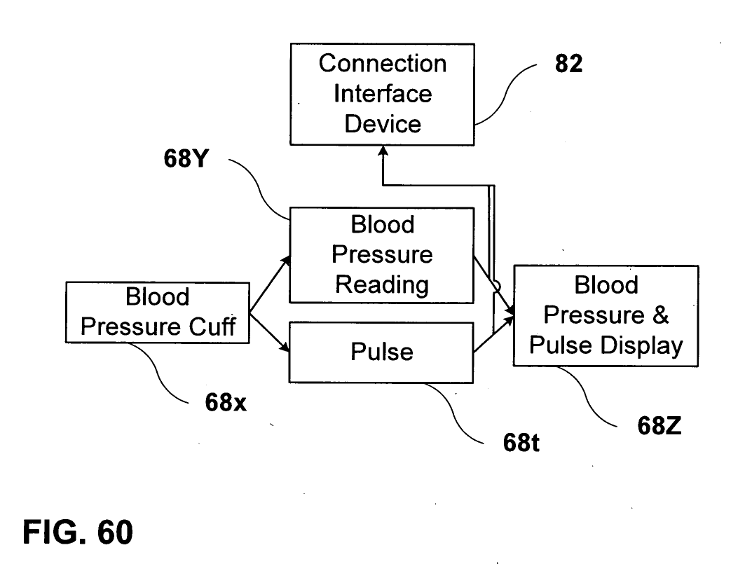

FIG. 60 shows the pulse 68V and the blood pressure 68AA reading fed to the connection interface device 82 for connection 50 to the device or terminal 16, including via the audio interface 92H.

### Oximeter

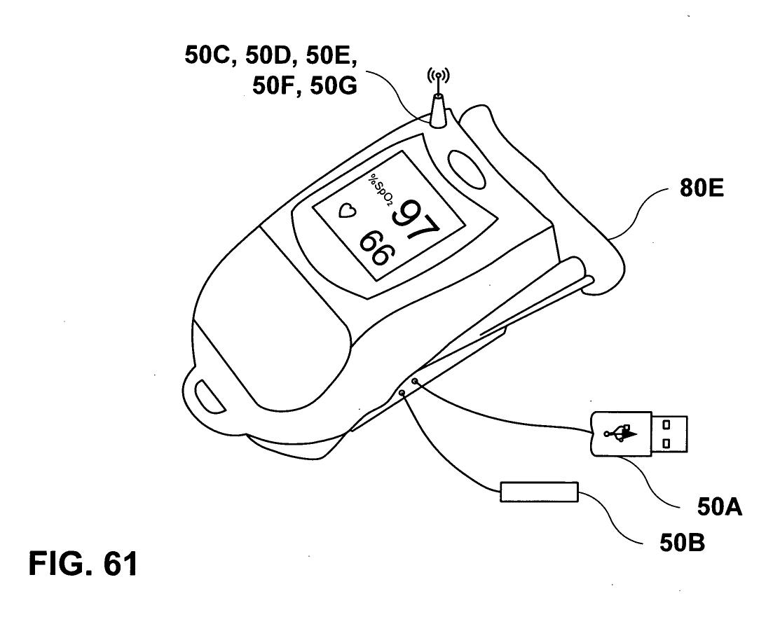

The fifth data device 80 is a device which measures levels of oxygen in the blood, such as an oximeter 80E. Numbers of oximeters 80E are commercially available from BCI®, Criticare®, INVOS®, Nonin Medical®, Smiths Medical PM Inc.®, SPO®, Turner Medical® and others. The Nonin Medical Inc. Avant™ 6800 Digital Pulse Oximetry System sends pulse rate data from a wrist-worn sensor to a monitor via Bluetooth. A preferred embodiment of an oximeter 80E to be deployed in the Personal Healthcare Assistant 10A is shown in FIG. 61, and includes a connection 50 to a device or terminal 16. The embodiment shown in FIG. 61 includes one or more wired connections, USB 50A and FireWire 50B, and one or more wireless connections, Bluetooth 50C, WiFi 50D, UWB 50E, ZigBee 50F and WiMax 50G.

For a diagnostic device 16A to receive the reading of the percent of hemoglobin that is saturated with oxygen 68AD and pulse 68V reading from the oximeter 80E, it must have embedded software 18A that recognizes that the reading of the percent of hemoglobin that is saturated with oxygen 68AD and pulse 68V readings are being sent to the diagnostic device 16A. One embodiment of the embedded software 18A allows the reading of the percent of hemoglobin that is saturated with oxygen 68AD and pulse 68V readings to be displayed on the screen 38 of the diagnostic device 16A. Having received the reading of the percent of hemoglobin that is saturated with oxygen 68AD and pulse 68V readings from the oximeter 80E, there must be additional software 18A to forward the reading of the percent of hemoglobin that is saturated with oxygen 68AD and pulse 68V readings to the diagnostic, display and control means 24A used by a healthcare provider 20C.

Similarly, the diagnostic, display and control software application 26A deployed on a diagnostic, display and control means 24A used by a healthcare provider 20C has to have the ability to display the reading of the percent of hemoglobin that is saturated with oxygen 68AD and pulse 68V readings received from the diagnostic device 16A. The healthcare provider 20C must have the ability to take or retake the reading of the percent of hemoglobin that is saturated with oxygen 68AD and the pulse reading 68V, as well as to determine whether to display the readings on the diagnostic device 16A.

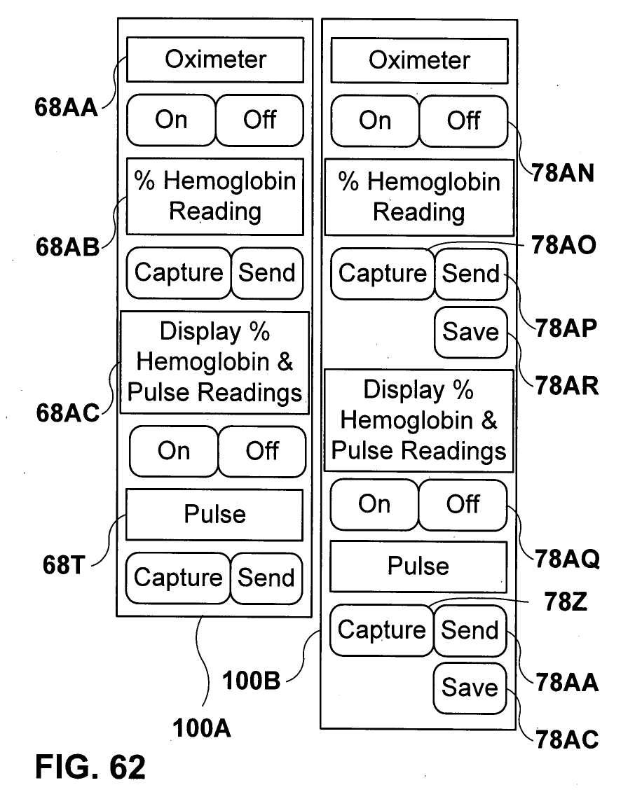

FIG. 62 shows a fifth embodiment of a functional block diagram 100 of embedded software 18A that enables remote functional control of the data devices 80 for the diagnostic means 16A as well as a software application 26A that enables remote functional control of the data devices 80 connected to the diagnostic means 16A. The functions shown in 100A are deployed as a component of 18A, the software embedded in a diagnostic means 16A; those in 100B as a component of the diagnostic, display and control software application 26A.

The functions 68 embedded in a diagnostic device 16A for controlling the data device 80 the oximeter 80E include, but are not limited to:
Turn the oximeter 80E on or off 68AC;
Capture and send the reading of the percent of hemoglobin that is saturated with oxygen 68AD;
Capture and send the pulse reading 68V; and
Turn the reading of the percent of hemoglobin that is saturated with oxygen and pulse reading display 68AE on or off.

The functional control means 78 in the diagnostic, display and control software application 26A for remotely controlling the oximeter 80E via a diagnostic device 16A include, but are not limited to:
An “off” button and an “on” button 78AP for controlling 68AD the oximeter 80E;
A button 78AQ for capturing or recapturing the reading of the percent of hemoglobin that is saturated with oxygen 68AD;
A button 78AR for sending the reading of the percent of hemoglobin that is saturated with oxygen 68AD to a healthcare provider 20C;
A button 78AB for capturing or recapturing the pulse reading 68V;
A button 78AC for sending the pulse reading 68V to a healthcare provider 20C; and
An “off” button and an “on” button 78AS for controlling the display 68AE of the reading of the percent of hemoglobin that is saturated with oxygen 68AD and pulse 68V reading.

An additional functional control means 78AT allows the healthcare provider 20C to save the reading of the percent of hemoglobin that is saturated with oxygen 68AD to the patient's 12A Electronic Medical Record (EMR) 60. The software application 26A automatically tags the reading of the percent of hemoglobin that is saturated with oxygen 68AD with the date 56A and current time 56B.

An additional functional control means 78AE allows the healthcare provider 20C to save the pulse reading 68V to the patient's 12A Electronic Medical Record (EMR) 60. The software application 26A automatically tags the pulse reading 68V with the date 56A and current time 56B.

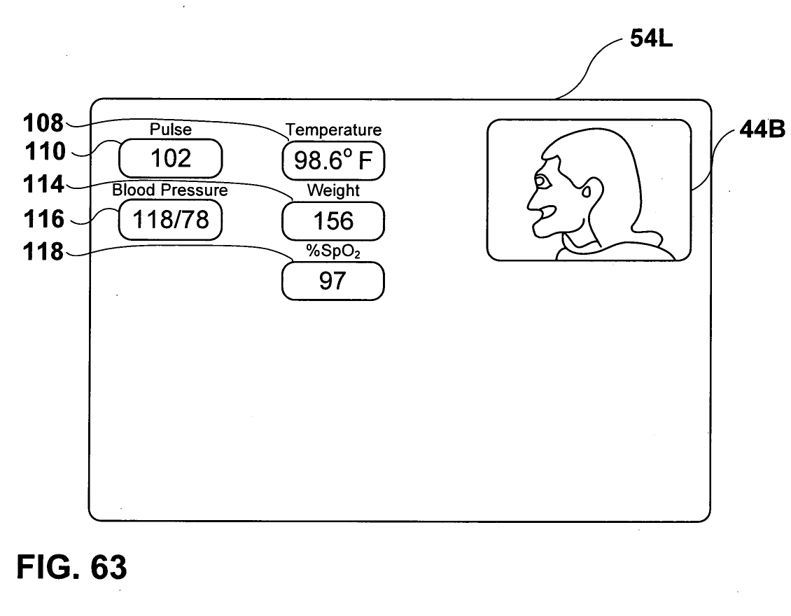

The pulse reading 68V appears in window 110 on the healthcare provider's 20C diagnostic, display and control means 24A as shown in FIG. 63.

The reading of the percent of hemoglobin that is saturated with oxygen 68AD appears in window 118 on the healthcare provider's 20C diagnostic, display and control means 24A as shown in FIG. 63.

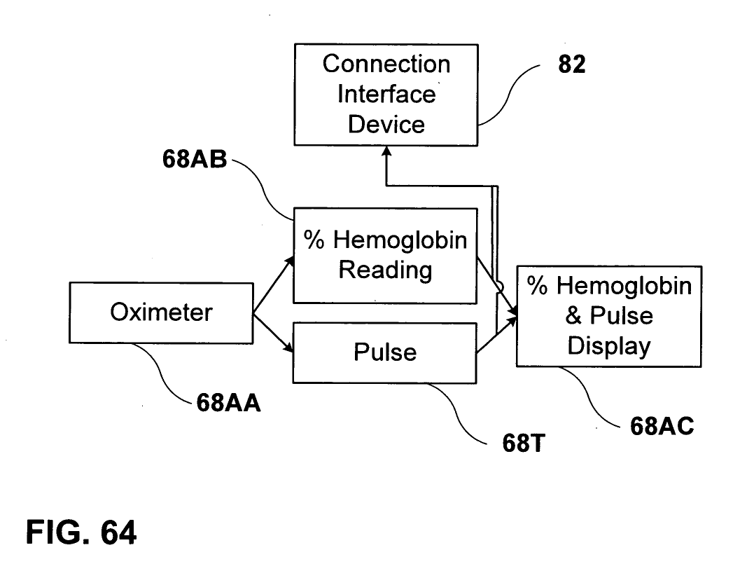

FIG. 64 shows the pulse 68V and the percent of hemoglobin that is saturated with oxygen 68AD fed to the connection interface device 82 for connection 50 to the device or terminal 16, including via the audio interface 92H.

### Electrocardiogram

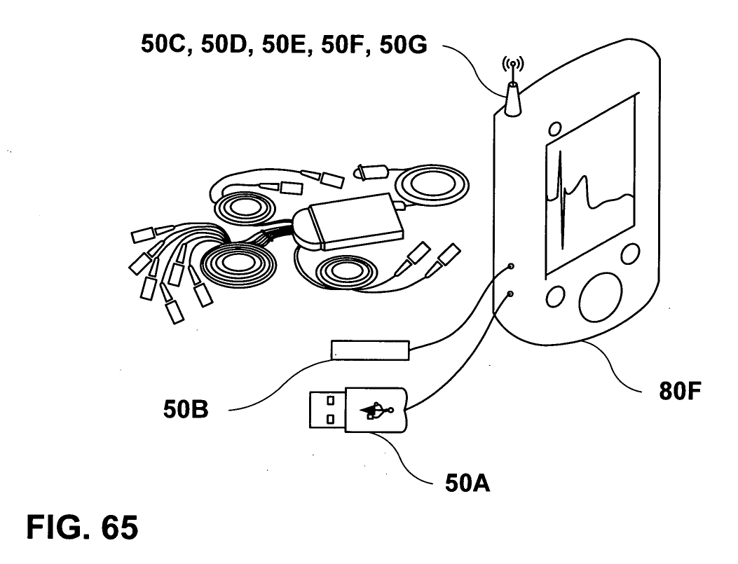

The sixth data device 80 is a device for obtaining an electrocardiograph, such as an electrocardiogram unit 80F. Numbers of electrocardiogram units 80F are commercially available from Biolog®, Bionet®, Burdich®, Brentwood®, Cardioline®, GE Marquette®, Midmark®, Nihon Kohden®, Phillips®, QRS®, Schiller America®, WelchAllyn® and others. A preferred embodiment of an electrocardiogram unit 80F to be deployed in the Personal Healthcare Assistant 10A is shown in FIG. 65, and includes a connection 50 to a device or terminal 16. The embodiment shown in FIG. 65 includes one or more wired connections, USB 50A and FireWire 50B, and one or more wireless connections, Bluetooth 50C, WiFi 50D, UWB 50E, ZigBee 50F and WiMax 50G.

For a diagnostic device 16A to receive the electrocardiogram 68AG and pulse 68V reading from the electrocardiogram unit 80F, it must have embedded software 18A that recognizes that the electrocardiogram 68AG and pulse 68V reading are being sent to the diagnostic device 16A. Having received the electrocardiogram 68AG and pulse 68V reading from the electrocardiogram unit 80F, there must be additional software 18A to forward the electrocardiogram 68AG and pulse 68V readings to the diagnostic, display and control means 24A used by a healthcare provider 20C.

Similarly, the diagnostic, display and control software application 26A deployed on a diagnostic, display and control means 24A used by a healthcare provider 20C has to have the ability to display the electrocardiogram 68AG and pulse 68V readings received from the diagnostic device 16A. The healthcare provider 20C must have the ability to take or retake the electrocardiogram 68AG and pulse 68V readings.

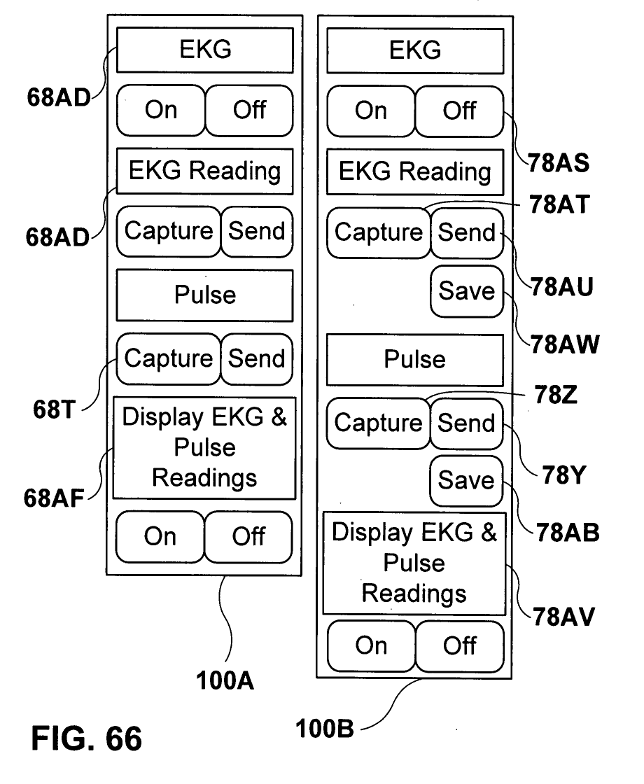

FIG. 66 shows a sixth embodiment of a functional block diagram 100 of embedded software 18A that enables remote functional control of the data devices 80 for the diagnostic means 16A as well as a software application 26A that enables remote functional control of the data devices 80 connected to the diagnostic means 16A. The functions shown in 100A are deployed as a component of 18A, the software embedded in a diagnostic means 16A; those in 100B as a component of the diagnostic, display and control software application 26A.

The functions 68 embedded in a diagnostic device 16A for controlling the data device 80 the electrocardiogram 80F include, but are not limited to:
Turn the electrocardiogram unit 80F on or off 68AF;
Capture and send the electrocardiogram reading 68AG;
Capture and send the pulse reading 68V; and
Turn the electrocardiogram 68AG and pulse 68V reading display 68AH on or off.

The functional control means 78 in the diagnostic, display and control software application 26A for remotely controlling the electrocardiogram unit 80F via a diagnostic device 16A include, but are not limited to:
An “off” button and an “on” button 78AU for controlling 68AD the electrocardiogram unit 80F;
A button 78AV for capturing or recapturing the electrocardiogram reading 68AG;
A button 78AW for sending the electrocardiogram reading 68AG to a healthcare provider 20C;
A button 78AB for capturing or recapturing the pulse reading 68V;
A button 78AC for sending the pulse reading 68V to a healthcare provider 20C; and
An “off” button and an “on” button 78AX for controlling the display 68AH of the electrocardiogram 68AG and pulse 68V reading.

An additional functional control means 78AY allows the healthcare provider 20C to save the electrocardiogram reading 68AG to the patient's 12A Electronic Medical Record (EMR) 60. The software application 26A automatically tags the electrocardiogram 68AG with the date 56A and current time 56B.

An additional functional control means 78AE allows the healthcare provider 20C to save the pulse reading 68V to the patient's 12A Electronic Medical Record (EMR) 60. The software application 26A automatically tags the pulse reading 68V with the date 56A and current time 56B.

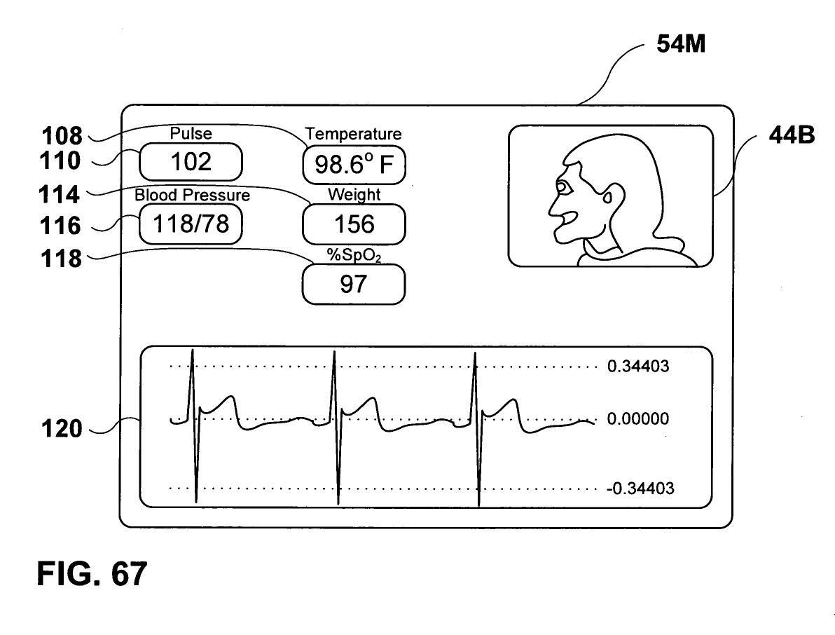

The pulse reading 68V appears in window 110 on the healthcare provider's 20C diagnostic, display and control means 24A as shown in FIG. 67.

The electrocardiogram 68AG appears in window 120 on the healthcare provider's 20C diagnostic, display and control means 24A as shown in FIG. 67.

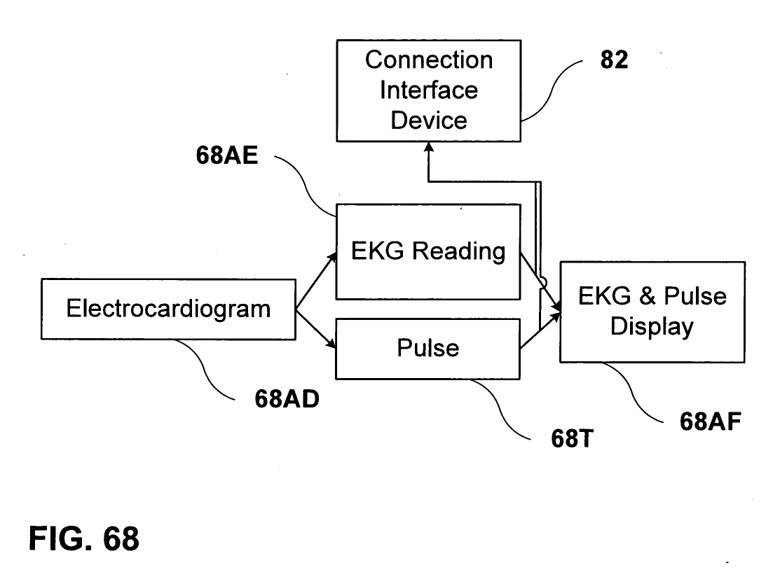

FIG. 68 shows the pulse 68V and the electrocardiogram 68AG fed to the connection interface device 82 for connection 50 to the device or terminal 16, including via the audio interface 92H.

### Glucose Meter

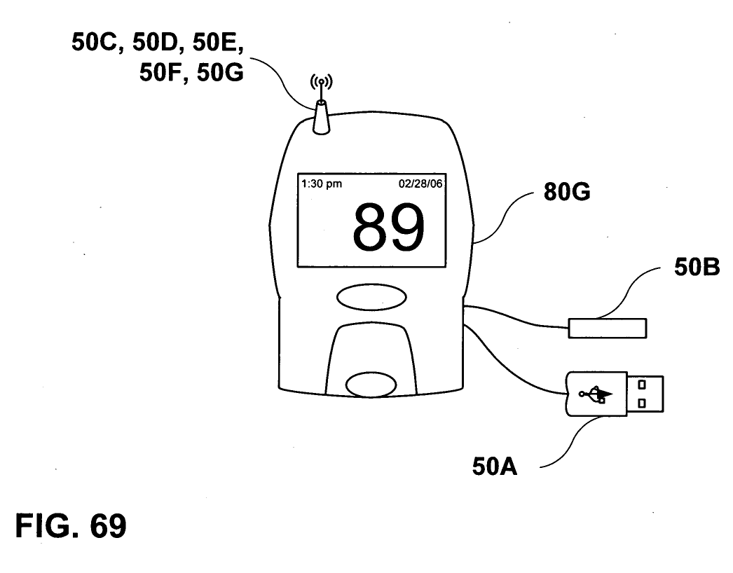

The seventh data device 80 is a device for measuring the glucose level in the blood, such as a glucose meter 80G. Numbers of glucose meters 80G are commercially available from Ascensia®, BD Logic®, Home Diagnostics, Inc.®, Hypoguard®, LifeScan®, MediSense®, Roche Diagnostics®, SpectRx, Inc.® and others. A preferred embodiment of a glucose meter 80G to be deployed in the Personal Healthcare Assistant 10A is shown in FIG. 69, and includes a connection 50 to a device or terminal 16. The embodiment shown in FIG. 69 includes one or more wired connections, USB 50A and FireWire 50B, and one or more wireless connections, Bluetooth 50C, WiFi 50D, UWB 50E, ZigBee 50F and WiMax 50G.

For a diagnostic device 16A to receive the glucose reading 68AJ from the meter 80G, it must have embedded software 18A that recognizes that a glucose reading 68AJ is being sent to the diagnostic device 16A. One embodiment of the embedded software 18A allows the glucose reading 68AJ to be displayed on the screen 38 of the diagnostic device 16A. Having received the glucose reading 68AJ from the meter 80G, there must be additional software 18A to forward the glucose reading 68AJ to the diagnostic, display and control means 24A used by a healthcare provider 20C.

Similarly, the diagnostic, display and control software application 26A deployed on a diagnostic, display and control means 24A used by a healthcare provider 20C has to have the ability to display the glucose reading 68AJ received from the diagnostic device 16A. The healthcare provider 20C must have the ability to take or retake the glucose reading 68AJ, as well as to determine whether to display the glucose reading 68AJ on the diagnostic device 16A.

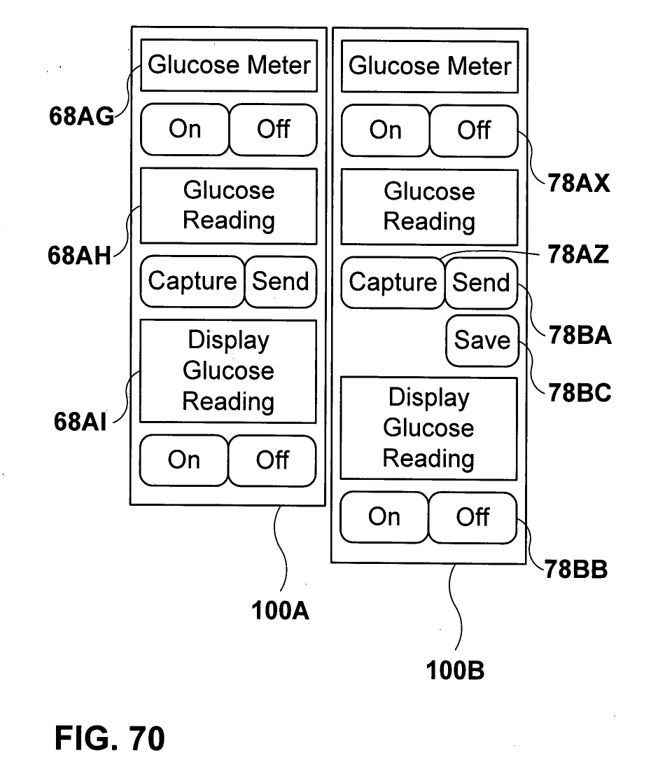

FIG. 70 shows a seventh embodiment of a functional block diagram 100 of embedded software 18A that enables remote functional control of the data devices 80 for the diagnostic means 16A as well as a software application 26A that enables remote functional control of the data devices 80 connected to the diagnostic means 16A. The functions shown in 100A are deployed as a component of 18A, the software embedded in a diagnostic means 16A; those in 100B as a component of the diagnostic, display and control software application 26A.

The functions 68 embedded in a diagnostic device 16A for controlling the data device 80 the glucose meter 80F include, but are not limited to:
Turn the glucose meter 80G on or off 68AI;
Capture and send the glucose reading 68AJ; and
Turn the glucose reading display 68AK on or off.

The functional control means 78 in the diagnostic, display and control software application 26A for remotely controlling the glucose meter 80G via a diagnostic device 16A include, but are not limited to:
An “off” button and an “on” button 78AZ for controlling 68AI the glucose meter 80G;
A button 78BA for capturing or recapturing the glucose reading 68AJ;
A button 78BB for sending the glucose reading 68AJ to a healthcare provider 20C; and
An “off” button and an “on” button 78BC for controlling the display 68AK of the glucose reading 68AJ.

An additional functional control means 78BD allows the healthcare provider 20C to save the glucose reading 68AJ to the patient's 12A Electronic Medical Record (EMR) 60. The software application 26A automatically tags the glucose reading 68AJ with the date 56A and current time 56B.

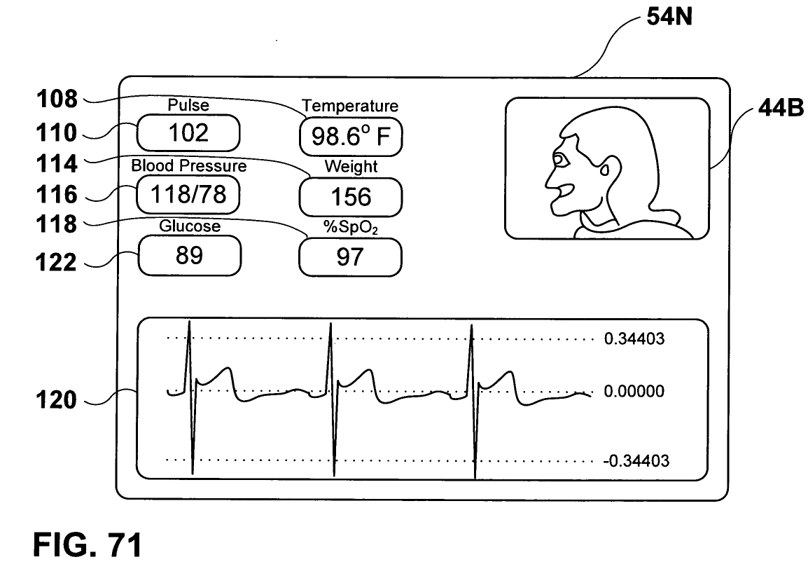

The glucose reading 68AJ appears in window 122 on the healthcare provider's 20C diagnostic, display and control means 24A as shown in FIG. 71.

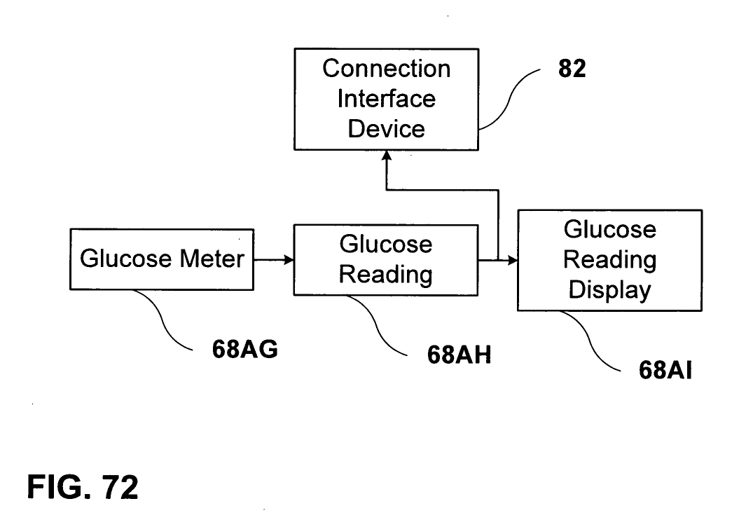

FIG. 72 shows the glucose reading 68AJ fed to the connection interface device 82 for connection 50 to the device or terminal 16, including via the audio interface 92H.

### Otoscope

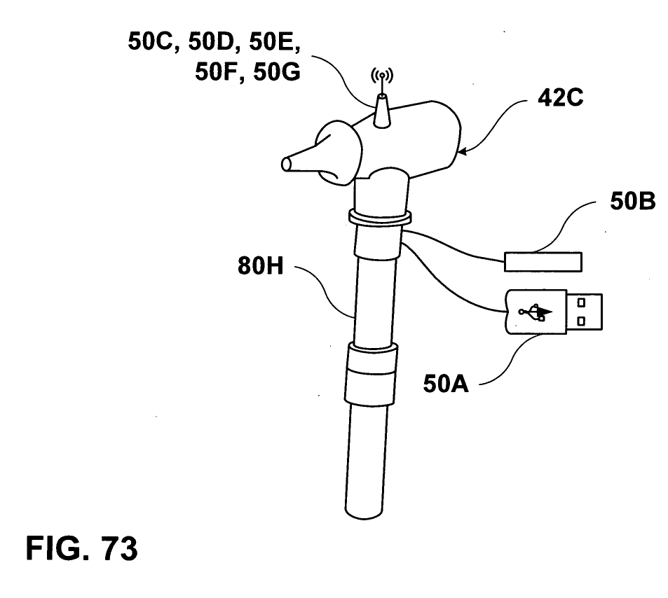

An eighth data device 80 is an otoscope 80H. An otoscope 80H is used to examine the ears, nose, and mouth. It contains a light and a magnifying lens. Numbers of otoscopes 80H are commercially available from American Diagnostic Corporation (ADC)®, Dr. Mom®, Heine®, Riester®, WelchAllyn® and others. A preferred embodiment of an otoscope 80H to be deployed in the Personal Healthcare Assistant 10A is shown in FIG. 73, and includes a connection 50 to a device or terminal 16. The embodiment shown in FIG. 73 includes one or more wired connections, USB 50A and FireWire 5BF, and one or more wireless connections, Bluetooth 50C, WiFi 50D, UWB 50E, ZigBee 50F and WiMax 50G.

An otoscope is basically a visual aid to a healthcare provider 20C who has physical access to a patient or potential patient 12A. A camera 42C must be added to the otoscope 80H for it to be deployed in the Personal Healthcare Assistant 10. The otoscope 80H must also have an interface that captures the images 68G and makes those images available to the connection 50 to the device or terminal 16.

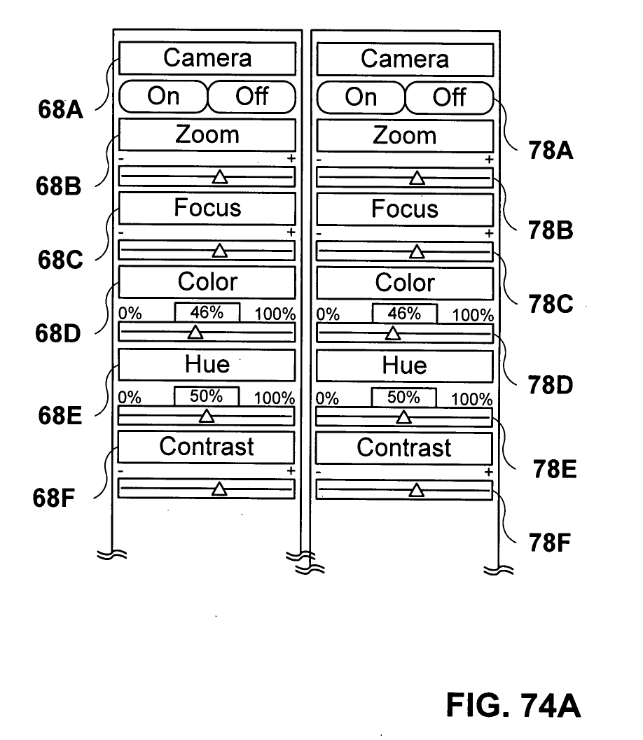

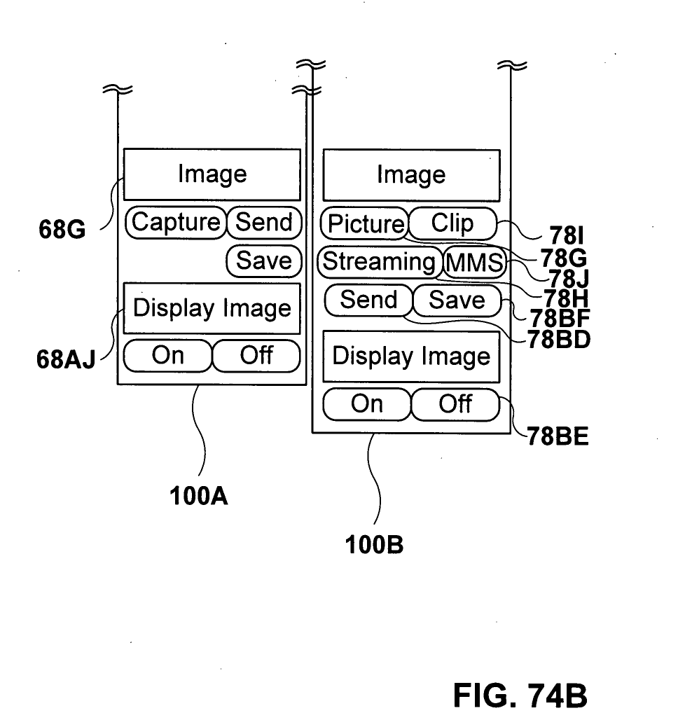

FIG. 74 shows a eighth embodiment of a functional block diagram 100 of embedded software 18A that enables remote functional control of the data devices 80 for the diagnostic means 16A as well as a software application 26A that enables remote functional control of the data devices 80 connected to the diagnostic means 16A. The functions shown in 100A are deployed as a component of 18A, the software embedded in a diagnostic means 16A; those in 100B as a component of the diagnostic, display and control software application 26A.

The functions 68 embedded in a diagnostic device 16A for controlling the data device 80 the otoscope 80H include, but are not limited to:
Turn the camera 80H on or off 68A;
Zoom 68B the camera 80H;
Focus 68C the camera 80H;
Adjust the camera 80H color 68D;
Adjust the camera 80H hue 68E;
Adjust the camera 80H contrast 68F;
Capture an image 68G; and
Turn the camera 80H display 68AL on or off.

The functional control means 78 in the diagnostic, display and control software application 26A for remotely controlling the otoscope 80H via a diagnostic device 16A include, but are not limited to:
An “off” button and an “on” button 78A for controlling 68A the camera 80H;
A slider 78B that adjusts the zoom 68B from zero percent (0%) to one hundred percent (84%);
A slider 78C that adjusts the camera focus 68C from minus (−) to plus
A slider 78D that adjusts the color 68D from zero percent (0%) to one hundred percent (84%);
A slider 78E that adjusts the hue 68E from zero percent (0%) to one hundred percent (84%);
A slider 78F that adjusts the contrast 68F from minus (−) to plus (+);
A button 78G for capturing 68G the image 44E as a picture 44E1;
A button 78H for capturing 68G the image 44E as streaming video 44E2;
A button 78I for capturing 68G the image 44E as a video clip 44EB3;
A button 78J for capturing 68G the image 44E as a multi-media message (MMS) 44E4;
A button 78BE for sending 68G the image 44E; and
An “off” button and an “on” button 78BF for controlling the display 68AJ of the camera 80H.

An additional functional control means 78BG allows the healthcare provider 20C to save the image 44E to the patient's 12A Electronic Medical Record (EMR) 60. The software application 26A automatically tags the image 44E with the date 56A and current time 56B.

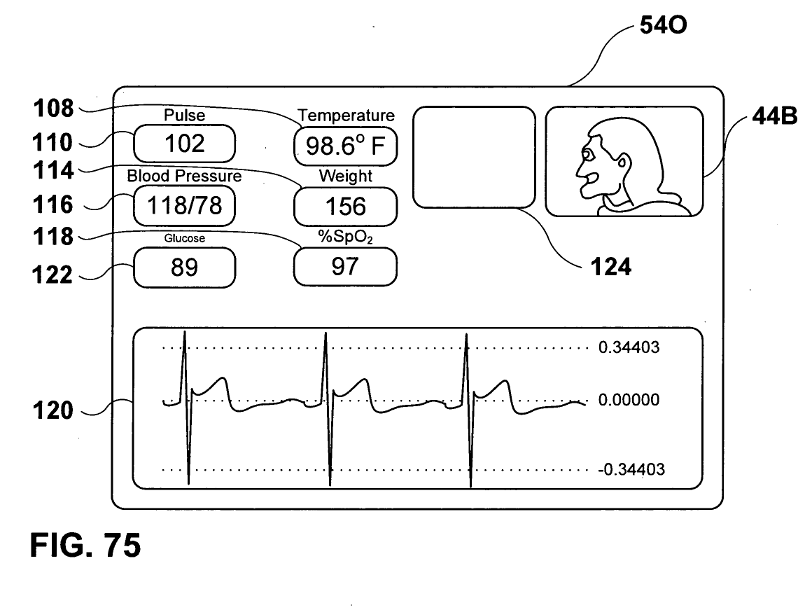

The image 44E appears in window 124 on the healthcare provider's 20C diagnostic, display and control means 24A as shown in FIG. 75.

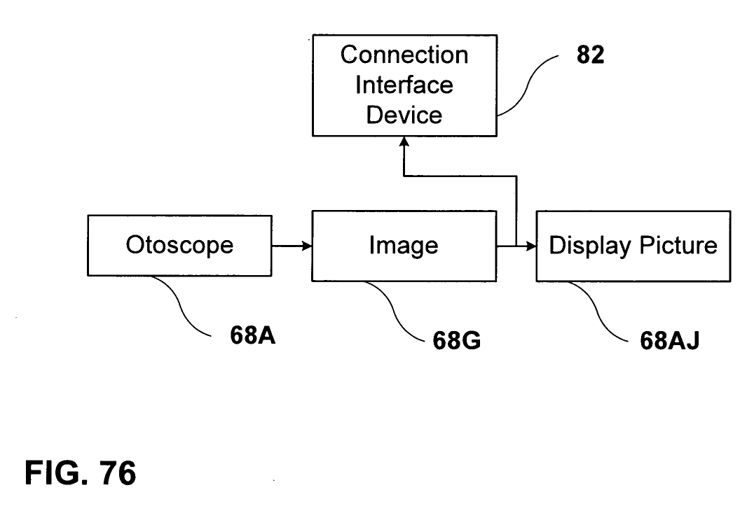

FIG. 76 shows the image 68G fed to the connection interface device 82 for connection 50 to the device or terminal 16.

### Ultrasound

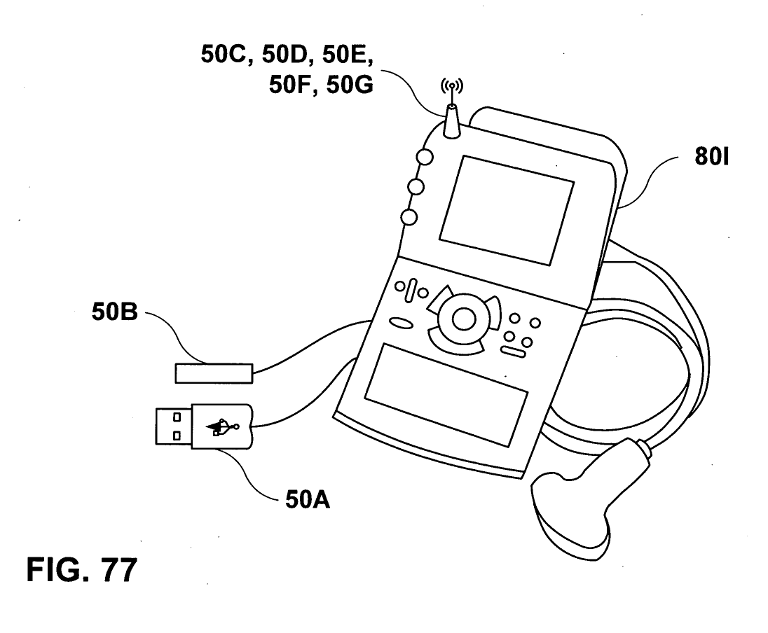

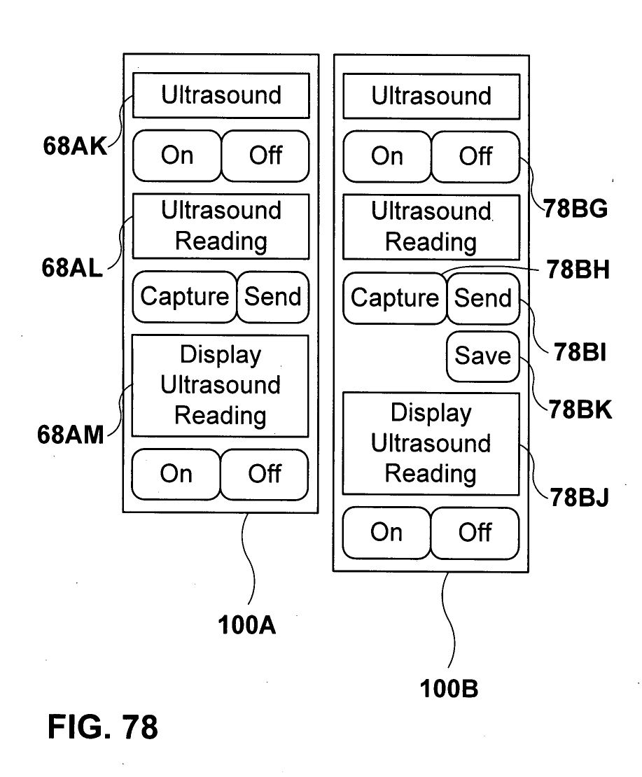

The ninth data device 80 is an ultrasound unit 80I. Numbers of ultrasound units 80I are commercially available from Amrex®, Intelect®, GE Logiq®, Koality®, Mettler®, Siemens Acuson® and others. A preferred embodiment of an ultrasound unit 80I to be deployed in the Personal Healthcare Assistant 10A is shown in FIG. 77, and includes a connection 50 to a device or terminal 16. The embodiment shown in FIG. 77 includes one or more wired connections, USB 50A and FireWire 50B, and one or more wireless connections, Bluetooth 50C, WiFi 50D, UWB 50E, ZigBee 50F and WiMax 50G.

For a diagnostic device 16A to receive the ultrasound readings 68AN from the ultrasound unit 80I, it must have embedded software 18A that recognizes that the ultrasound readings 68AN are being sent to the diagnostic device 16A. Having received the ultrasound readings 68AN from the ultrasound unit 80I, there must be additional software 18A to forward the ultrasound readings 68AN to the diagnostic, display and control means 24 used by a healthcare provider 20C.

Similarly, the diagnostic, display and control software application 26A deployed on a diagnostic, display and control means 24A used by a healthcare provider 20C has to have the ability to display the ultrasound readings 68AN received from the diagnostic device 16A. The healthcare provider 20C must have the ability to take or retake the ultrasound readings 68AN.

FIG. 78 shows a ninth embodiment of a functional block diagram 100 of embedded software 18A that enables remote functional control of the data devices 80 for the diagnostic means 16A as well as a software application 26A that enables remote functional control of the data devices 80 connected to the diagnostic means 16A. The functions shown in 100A are deployed as a component of 18A, the software embedded in a diagnostic means 16A; those in 100B as a component of the diagnostic, display and control software application 26A.

The functions 68 embedded in a diagnostic device 16A for controlling the data device 80 the ultrasound unit 80I include, but are not limited to:
Turn the ultrasound unit 80I on or off 68AM;
Capture and send the ultrasound reading 68AN; and
Turn the ultrasound display 68AO on or off.

The functional control means 78 in the diagnostic, display and control software application 26A for remotely controlling the ultrasound unit 80I via a diagnostic device 16A include, but are not limited to:
An “off” button and an “on” button 78BH for controlling 68AM the ultrasound unit 80I;
A button 78BI for capturing or recapturing the ultrasound readings 68AN;
A button 78BJ for sending the ultrasound readings 68AN to a healthcare provider 20C; and
An “off” button and an “on” button 78BK for controlling the display 68AO of the ultrasound 80I.

An additional functional control means 78BL allows the healthcare provider 20C to save the ultrasound readings 68AN to the patient's 12A Electronic Medical Record (EMR) 60. The software application 26A automatically tags the ultrasound readings 68AN with the date 76 and current time 80.

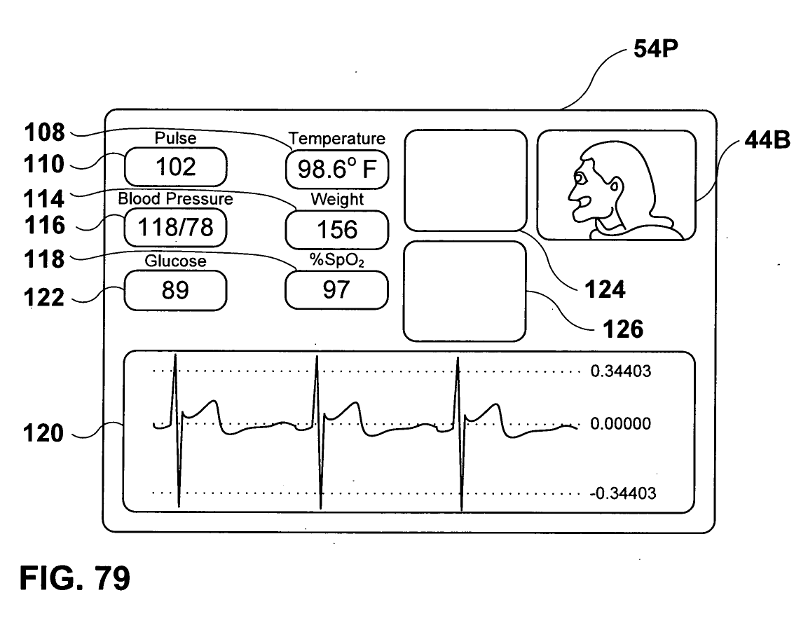

The ultrasound readings 68AN appears in window 126 on the healthcare provider's 20C diagnostic, display and control means 24A as shown in FIG. 79.

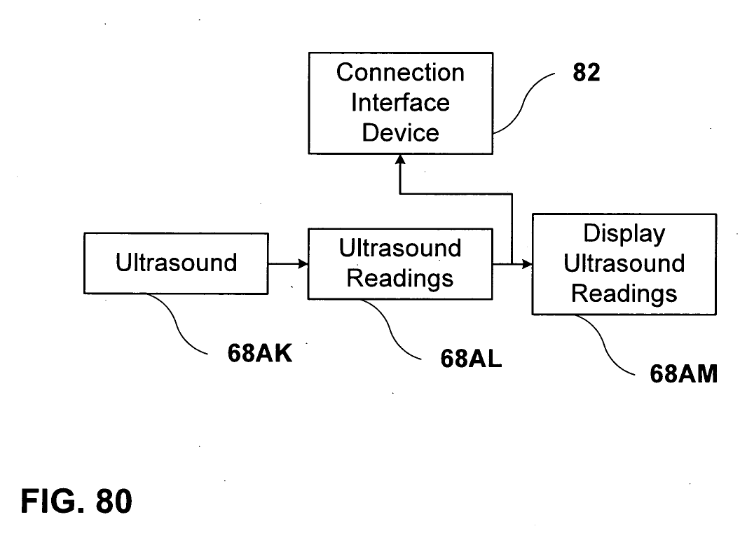

FIG. 80 shows the ultrasound readings 68AN fed to the connection interface device 82 for connection 50 to a device or terminal 16.

### Spirometer

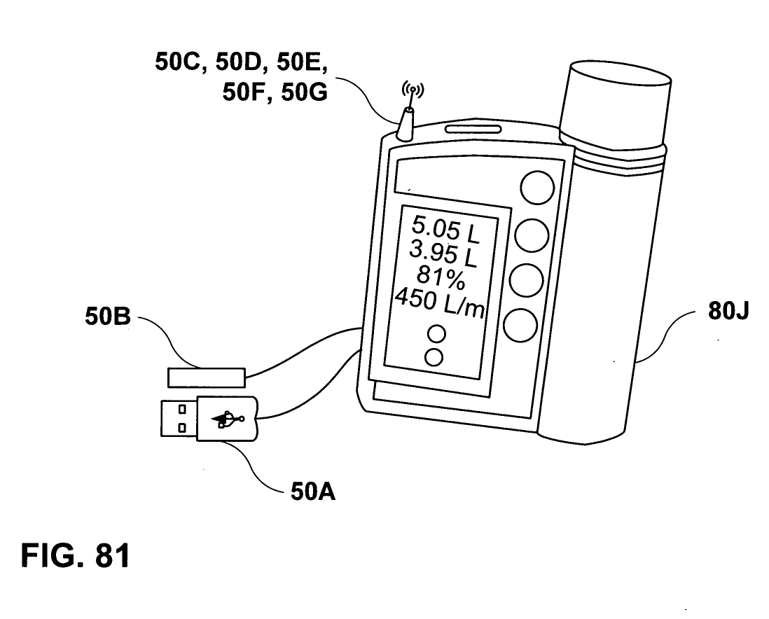

The tenth data device 80 is a spirometer 80J, which measures the volume and flow rate of inhaled and exhaled air. Numbers of spirometers 80J are commercially available from Jones Medical Instrument Co., Micro Medical, Ltd., Puritan-Bennett, QRS Diagnostic, LLC, Spirometrics Inc, Vitalograph, Welch Allyn and others. A preferred embodiment of an ultrasound unit 80J to be deployed in the Personal Healthcare Assistant 10A is shown in FIG. 81, and includes a connection 50 to a device or terminal 16. The embodiment shown in FIG. 81 includes one or more wired connections, USB 50A and FireWire 50B, and one or more wireless connections, Bluetooth 50C, WiFi 50D, UWB 50E, ZigBee 50F and WiMax 50G.

For a diagnostic device 16A to receive the spirometer readings 68AQ from the spirometer unit 80J, it must have embedded software 18A that recognizes that the spirometer readings 68AQ are being sent to the diagnostic device 16A. Having received the spirometer readings 68AQ from the spirometer unit 80J, there must be additional software 18A to forward the spirometer readings 68AQ to the diagnostic, display and control means 24 used by a healthcare provider 20C.

Similarly, the diagnostic, display and control software application 26A deployed on a diagnostic, display and control means 24A used by a healthcare provider 20C has to have the ability to display the spirometer readings 68AQ received from the diagnostic device 16A. The healthcare provider 20C must have the ability to take or retake the spirometer readings 68AQ.

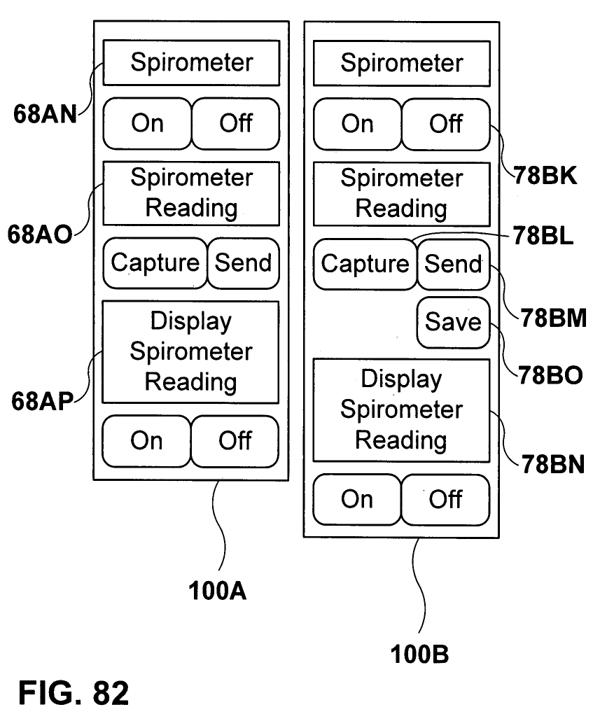

FIG. 82 shows a tenth embodiment of a functional block diagram 100 of embedded software 18A that enables remote functional control of the data devices 80 for the diagnostic means 16A as well as a software application 26A that enables remote functional control of the data devices 80 connected to the diagnostic means 16A. The functions shown in 100A are deployed as a component of 18A, the software embedded in a diagnostic means 16A; those in 100B as a component of the diagnostic, display and control software application 26A.

The functions 68 embedded in a diagnostic device 16A for controlling the data device 80 the spirometer unit 80J include, but are not limited to:
Turn the spirometer unit 80J on or off 68AP;
Capture and send the spirometer reading 68AQ; and
Turn the spirometer display 68AR on or off.

The functional control means 78 in the diagnostic, display and control software application 26A for remotely controlling the spirometer unit 80J via a diagnostic device 16A include, but are not limited to:
An “off” button and an “on” button 78BM for controlling 68AP the spirometer unit 80J;
A button 78BN for capturing or recapturing the spirometer readings 68AQ;
A button 78BO for sending the spirometer readings 68AQ to a healthcare provider 20C; and
An “off” button and an “on” button 78BP for controlling the display 68AR of the spirometer 80J.

An additional functional control means 78BQ allows the healthcare provider 20C to save the spirometer readings 68AQ to the patient's 12A Electronic Medical Record (EMR) 60. The software application 26A automatically tags the spirometer readings 68AQ with the date 56A and current time 56B.

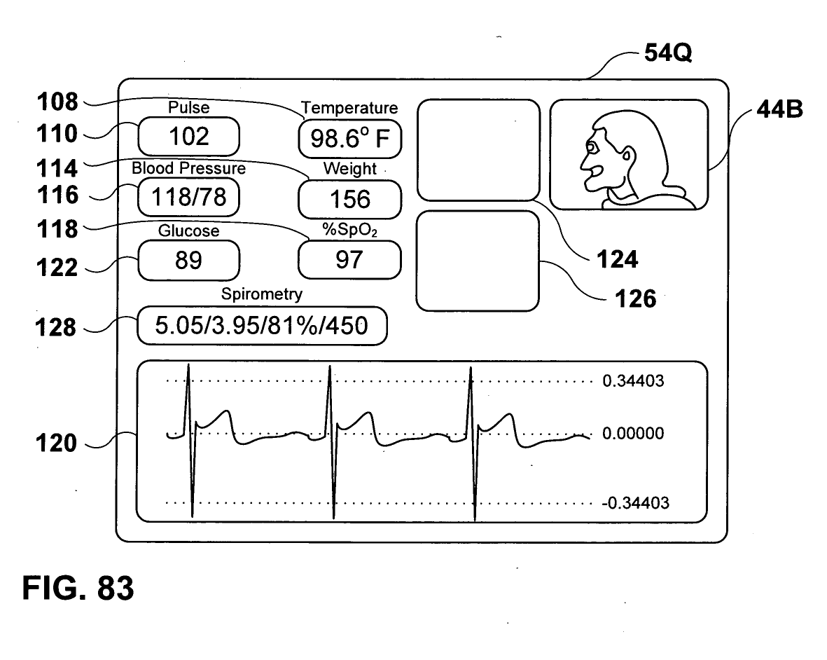

The spirometer readings 68AQ appear in window 128 on the healthcare provider's 20C diagnostic, display and control means 24A as shown in FIG. 83.

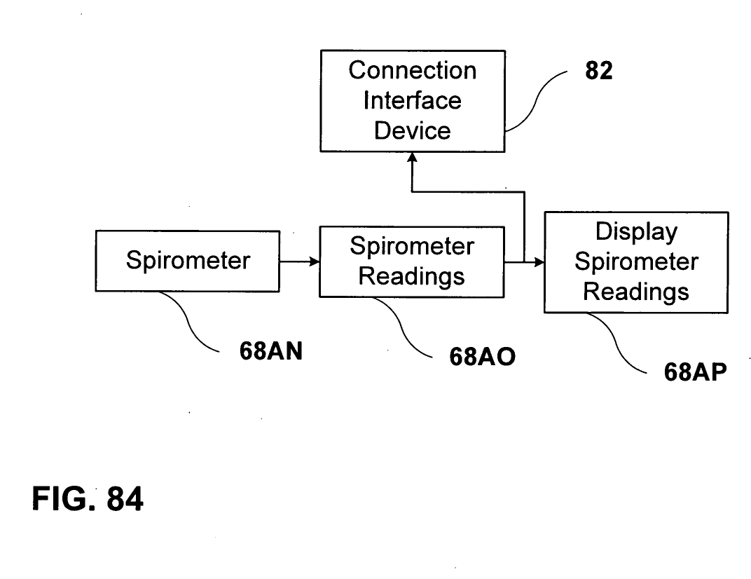

FIG. 84 shows the spirometer readings 68AQ fed to the connection interface device 82 for connection 50 to a device or terminal 16, including via the audio interface 92H.

### External Camera

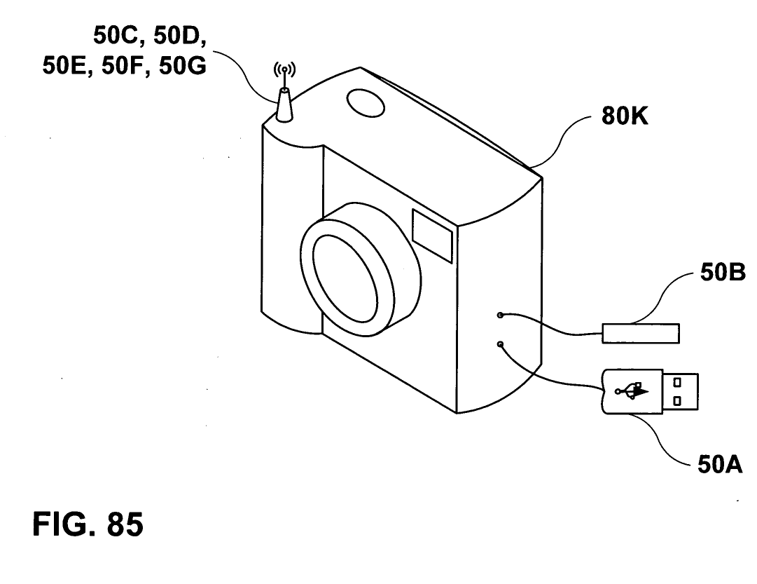

The eleventh data device 80 is a camera 80K that is not part of a diagnostic means 16A. A preferred embodiment of an external camera 80K to be deployed in the Personal Healthcare Assistant 10 is shown in FIG. 85, and includes a connection 50 to a device or terminal 16. The embodiment shown in FIG. 85 includes one or more wired connections, USB 50A and FireWire 50B, and one or more wireless connections, Bluetooth 50C, WiFi 50D, UWB 50E, ZigBee 50F and WiMax 50G.

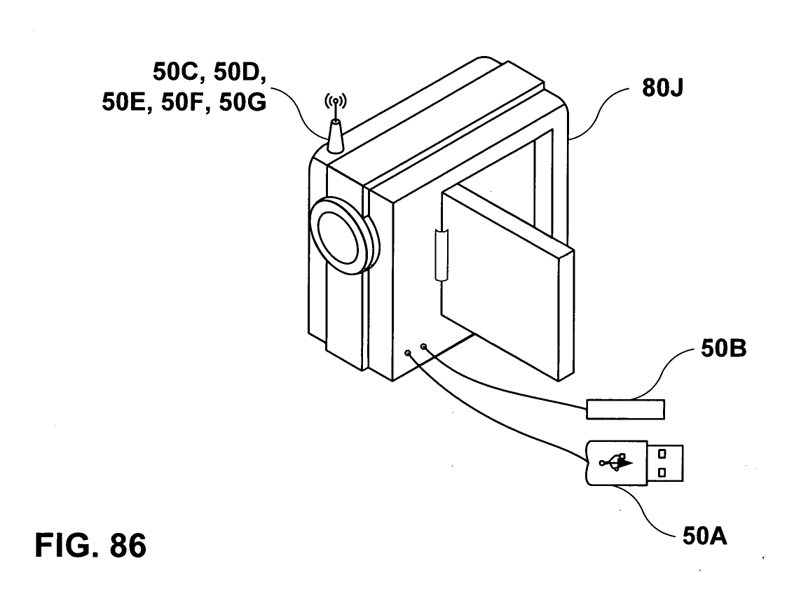

The specific embodiment of the external camera 80K shown in FIG. 85 is a digital camera. Modern digital cameras take pictures 44D1 as well as short videos 44D3. An alternative embodiment of the external camera 80K is a digital video recorder as shown in FIG. 86. Modern digital video cameras take videos 44D3 as well as pictures 44D1. The embodiment shown in FIG. 86 also includes one or more wired connections, USB 50A and FireWire 50B, and one or more wireless connections, Bluetooth 50C, WiFi 50D, UWB 50E, ZigBee 50F and WiMax 50G.

At present there are only few manufacturers of Bluetooth-enabled cameras, Concord Camera®, Panasonic®, Sony®, Sony Ericcson®; other manufacturers are expected to enter this market in the near future. Some manufacturers have WiFi-enabled cameras, 4xem®, Axis Communications®, BenQ®, Creative Labs®, D-Link®, Kodak®, Linksys®, Nikon®, Sony® and others. Additional manufacturers are expected to enter this market in the near future. It is expected that manufacturers will add UWB to cameras in the near future.

For a diagnostic device 16A to receive the image 68G from the external camera 80K, it must have embedded software 18A that recognizes that a camera image 68G is being sent to the diagnostic device 16A. One embodiment of the embedded software 18A allows the camera image 68G to be displayed on the screen 40A of the diagnostic device 16A. Having received the image 68G from the external camera 80K, there must be additional software 18A to forward the external camera image 68G to the diagnostic, display and control means 24A used by a healthcare provider 20C.

Similarly, the diagnostic, display and control software application 26A deployed on a diagnostic, display and control means 24A used by a healthcare provider 20C has to have the ability to display the external camera image 68G received from the diagnostic device 16A. The healthcare provider 20C must have the ability to take or retake the camera images 68G, as well as to determine whether to display the external camera image 68G on the diagnostic device 16A.

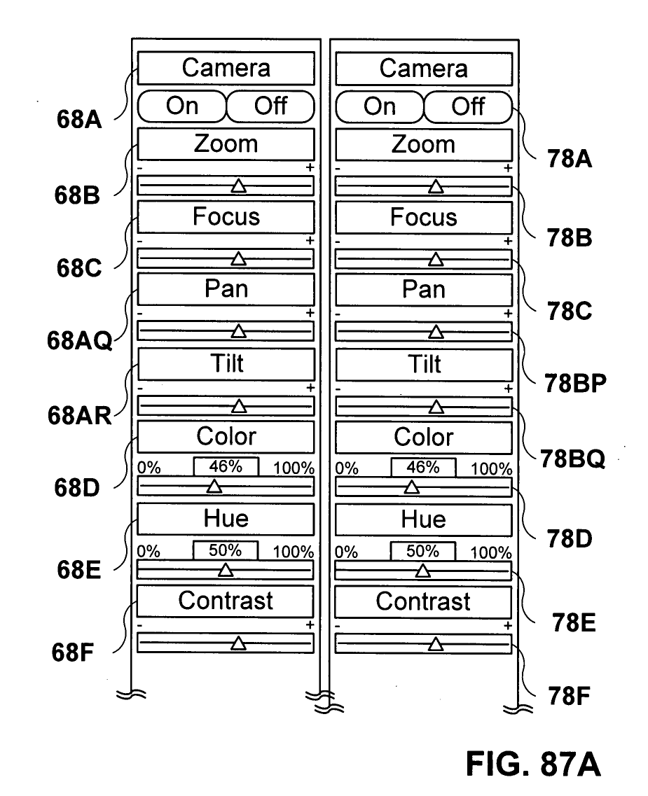

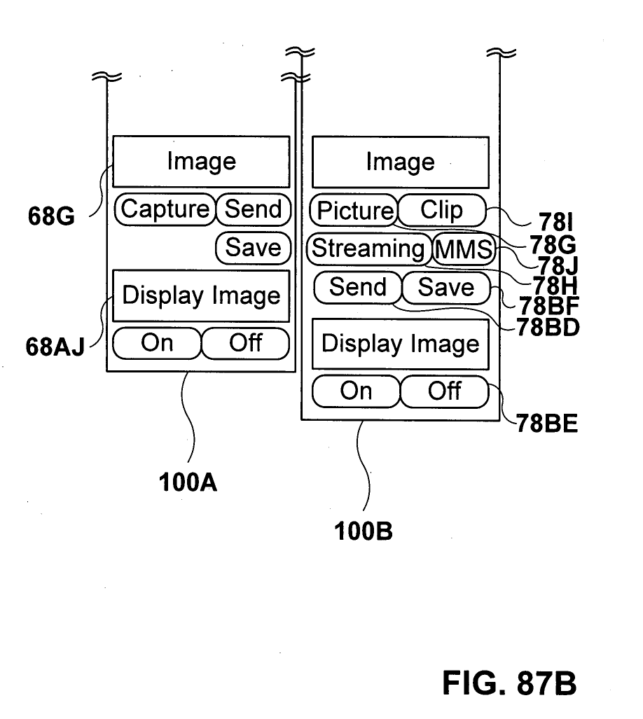

FIG. 87 shows an eleventh embodiment of a functional block diagram 100 of embedded software 18A that enables remote functional control of the data devices 80 for the diagnostic means 16A as well as a software application 26A that enables remote functional control of the data devices 80 connected to the diagnostic means 16A. The functions shown in 100A are deployed as a component of 18A, the software embedded in a diagnostic means 16A; those in 100B as a component of the diagnostic, display and control software application 26A.

The functions 68 embedded in a diagnostic device 16A for controlling the data device 80 the external camera 80K include, but are not limited to:
Turn the camera 80K on or off 68A;
Zoom 68B the camera 80K;
Focus 68C the camera 80K;
Pan 68AS camera 80K;
Tilt 68AT camera 80K;
Adjust the camera 80K color 68D;
Adjust the camera 80K hue 68E;
Adjust the camera 80K contrast 68F;
Turn illumination 48C on or off 68J;
Adjust the illumination 68K;
Capture an image 68G; and
Turn the camera 80K display 68AS on or off.

The functional control means 78 in the diagnostic, display and control software application 26A for remotely controlling the external camera 80K via a diagnostic device 16A include, but are not limited to:
An “off” button and an “on” button 78A for controlling 68A the camera 80H;
A slider 78B that adjusts the zoom 68B from zero percent (0%) to one hundred percent (84%);
A slider 78C that adjusts the camera focus 68C from minus (−) to plus (+);
A slider 78BP that adjusts the camera pan 68AQ from minus (−) to plus (+);
A slider 78BQ that adjusts the camera tilt 68AR from minus (−) to plus (+);
A slider 78D that adjusts the color 68D from zero percent (0%) to one hundred percent (84%);
A slider 78E that adjusts the hue 68E from zero percent (0%) to one hundred percent (84%);
A slider 78F that adjusts the contrast 68F from minus (−) to plus (+);
An “off” button and an “on” button 78M for controlling 68F illumination 48C;
A slider 78N that adjusts the illumination 48C from zero percent (0%) to one hundred percent (100%);
A button 78G for capturing 68G the image 44F as a picture 44F1;
A button 78H for capturing 68G the image 44F as streaming video 44F2;
A button 78I for capturing 68G the image 44F as a video clip 44FB3;
A button 78J for capturing 68G the image 44F as a multi-media message (MMS) 44F4;
A button 78BE for sending 68G the image 44F; and
An “off” button and an “on” button 78BF for controlling the display 68AJ of the camera 80H.

An additional functional control means 78BG allows the healthcare provider 20C to save the image 44F to the patient's 12A Electronic Medical Record (EMR) 60. The software application 26A automatically tags the image 44F with the date 56A and current time 56B.

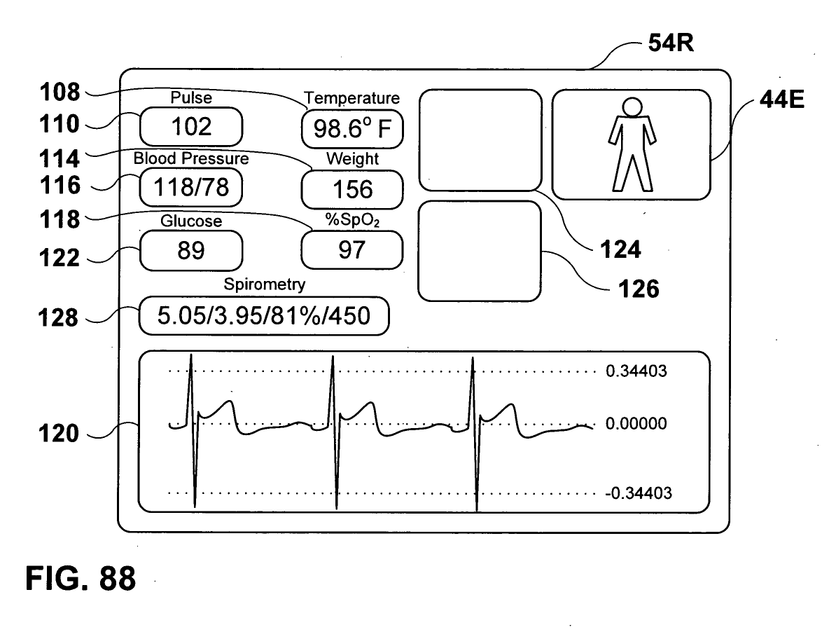

The external camera 80K image 44E appears on the healthcare provider's 20C diagnostic, display and control means 24A as shown in FIG. 88.

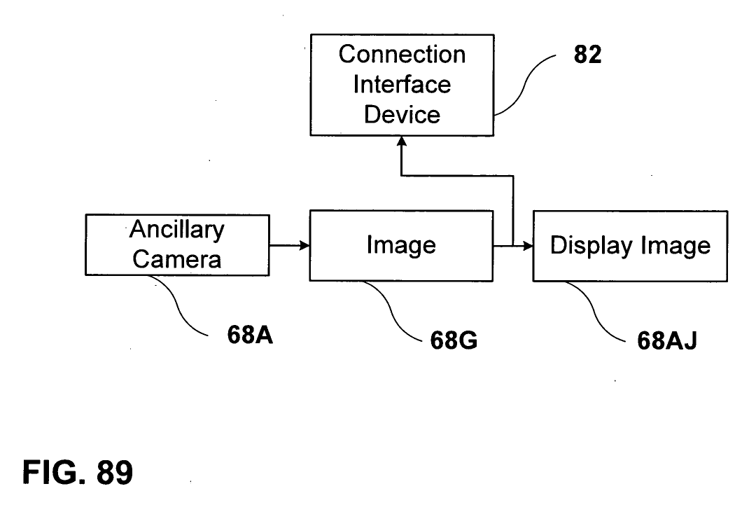

FIG. 89 shows the image 68G fed to the connection interface device 82 for connection 50 to a device or terminal 16.

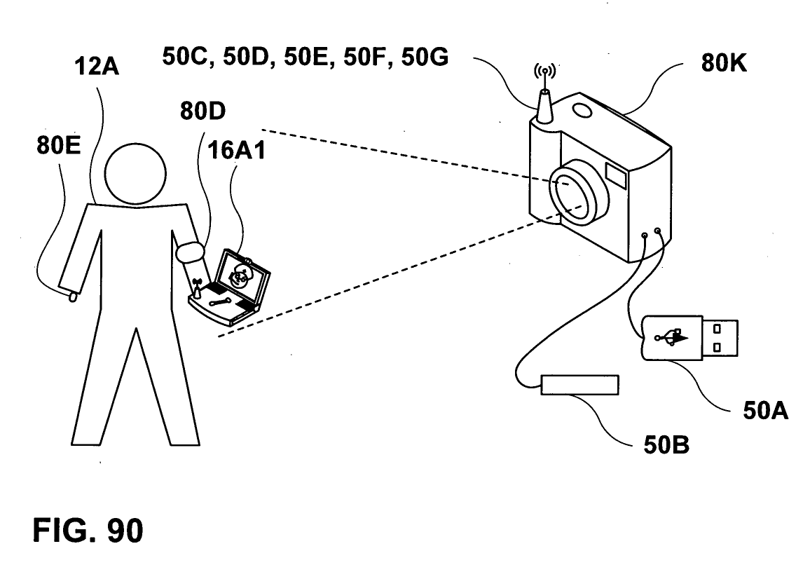

The external camera 80K is particularly useful to the healthcare provider 20C for observing the patient 12A as he or she utilizes the diagnostic means 16A1, including data devices 80, as shown in FIG. 90. In this embodiment the external camera 80K is set away from the patient 12A so that the healthcare provider 20C can see what the patient 12A is doing, especially in placing data devices 80 on his or her body.

### III. Alternative Embodiments of the Diagnostic Means

An embodiment of the diagnostic means 16A has thus far been described as a Virtual Visit Book™ (VVB) 16A1. A first alternative embodiment of the diagnostic means 16A is a Personal Computer (PC) equipped with a camera 42D. All PCs 16A2, 16A3 have at least one data port 130A, a “line out” port 130B for plugging in an external speaker 40C, and an audio port 130C for plugging in an external microphone 40D. The combination of the line out port 130B and the audio port 130C and the may be used for an external headset 40E. Sometimes the line out port 130B and the audio port 130C are combined in a single jack.

Data devices 80 may be connected 50 to a PC 16A2, 16A3 using wires or wirelessly. Today, all PCs 16A2, 16A3 have at least one USB port 50A and at least one FireWire port 50B. Modern day laptops 16A3 have Bluetooth 50C and WiFi 50D capabilities built in. It is expected that in the near future they may also have UWB 50E, ZigBee 50F and WiMax 50G built in.

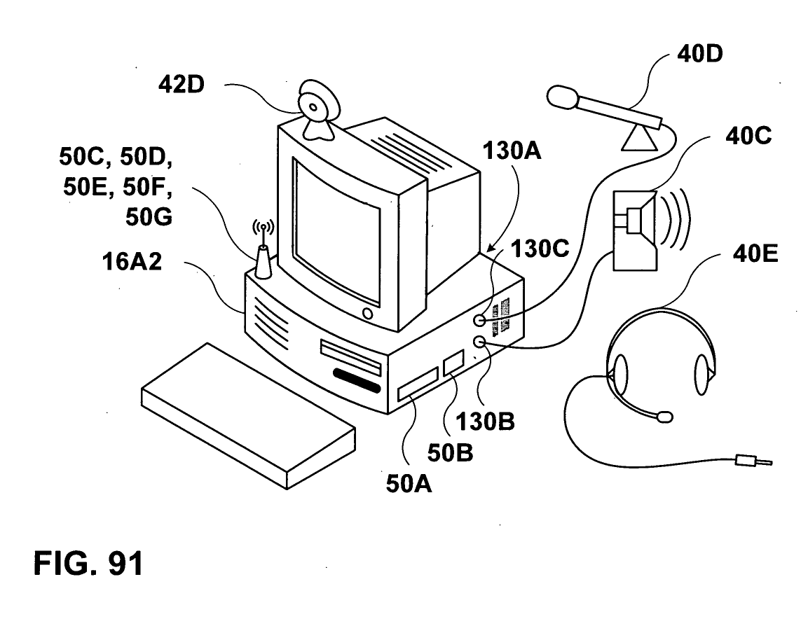

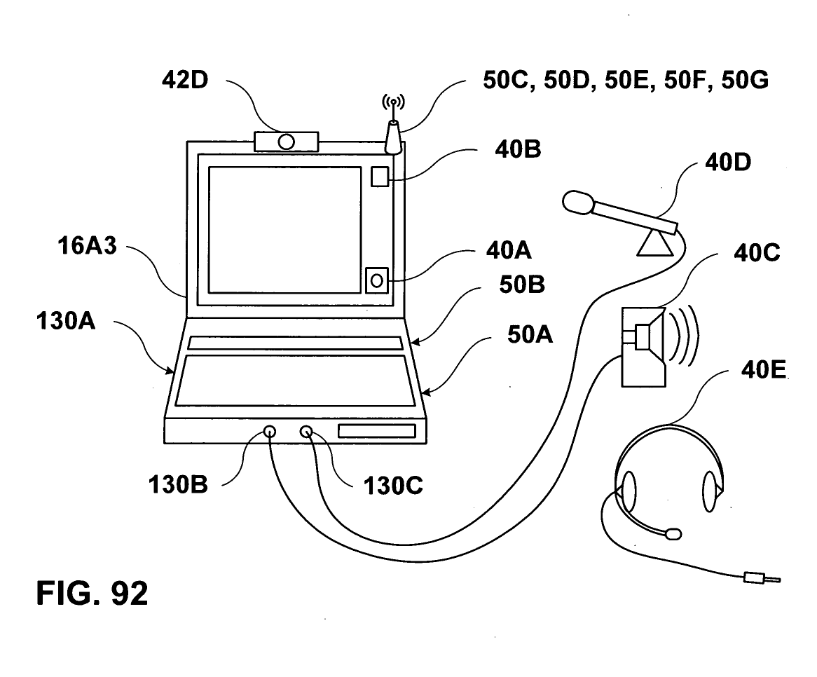

FIG. 91 shows a desktop PC 16A2 with an attached camera 42D, and FIG. 92 shows a laptop PC 16A3 with a built in camera 42. Most laptop PCs 16A3 have built in speakers 40A and many have built in microphones 40B.

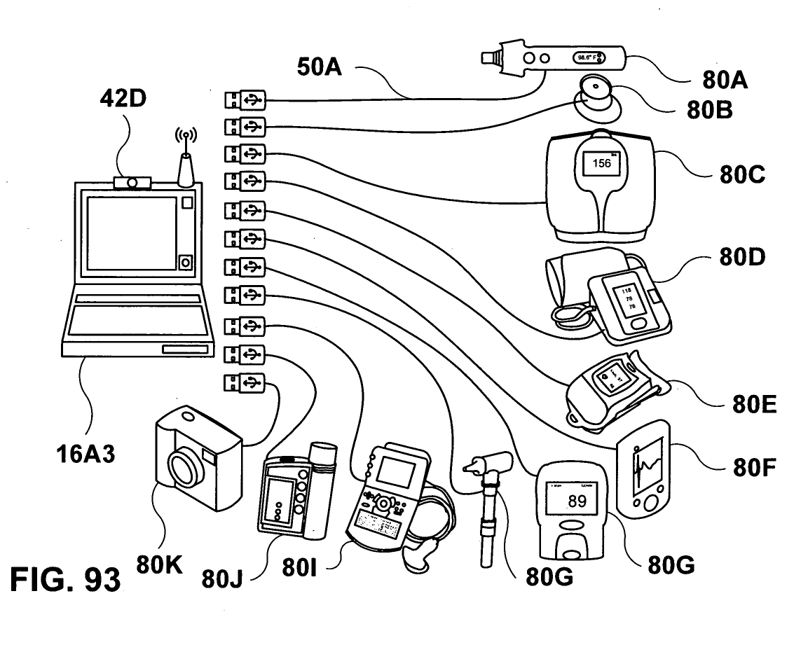

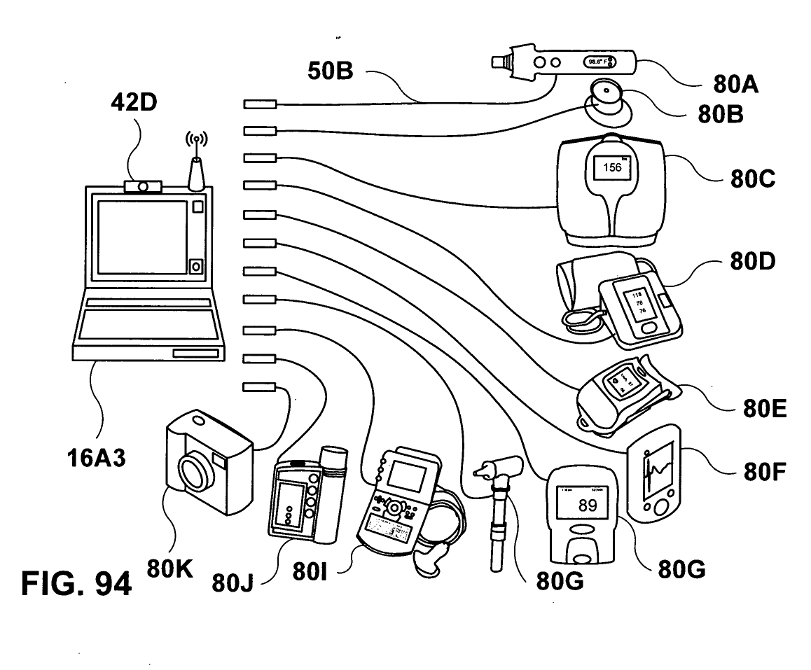

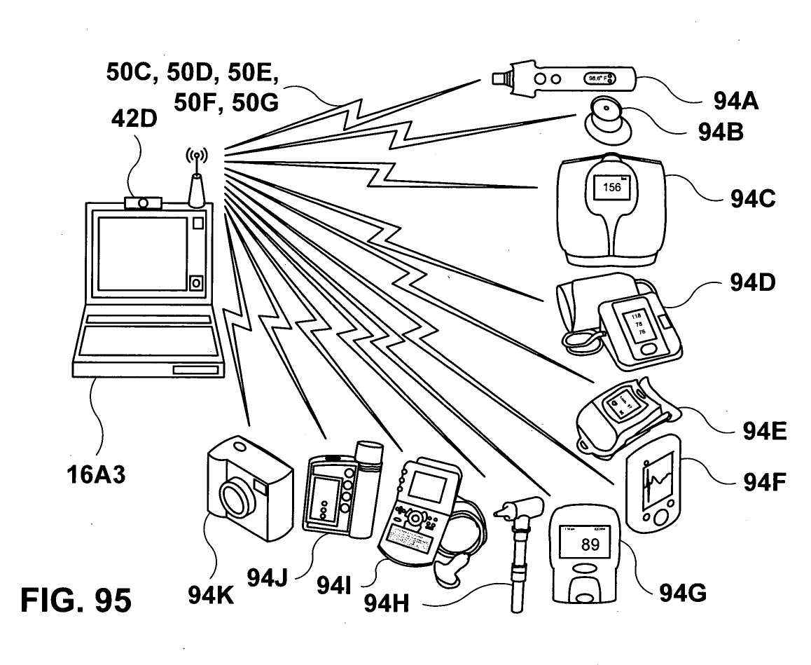

All of the data devices 80 may be connected to a desktop PC 16A2 or a laptop PC 16A3 via a wired connection as shown in FIG. 93 for USB 50A and in FIG. 94 for FireWire 50B, or wireless connection as shown in FIG. 95 via Bluetooth 50C, WiFi 50D, UWB 50E, ZigBee 50F or WiMax 50G.

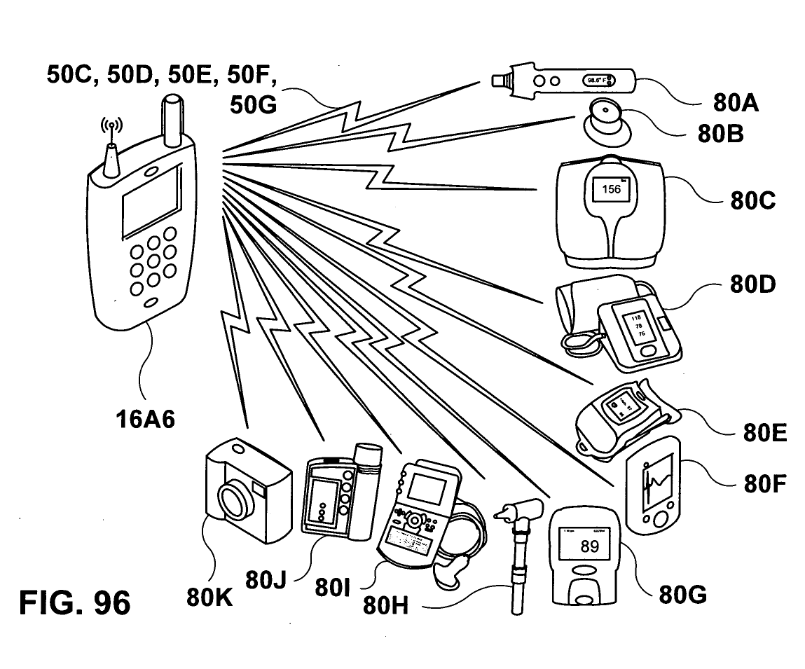

A further alternative embodiment of a diagnostic means 16A is a cellular or Personal Communications Service (PCS) wireless phone 16A6, also termed a “cell phone.” Many cellular and PCS wireless phones 16A6 are today enabled with Bluetooth 50C. Cellular and PCS wireless phones 16A6 enabled with WiFi 50D are just becoming available from Avaya®, E-TEN®, Hewlett-Packard (HP)®, Microsoft®, Motorola®, NEC®, Proxim® and others. Data devices 80 may be connected to these new WiFi-enabled cellular and PCS wireless phones 16A6 as shown in FIG. 96. In the future manufacturers may add UWB 50E, ZigBee 50F and WiMax 50G chip sets to cellular and PCS wireless phones 16A6.

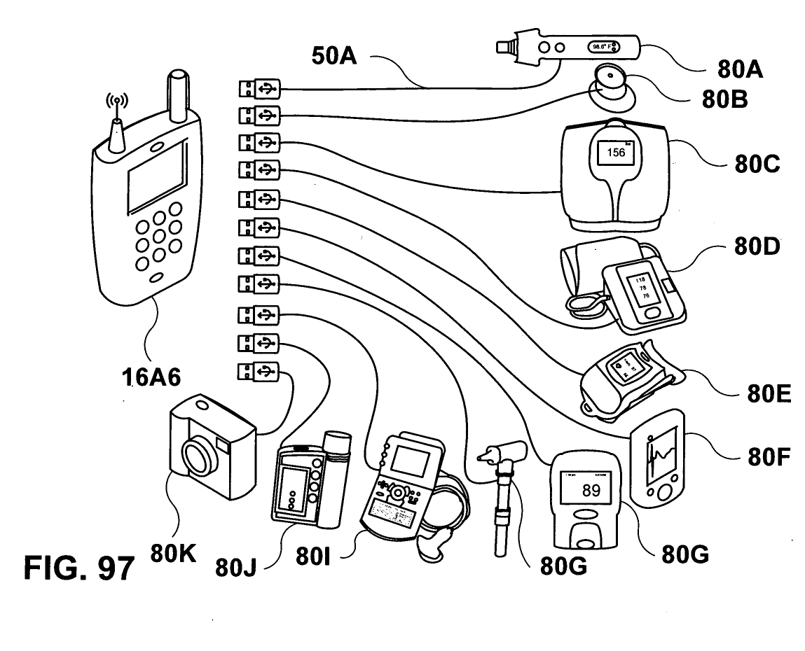

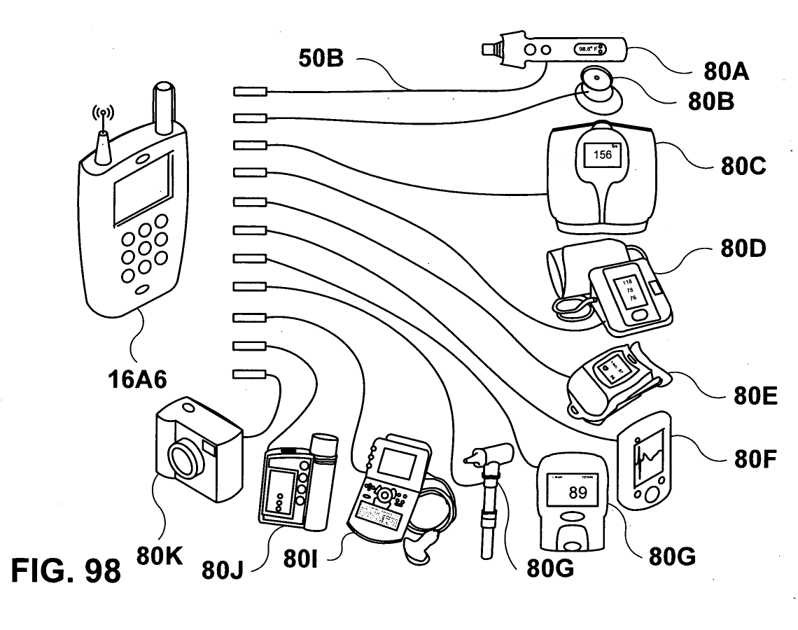

Most cellular and PCS wireless phones 16B have a data port 130A. Today, most of these data ports 130A are proprietary. In the future there is no reason for cellular and PCS wireless phones 16A6 not having USB 50A and/or FireWire 50B ports. FIG. 97 shows connection 50 of data devices 80 to a cellular or PCS wireless phone 16A6 via USB 50A; FIG. 98 shows connection 50 of data devices 80 to a cellular or PCS wireless phone 16A6 via FireWire 50B.

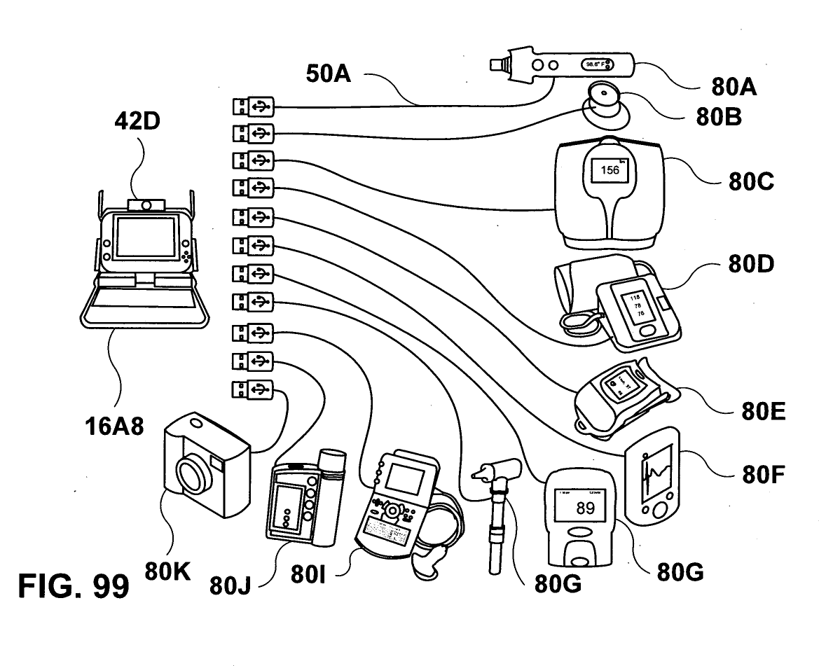

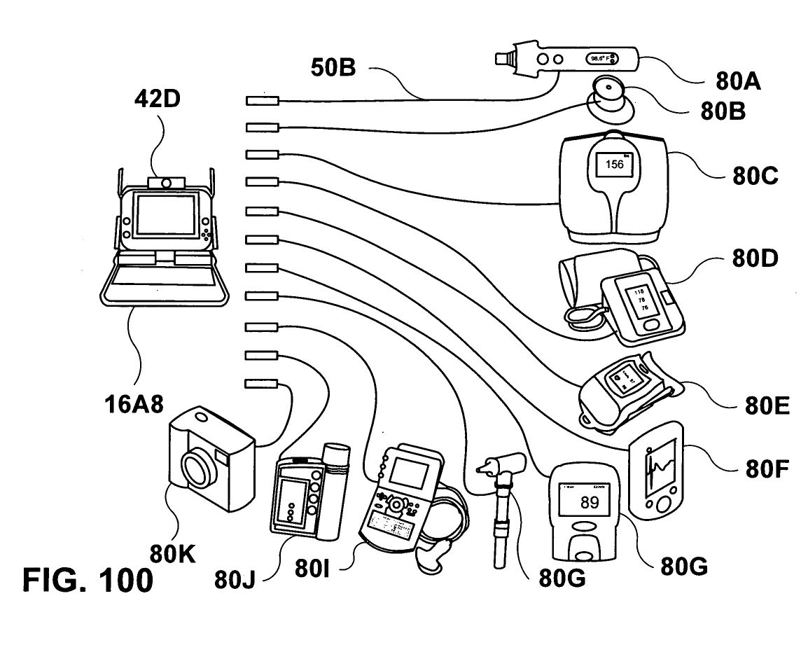

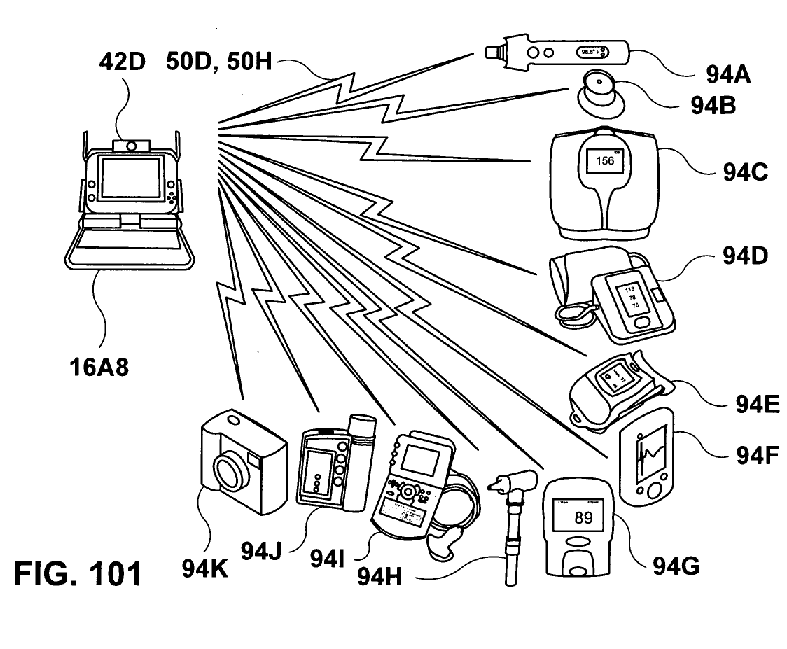

A further alternative embodiment of a diagnostic means 16A is the One Laptop Per Child (OLPC) XO laptop 16A8. Data devices 80 may be connected 50 to an OLPC 16A8 using wires or wirelessly. FIG. 99 shows connection 50 of data devices 80 to an OLPC 16A8 via USB 50A; FIG. 100 shows connection 50 of data devices 80 to an OLPC 16A8 via FireWire 50B; FIG. 101 shows connection 50 of data devices 80 to an OLPC 16A8 wirelessly via WiFI 50D and via a wireless mesh network 50H.

There are other developing and emerging wireless waveforms and network topologies that may be used in the Personal Healthcare Assistant 10.

Some medical conditions require continuous or semi-continuous monitoring. In one embodiment a diagnostic means 16A can be left on and connected to a healthcare provider 20C and the image 44 or data 86 from data devices 80 continuously transmitted to the healthcare provider 20C. Alternatively and more practically, the patient 12A can save images 44 or data 86 from data devices 80 in the diagnostic means 16A for transmission to a healthcare provider 20C on a scheduled or an ad hoc basis.

### IV. Alternative Applications of the Personal Healthcare Assistant

The Personal Healthcare Assistant 10 has numbers of applications beyond remote triage, diagnosis and healthcare monitoring. A first alternative embodiment addresses remote triage and monitoring of elderly patients 12A, particularly those in assisted living environments. Elderly patients 12A in assisted living environments are often provided a lanyard-based or clothing clipped button device that the patient or potential patient 12A can push to alert the staff in the event of an emergency. Pushing the button usually turns on a light in a monitoring station and causes an attendant to go to the patient's 12A unit to assess the situation. More advanced versions of the “button” include a microphone that enables the patient 12A to talk to the monitoring attendant.

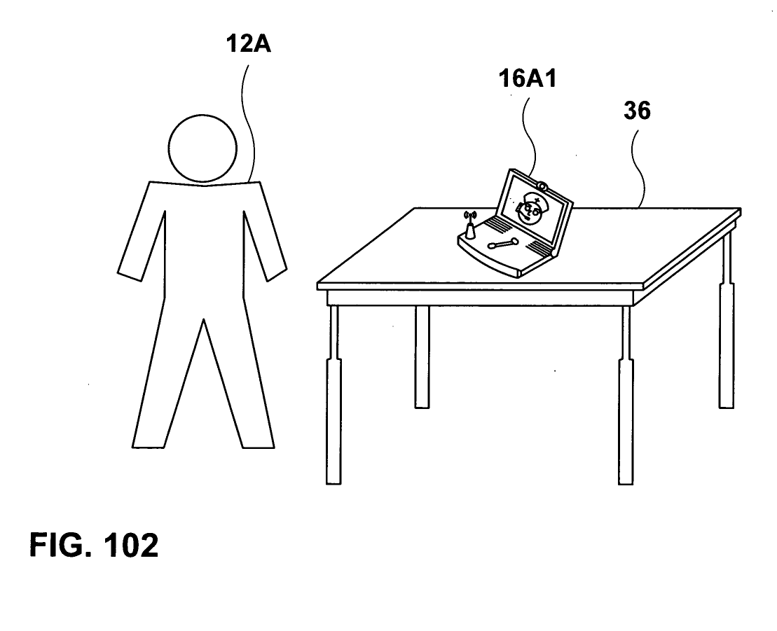

An embodiment of the Personal Healthcare Assistant 10A to be deployed in an assisted living environment is shown in FIG. 102. In this embodiment a patient 12A has the diagnostic device 16A, specifically a Virtual Visit Book™ (VVB) 16A1, on a table 36. If the elderly patient 12A has a problem, he or she simply opens the VVB 16A1 and is immediately visually connected to a healthcare provider 20C.

In an alternative embodiment the VVB 16A1 could be programmed to connect to other persons and/or locations such as a monitoring station or “911.”

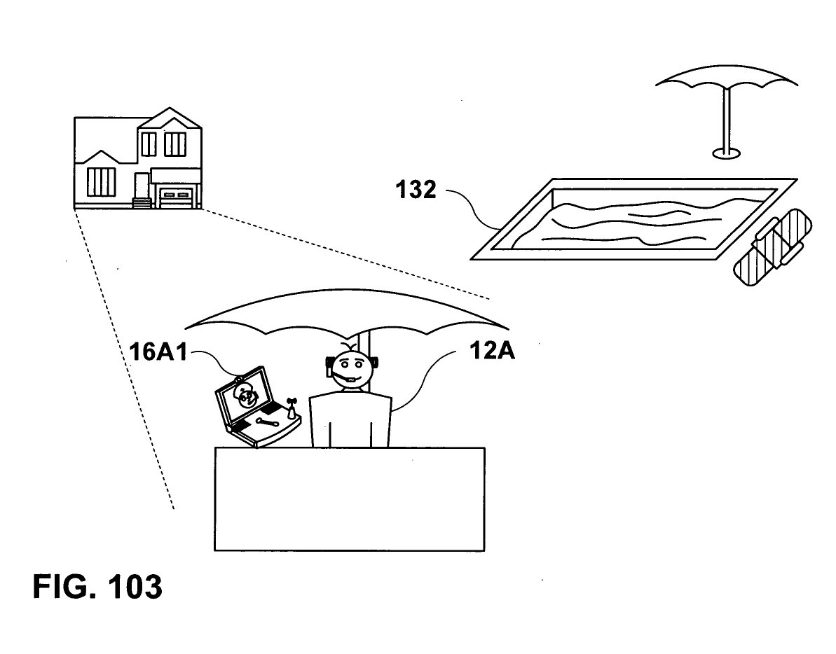

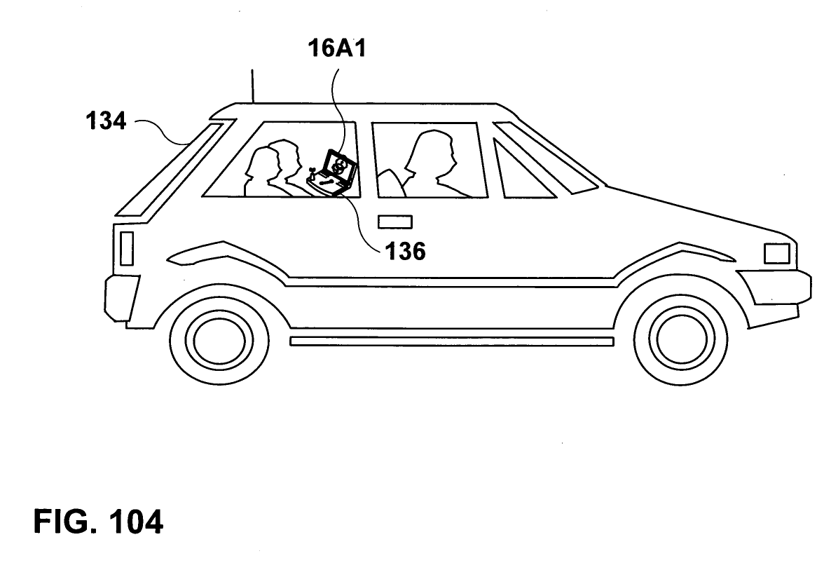

In a preferred embodiment the VVB 16A1 is battery powered and, consequently, portable. Therefore, a patient 12A may be able to contact a health service provider 20C from other than a fixed location. FIG. 103 shows a patient 12A sitting next to a pool 132 with a VVB 16A1. A VVB 16A1 may even be used on the move, for example, in an automobile as is shown in FIG. 104.

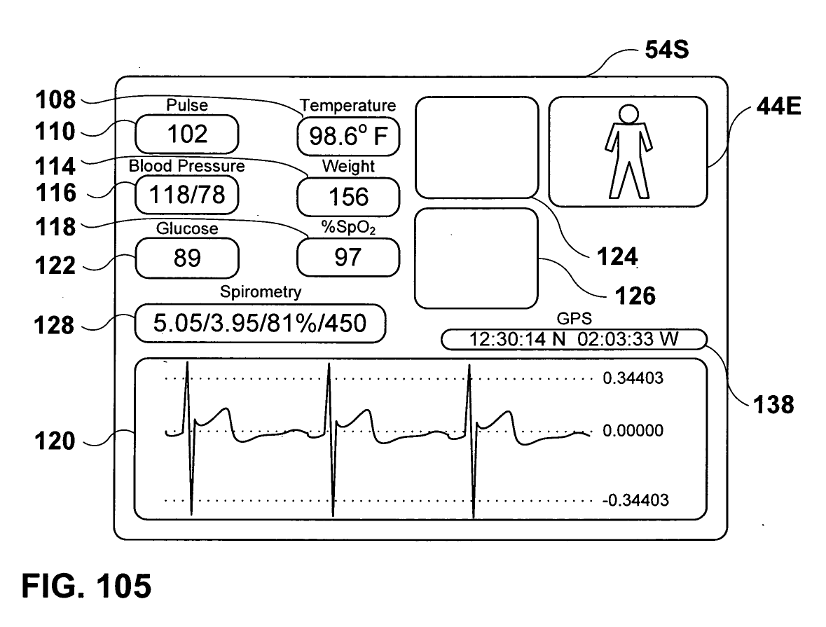

In the instant embodiment it is beneficial to embed a Global Positioning System (GPS) receiver 136 into the VVB 16A1 as shown in FIG. 104 so that the healthcare provider 20C can know the location of the patient 12. The diagnostic, display and control software application 26A deployed on the diagnostic, display and control means 24A has the ability to receive and display the GPS 136 data as shown in FIG. 105. The GPS 136 location appears in window 138 on the healthcare provider's 20C diagnostic, display and control means 24A as shown in FIG. 105. In a preferred embodiment the display of the GPS 136 data is in the form of a map.

A diagnostic device 16A with embedded GPS 136 is also useful for keeping track of patients 12A with Alzheimer's or other dementia disablements.

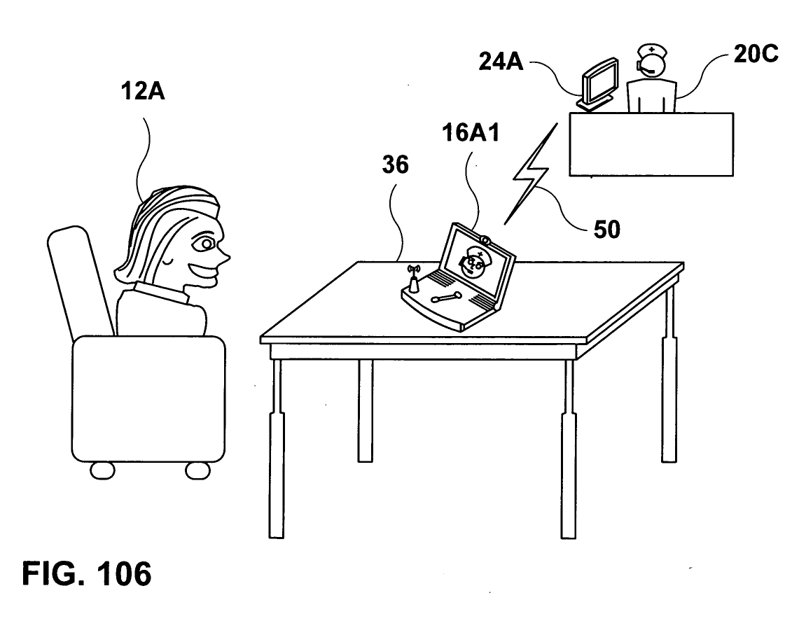

The follow-up care for post traumatic stress syndrome patients involves weekly, or more frequent, interaction between the patient 12A and the healthcare provider 20C. Today, this interaction requires a patient 12A to travel to a brick-and-mortar facility. Post traumatic stress syndrome counseling session involve questioning the patient 12A about his or her emotional state as well as the status of his or her relationships with others, in the home, in the workplace and otherwise. Post traumatic stress syndrome patients 12A are often reluctant to be completely forthcoming to the Healthcare provider 20C out of embarrassment or otherwise. In face-to-face interactions the healthcare provider can assess the veracity of the patient's 12A answers to questions by observing the patient's face, particularly their eyes, and their demeanor. A post traumatic stress syndrome patient 12A having a VVB 16A1 facilities on-demand real-time counseling interactions while allowing the healthcare provider 20C to assess a patient's 12A condition with having the patient 12A directly in front of them. See FIG. 106.

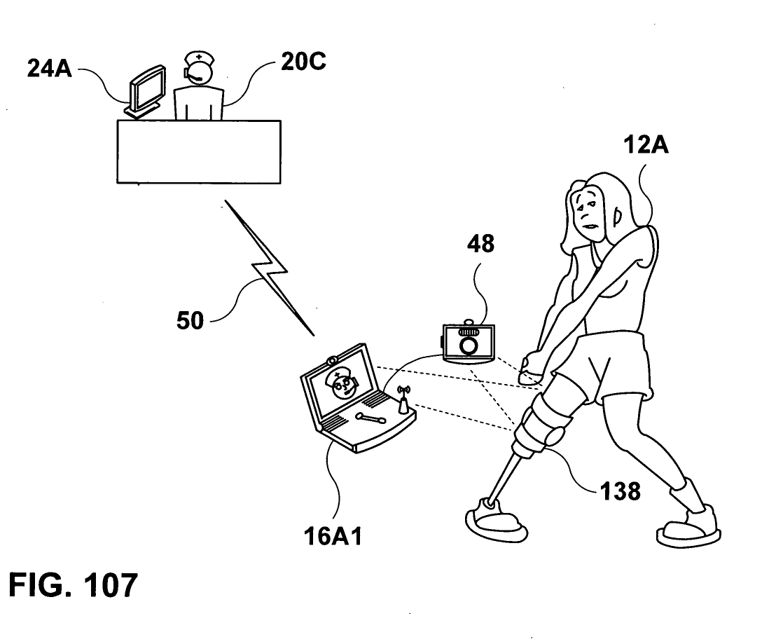

Another follow-up care application is the fit of prosthetic devices. Prosthetic devices are fit by medical technicians in specialized clinics. Over time the human body adapts to the fit of the prosthetic device, which may result in stress or discomfort to the patient 12A. Today the patient 12A has to travel back to a prosthetic clinic to have the prosthesis adjusted. Real-time adjustment of the prosthesis 138 is possible using a VVB 16A1 independently or in conjunction with a Visual Electronic Medical Record (VEMR™) device 48. See FIG. 107.

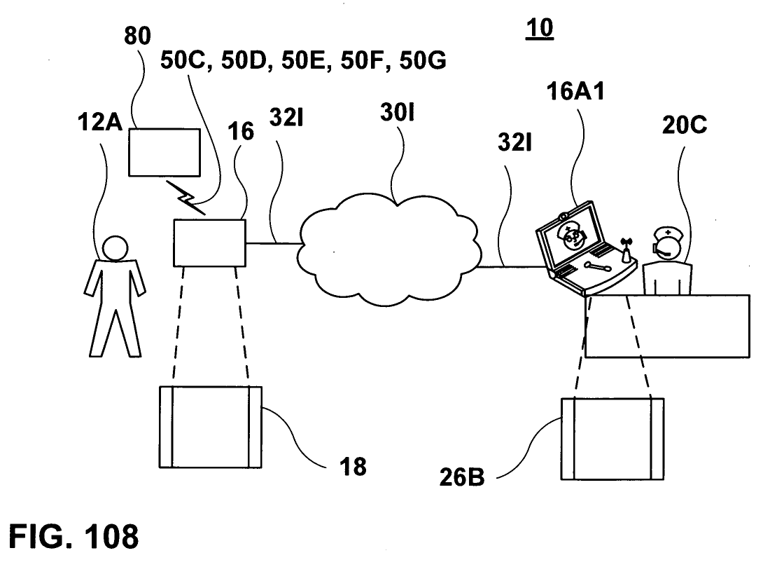

All of the embodiments described thus far have the Healthcare provider 20C in a fixed location. The technologies of the Personal Healthcare Assistant 10 enable the healthcare provider 20C to work from home or on the move. In the embodiment shown in FIG. 108 the healthcare provider 20C is at home with a VVB 16A1 as the diagnostic, display and control means 24B, and connected 32I to the Internet 301.

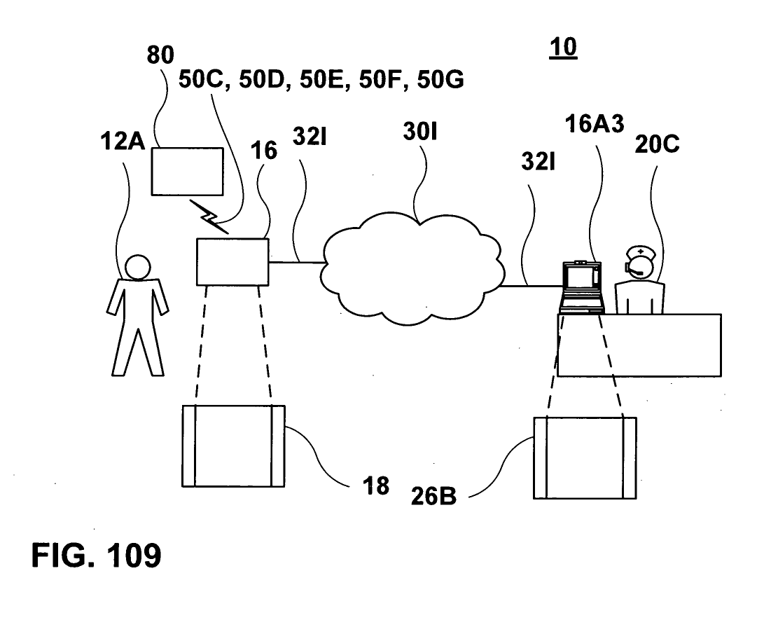

In the embodiment shown in FIG. 109 the healthcare provider 20C is at home with a laptop PC 16A3 as the diagnostic, display and control means 24B, and connected 32I to the Internet 30I. The diagnostic, display and control software application 26B is Internet-enabled.

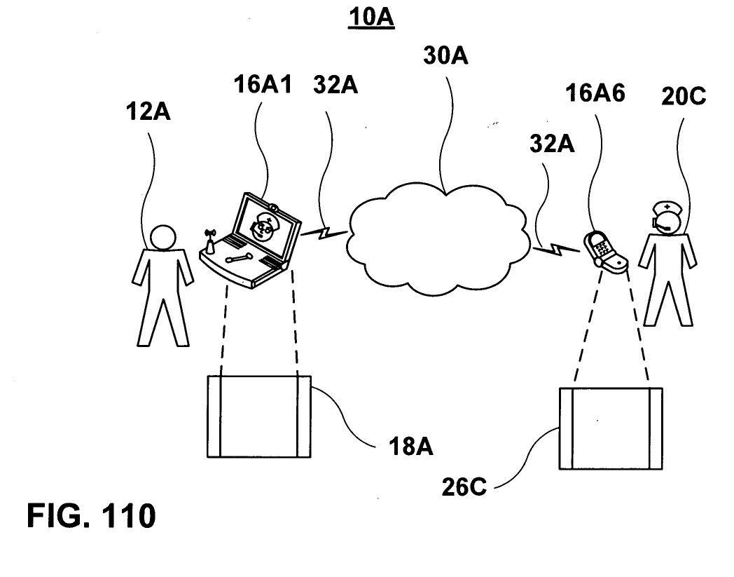

In the embodiment shown in FIG. 110 the healthcare provider 20C can be on the move. In this embodiment the diagnostic, display and control means 24C is a cellular or PCS wireless phone 16A6 with the diagnostic, display and control software application 26C embedded into it.

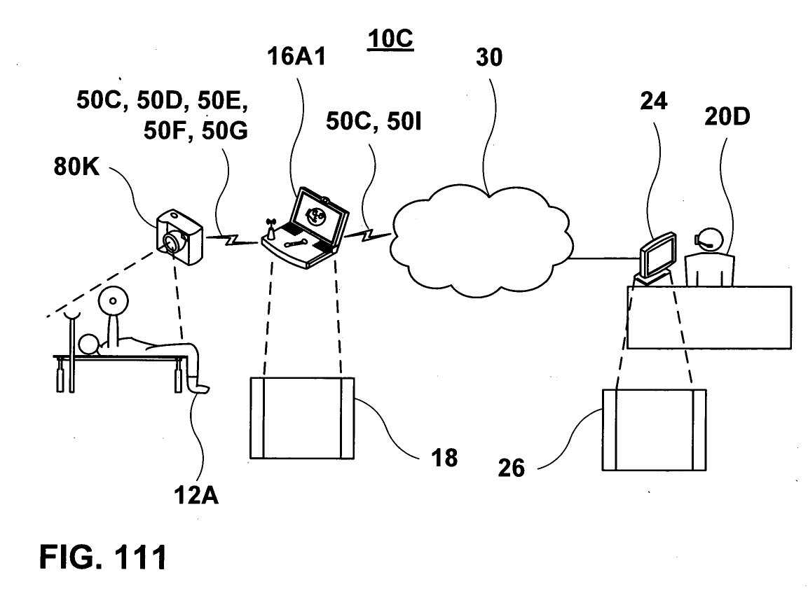

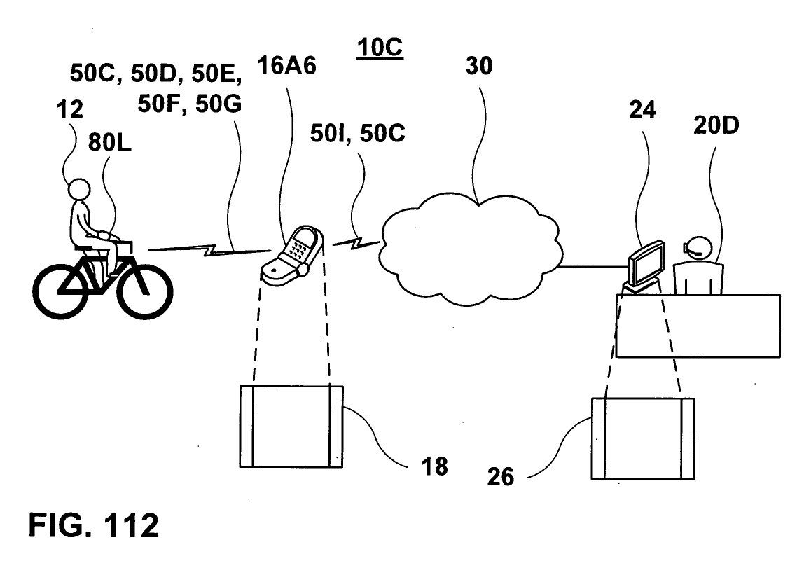

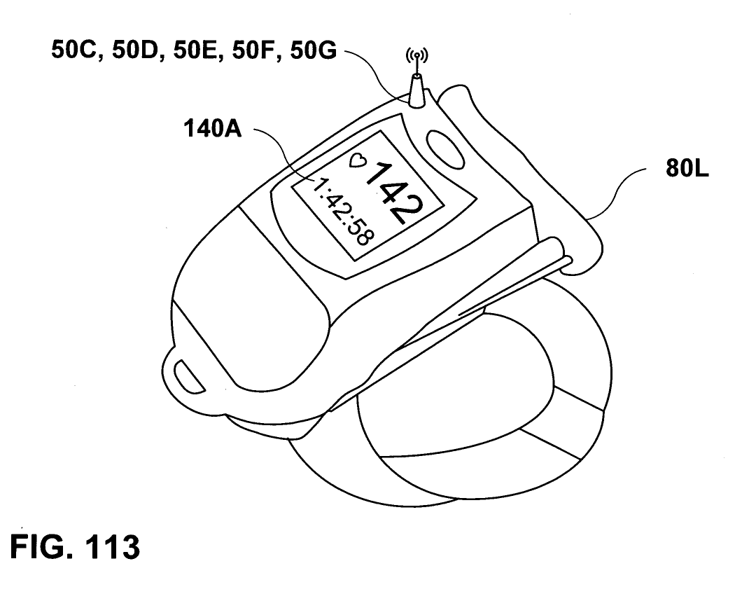

The Personal Healthcare Assistant 10 may also be used for physical rehabilitation and athletic performance coaching. In this embodiment of the Personal Healthcare Assistant 10C the healthcare provider 20C is replaced by a physical therapist or athletic coach 20D. A preferred embodiment of this application is shown in FIG. 111. An external camera 80K is deployed so the physical therapist or athletic coach 20D can observe the patient or potential patient 12A lifting weights. By turning on the speaker phone 40 the physical therapist or athletic coach 20D can correct the patient's or potential patient's 12A body position by speaking to him or her. The image from the external camera 80K can be transmitted to the VVB 16A1 via Bluetooth 50C, WiFi 50D, UWB 50E, ZigBee 50F or WiMax 50G. Similarly, the VVB 16A1 may communicate with the network 30 using cellular or PCS frequencies 501 or WiFi 50D, if enabled. Although FIG. 112 shows the coaching in a fixed setting, there is no reason why the coaching cannot take place while moving, for example, on a bicycle. In this embodiment, shown in FIG. 113 the patient 12A wears a heart rate monitor 80L that communicates with to the cellular or PCS wireless phone 16A6 via Bluetooth 50C, WiFi 50D, UWB 50E, ZigBee 50F or WiMax 50G as shown in FIG. 112. Heart rate monitors 80L are commercially available from Acumen®, Cardiosport®, Mio®, Polar®, Reebok® and others, and often include watches 140 stop watches 140A

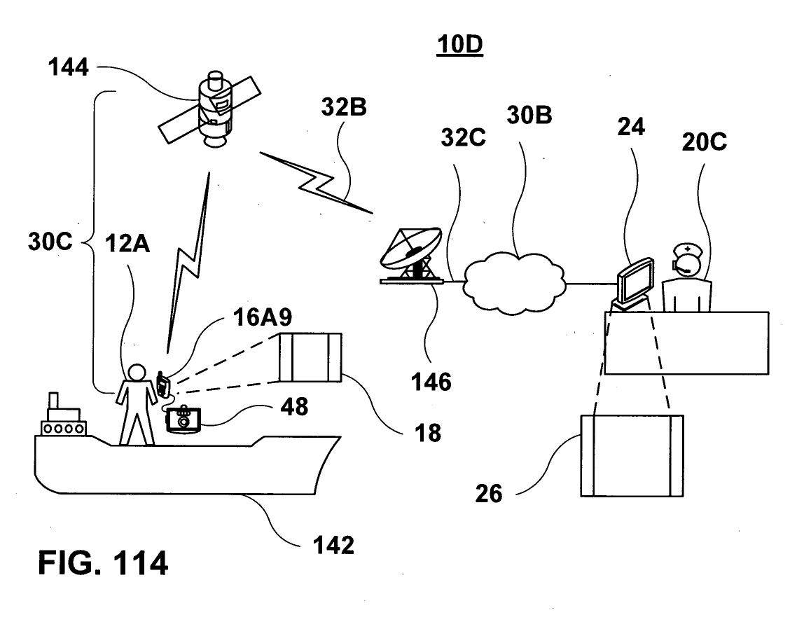

An additional embodiment of the Personal Healthcare Assistant 10D is shown in FIG. 114. In this embodiment a patient 12A in a remote location, in this embodiment aboard a ship 142, uses a satellite phone 16A9 as a diagnostic means 16A as well as a VEMR™ 48. The satellite phone 16A9 connects 32B to a satellite 144 and then to an earth station 146 connected 32C to the PSTN 30B. Satellite network 32B capacity is available from Iridium®, Globalstar®, Inmarsat®, New Skies®, Intelsat® and others.

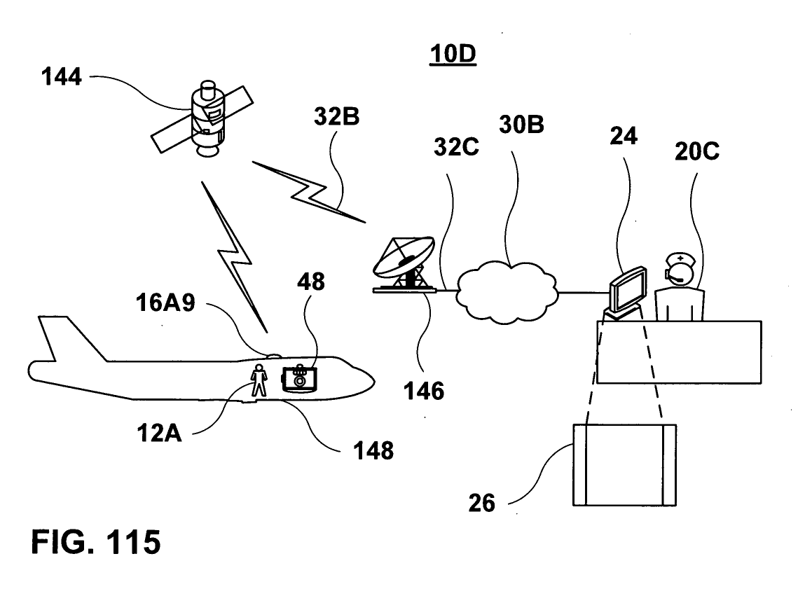

An additional embodiment of the Personal Healthcare Assistant 10D is shown in FIG. 115. In this embodiment a patient 12A aboard an airplane 148, uses a VEMR™ 48 and an aircraft satellite phone 16A10 to connect 32B to a satellite 144 and then to an earth station 146 connected 32C to the PSTN 30B. Aircraft satellite phone services are available from Inmarsat®, New Skies® and Intelsat®.

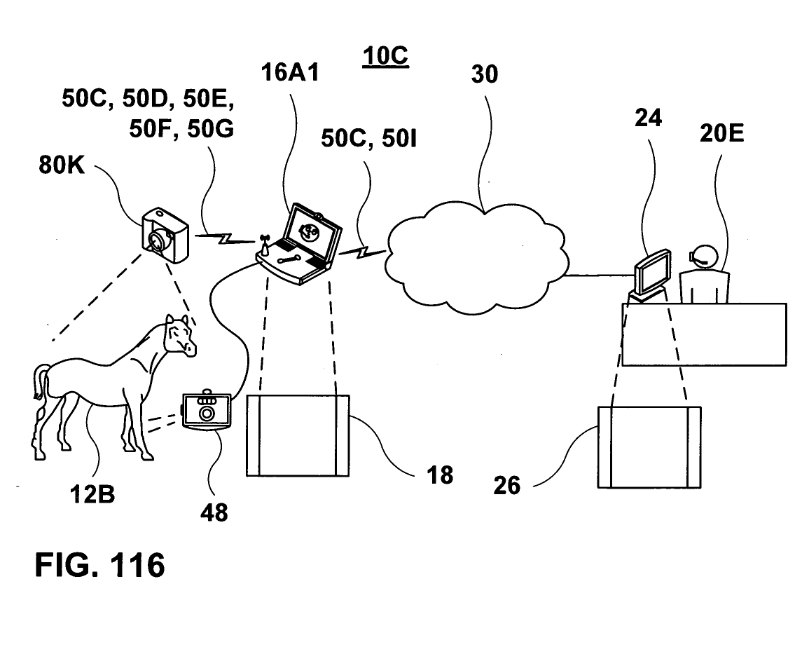

Although described thus far in human terms, the Personal Healthcare Assistant 10 may also be used to treat animals. In the embodiment shown in FIG. 116 the “patient” is an animal 12B, here a horse, and the healthcare provider is a veterinarian 20E.

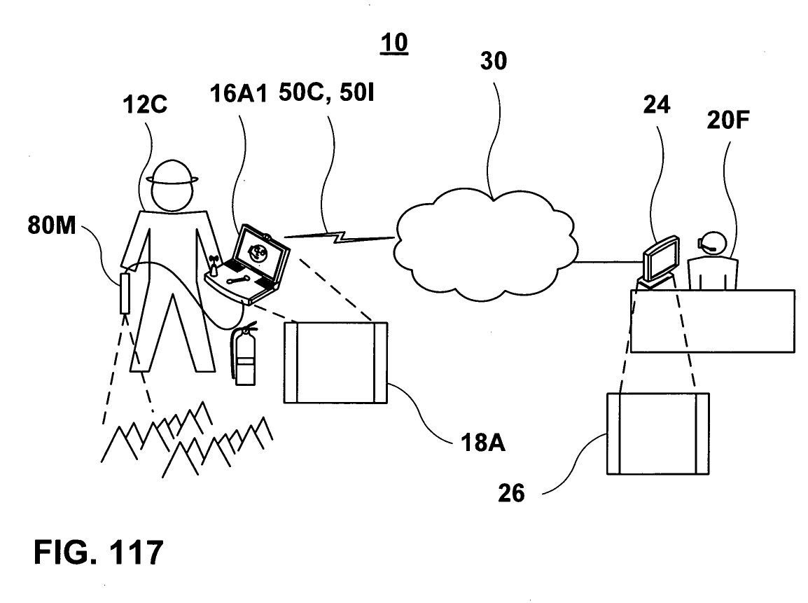

Although described thus far in healthcare terms, embodiments of the Personal Healthcare Assistant 10 may be used for consultations between field personnel and others. For example, in the embodiment shown in FIG. 117 a fire investigator 12C is using a chemical sniffer 80M attached to a VVB 16A1 to get data about the potential causes of a fire. The data is transmitted to a laboratory technician 20F for assessment.

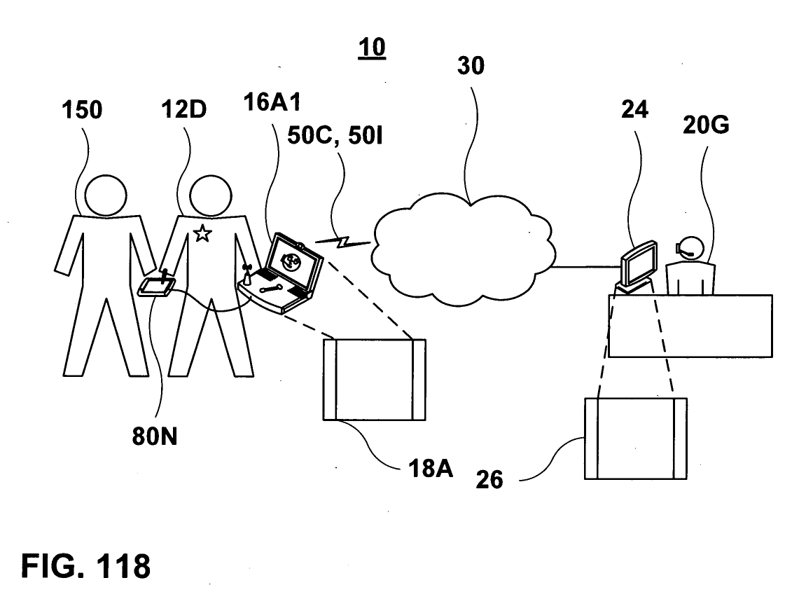

A further embodiment of the Personal Healthcare Assistant 10 is shown in FIG. 118 in which a policeman 12D uses a remote fingerprint device 80N attached to a VVB 16A1 to fingerprint a suspect 150. The fingerprint is transmitted to an analyst 20G for review and matching to fingerprint databases.

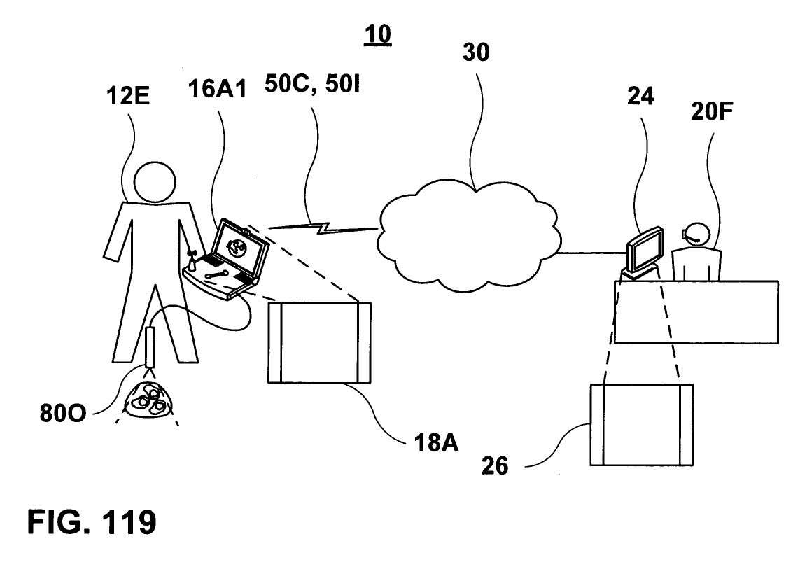

A further embodiment of the Personal Healthcare Assistant 10 is shown in FIG. 119 in which an engineer 12E uses a soil sampling device 80O attached to a VVB16A1. Characteristics of soil sample are transmitted to an laboratory technician 20F for assessment.

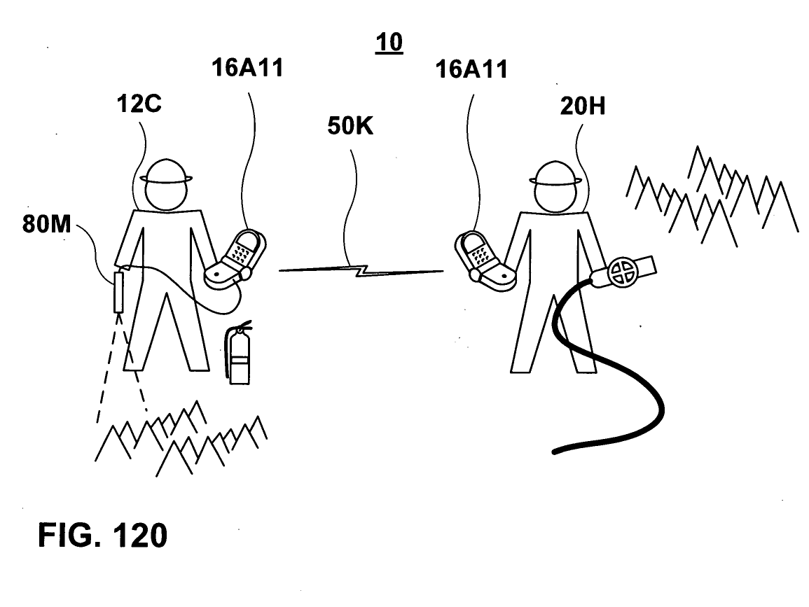

It is highly desirable in certain situations for remotely deployed personnel to share data in real time. FIG. 120 shows an embodiment of the Personal Healthcare Assistant 10 in which a fire investigator 12C is using a chemical sniffer 80M attached to a walkie-talkie 16A11 and directly transmits 50K that data in real time to a fire fighter 20H actively fighting the fire nearby.

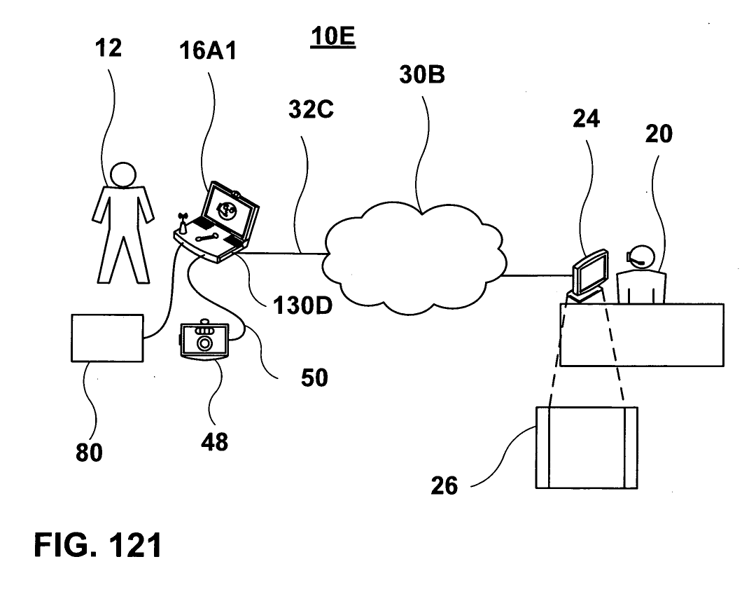

An alternative embodiment of the Personal Healthcare Assistant 10 is shown in FIG. 121. In this embodiment a VEMR™ 48 and data devices 80 are connected to a VVB 16A1 that includes a PSTN dial-up modem 130D that connects 32C to the PSTN 30B.

### V. Visual Electronic Medical Record (VEMR™) Attributes

When a healthcare provider 20C examines and assesses a patient 12A today, the healthcare provider 20C enters information into the patient's Electronic Medical Record (EMR) 60, often via an in-room terminal 64. If the healthcare provider 20C observes a wound 52 or other attributes of the patient's 12A condition or demeanor, the healthcare provider 20C typically must describe the wound, condition or demeanor in words. Such entry takes time and it is often difficult to describe such wound, condition or demeanor with appropriate detail. Being able to take a picture or capture video and put that in the patient's EMR 60 saves time and increases the detail and accuracy of the observations. The Visual Electronic Medical Record (VEMR™) device 48 is designed to save the healthcare provider 20C, increase the accuracy of patient 12A observation, and provide a basis for time series assessment of patient 12A condition. See FIGS. 16 through 18.

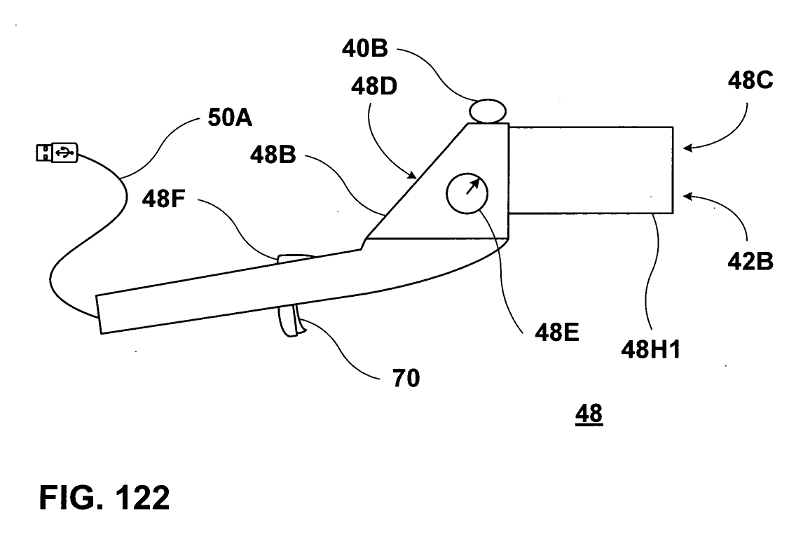

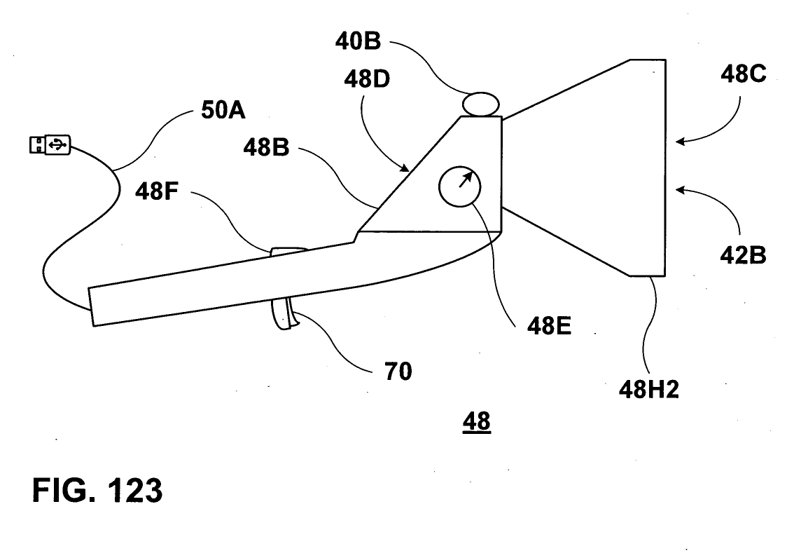

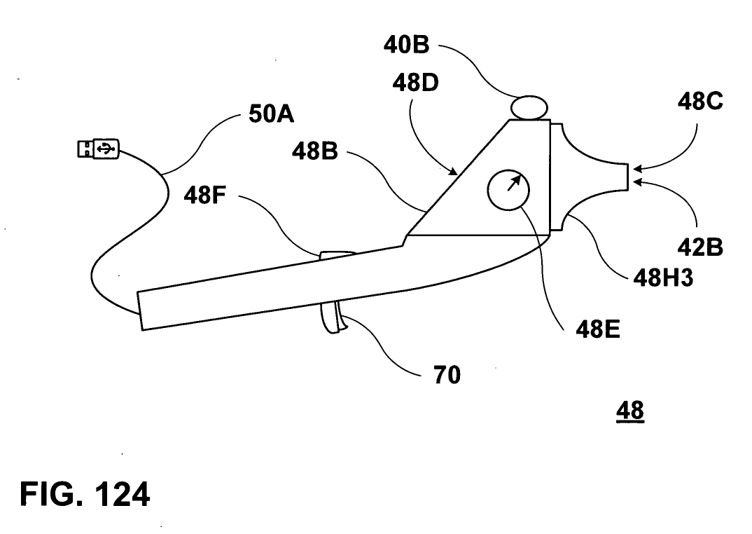

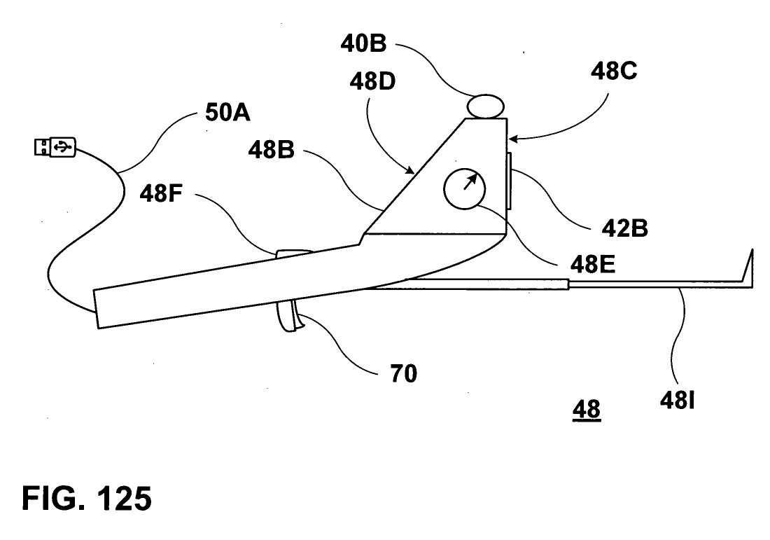

In a wound injury or dermatological patient 12A examination it is often desirable to track the healing process as shown in FIG. 21. Effectuating such tracking often relies upon measurement of the wound 52 or dermatological condition 52A. An embodiment of the VEMR™ device 48 includes a fixed focus “barrel” or “hood” 48H that may be placed over a wound 52 or dermatological condition 52A. FIG. 122 shows a barrel 48H1 for smaller wounds 52 or dermatological condition 52A. FIG. 123 shows a hood 48H2 for larger wounds 52 or dermatological conditions 52A. In some instances it may be desirable to fit the VEMR™ device 48 with a cone 48H3 similar to that used on an otoscope 80H, as shown in FIG. 124. For even larger wounds 52 or dermatological conditions 52A, an adjustable distance scale 48I may be attached to the VEMR™ device 48 as shown in FIG. 125.

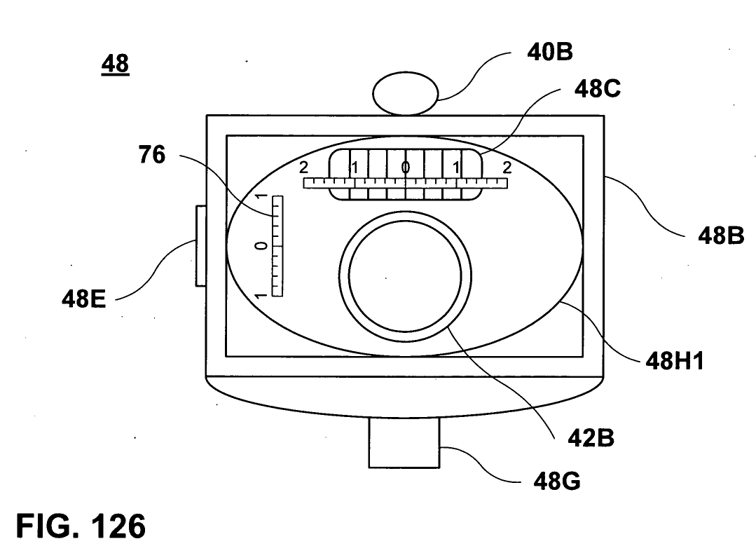

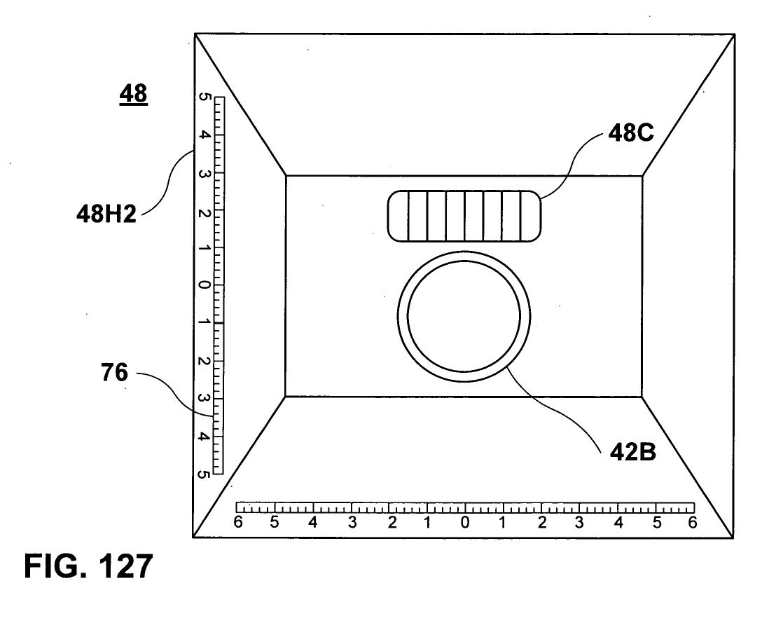

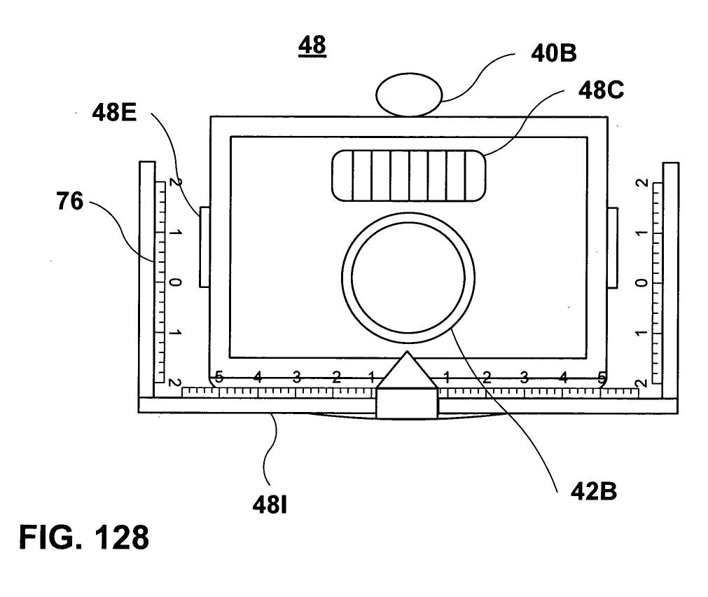

It is often desirable to have “x” and “y” dimensions of a wound 52 or dermatological condition 52A. An embodiment of such a ruler 76 in a barrel 48H1 is shown in FIG. 126. The rule 76 is transparent. An embodiment of a ruler 76 in a hood 48H2 is shown in FIG. 127. In this embodiment the rule 76 is attached to the edges of the hood 48H2. An embodiment of a ruler 76 attached to an adjustable distance scale 48I is shown in FIG. 128.

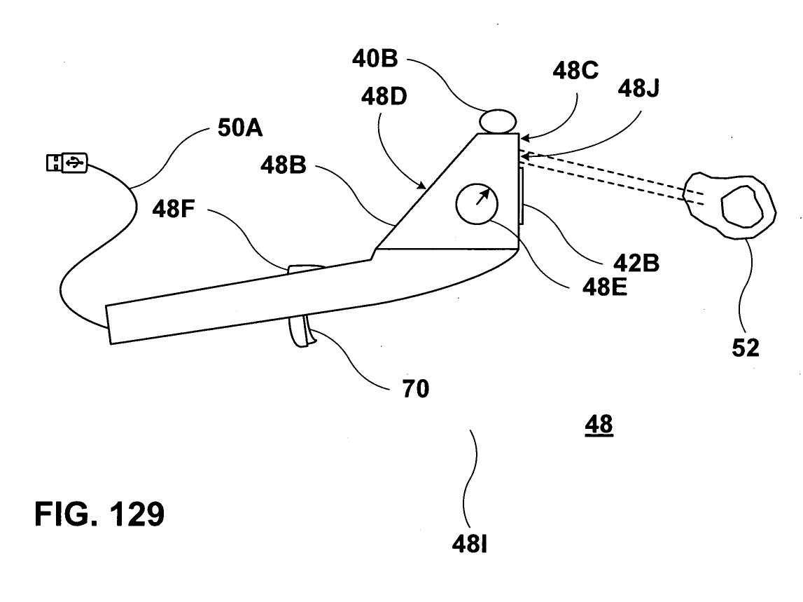

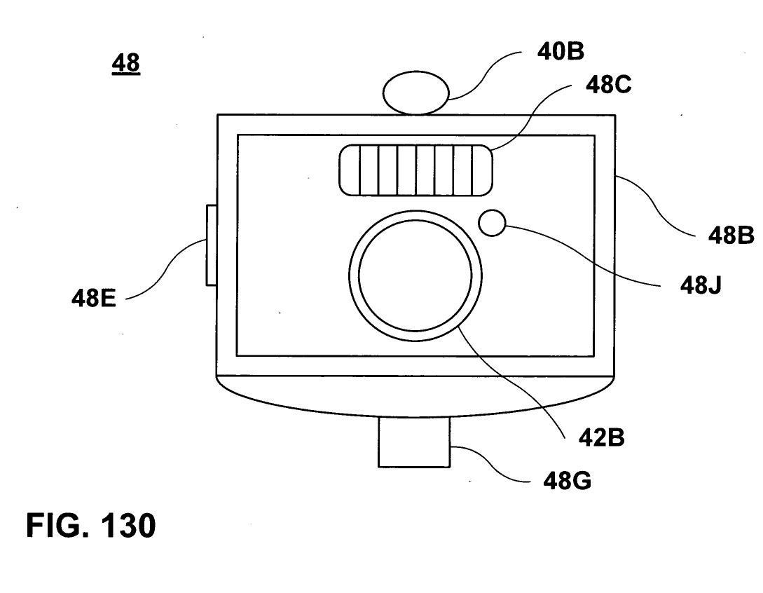

An alternative embodiment is shown in FIG. 129 in which a distance sensor 48J in the face of the VEMR™ device 48 senses the distance to the wound 52 or dermatological condition 52A and software in the VEMR™ device 48 or the diagnostic means 16A to which it is attached electronically overlays the appropriate scale ruler 76 upon the image 44G. A front view of the VEMR™ device 48 showing the distance sensor 48J is shown in FIG. 130.

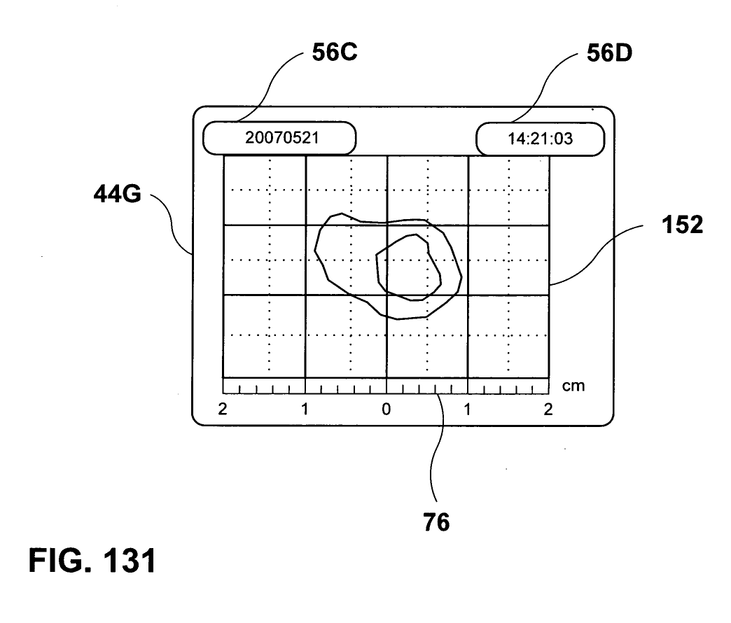

In an additional embodiment software in the VEMR™ device 48 or the diagnostic means 16A to which it is attached electronically overlays a grid 152 upon the image 44G, as shown in FIG. 131.

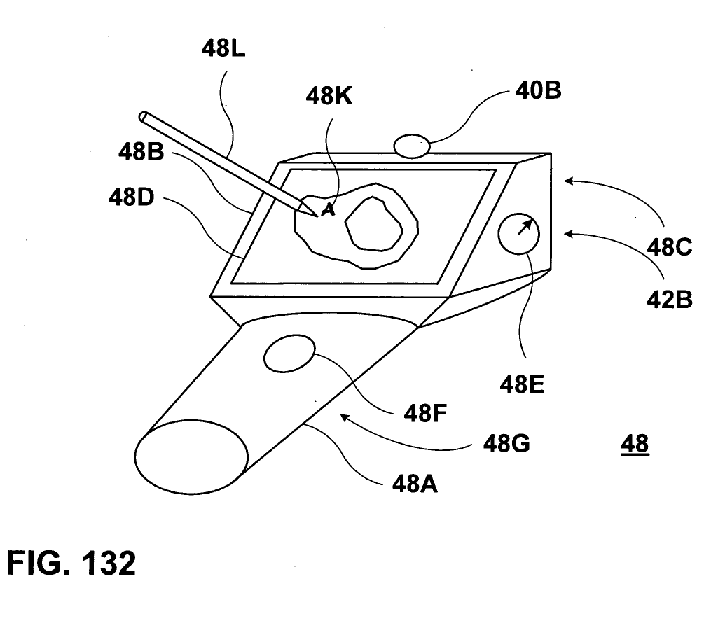

In an embodiment of the VEMR™ device 48 shown in FIG. 132 the healthcare provider 20C may annotate 48K the image 44G on the viewing screen 48D using a stylus 48L, which annotation 48K is captured on the image 44G as stored in the patient's 12A Electronic Medical Record (EMR) 60. The healthcare provider 20C may also add annotations 48K to the captured image 44G in the patient's 12A EMR 60.

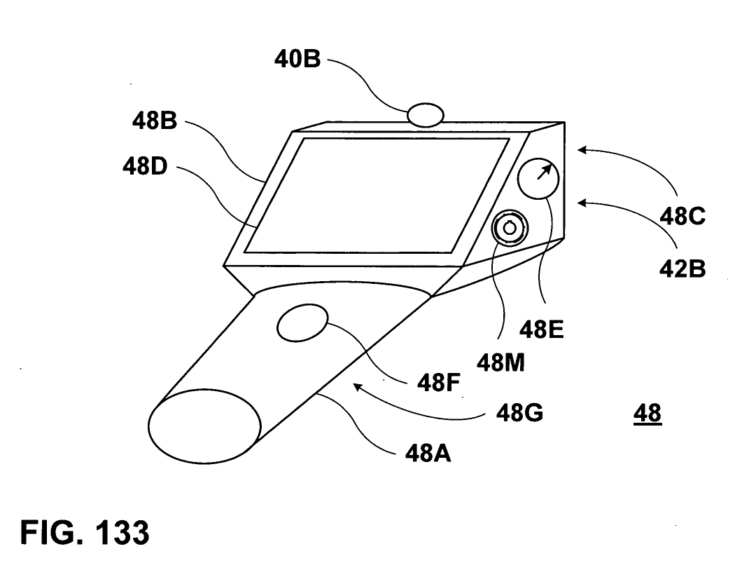

The same distance sensor 48J may be used for automatic image focus. Alternatively, FIG. 133 shows a manual focus knob 48M on the VEMR™ device 48.

### VI. Remote Diagnostic and Healthcare Monitoring Service

FIGS. 1 through 15, 26, 32, 33, 90, 102 through 104, 106 through 112, and 114 through 121 show embodiments of diagnostic means 16A. FIGS. 44, 49, 53, 57, 61, 65, 69, 73, 77, 81, 85, 86 and 113 show embodiments of data devices 80 that may be utilized in conjunction with diagnostic means 16A. FIGS. 15 through 20, 26 and 27, 107 and 114 through 116 show embodiments of a Visual Electronic Medical Record (VEMR™) device 48.

To provide remote diagnostic and health status monitoring services the remote diagnostic means 16A, VEMR™ and data devices 80 need to connect to a healthcare provider 20C as shown in FIGS. 5, 7, 11 through 15, 20, 39 through 42, 102 through 104, 106 through 110, 114 and 115. The healthcare provider 20C must have a diagnostic display and control means 24A which runs one or more software application(s) 26A. There are a variety of institutional structures and business models under which such services may be provided.

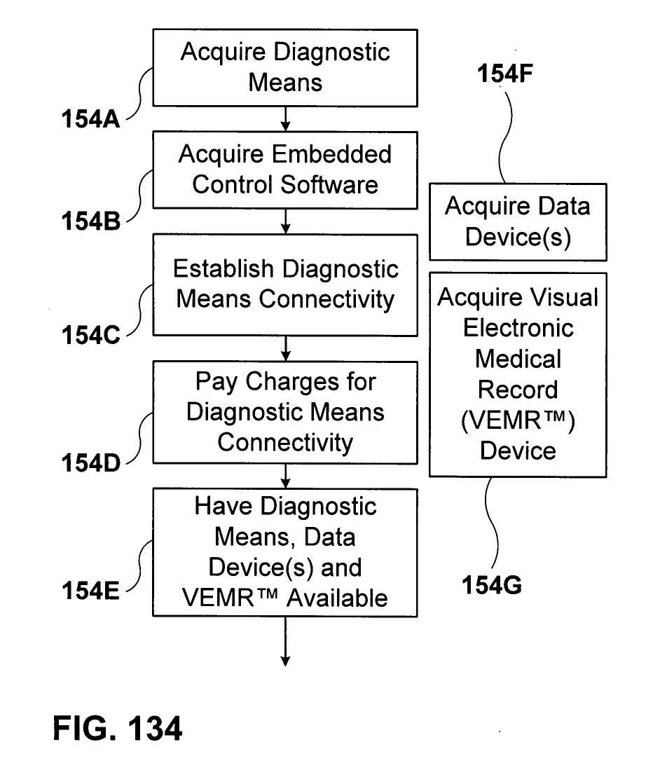

A patient or potential patient 12A must take certain steps 154 to receive remote diagnostic and health status monitoring services as shown in FIG. 134:
Acquire 154A remote diagnostic means 16A;
Acquire 154B embedded control software 18;
Establish 154C remote diagnostic means connectivity 30;
Pay charges for 154D for remote diagnostic means connectivity;
Have remote diagnostic means 16A available 154E.
   The patient or potential patient 12A may also elect to acquire 154F one or more data devices 80 and/or acquire 154G a VEMR™ device 48.

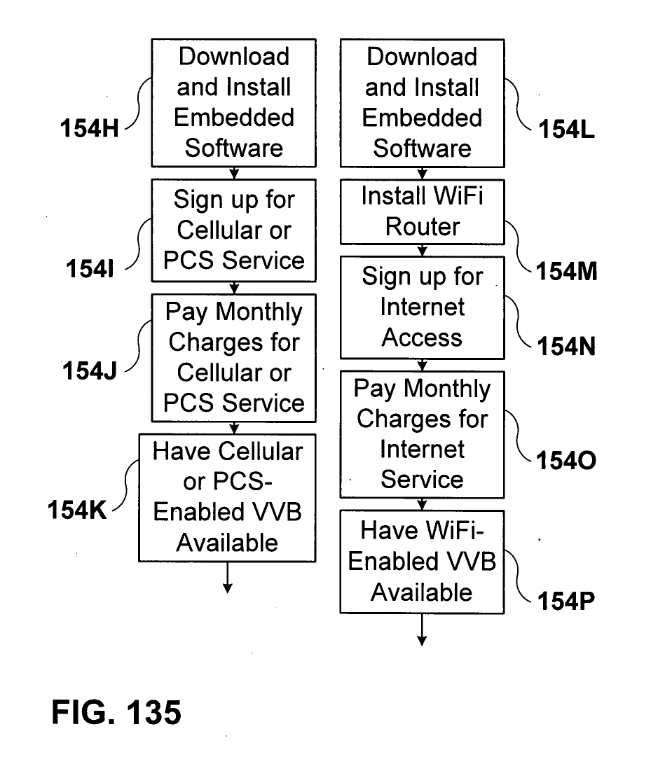

As shown in FIG. 135, if the patient 12A plans to use a Virtual Visit Book™ (VVB) 16A1 that includes a cellular or PCS wireless connection 50I as the remote diagnostic means 16A, he or she must:
Download and install 154H diagnostic means software 18A to the VVB 16A1;
Sign up for cellular or PCS wireless service 154I, including a data plan;
Pay the monthly charges for the cellular or PCS wireless service 154J; and
Have the VVB 16A1 available 154K in case of an emergency or for routine health status monitoring.
   If the patient or potential patient 12A plans to use a VVB 16A1 that includes a WiFi connection 50D as the remote diagnostic means 16A, he or she must:
Download and install 154L diagnostic means software 18A to the VVB 16A1;
Install 154M the WiFi router 156;
Sign up for Internet access 154N;
Pay the monthly charges for Internet access 154O; and
Have the VVB 16A1 available 154P in case of an emergency or for routine health status monitoring.

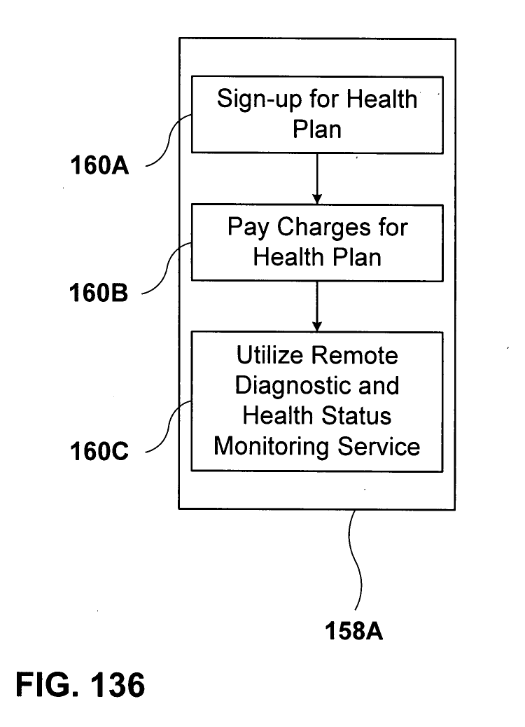

A functional block diagram 158A of a first embodiment of an institutional structure or business model under which remote diagnostic and health status monitoring services may be provided is shown in FIG. 136. In this embodiment the patient takes the steps 160 of:
Signing up 160A for health plan coverage;
Paying the fees 160B for health plan coverage;
Utilizing remote diagnostic and health status monitoring services 160C.
   In the embodiment shown in FIG. 136 the health plan makes remote diagnostics and health status monitoring available as part of its health plan. In the embodiment shown in FIG. 136 remote diagnostics and health status monitoring are provided as part of the health plan fee.

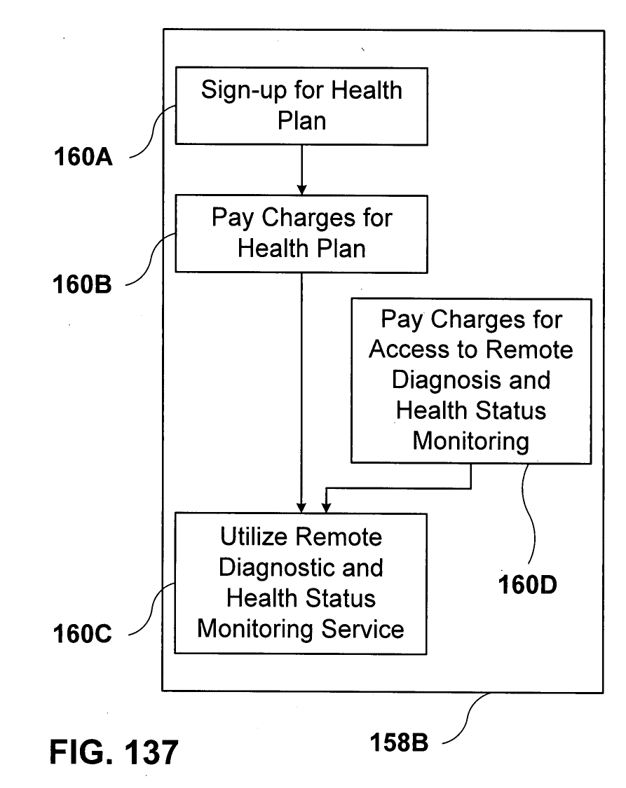

In a second embodiment of a functional block diagram of an institutional structure or business model under which remote diagnostic and health status monitoring services may be provided 158B, as shown in FIG. 137, there is a charge for using remote diagnostics and health status monitoring services 160D even though the patient 12A is covered by a health plan.

In both institutional structures or business models 158A, 158B, the health plan 160A may provide the diagnostic means 16A, data devices 80 or VEMR™ 48 to the patient 12A at no charge or for a fee.

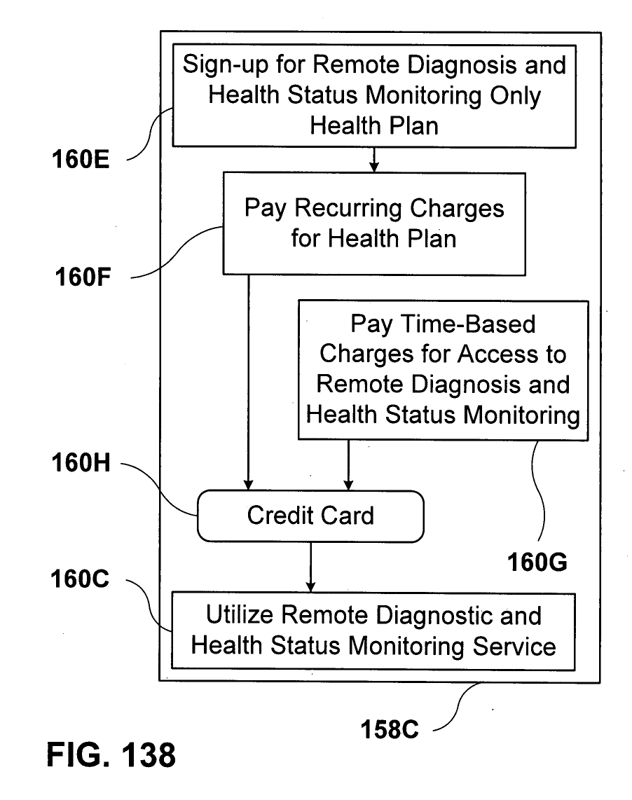

A large number of people do not have any health insurance, are under insured meaning they do not have enough medical insurance for their situation, or are self insured meaning they pay out of their pocket for health or medical services. These people, and others, may utilize a remote diagnostic and health status monitoring service if the charges are appropriate. A third embodiment of a functional block diagram of an institutional structure or business model under which remote diagnostic and health status monitoring services may be provided 158C is shown in FIG. 138. In this embodiment the patient 12A signs up for a remote diagnostic and health status monitoring only health plan 160E; pays a nominal regular fee 160F, for example, Ten Dollars ($10.00) per month; and pays a time-based fee 160G for using such remote diagnostic and health status monitoring service, for example, Two Dollars ($2.00) per minute, which fees may be paid using a credit card 160H.

### VII. Emergency Medical Services

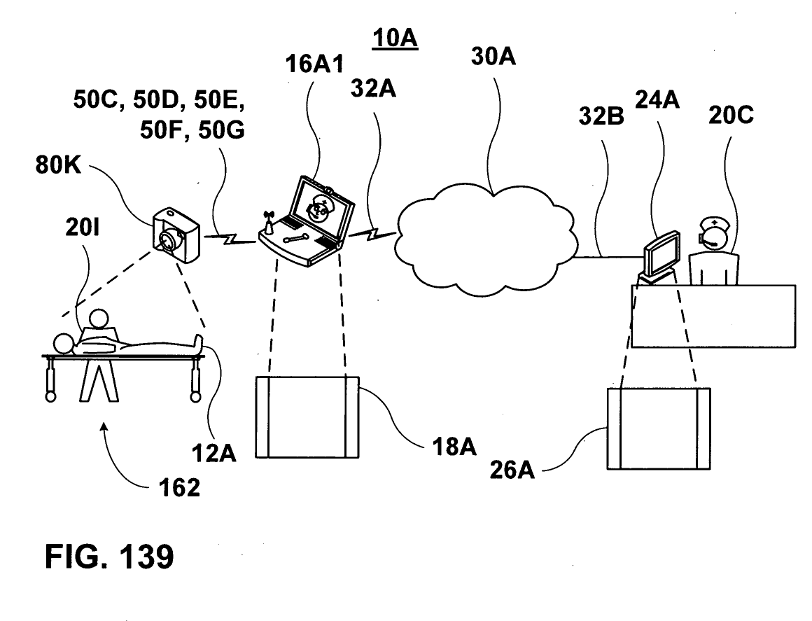

The Personal Healthcare Assistant 10A may also be used to support other health care providers 20B. For example, devices or terminals 16, data devices 80 a Visual Electronic Medical Record (VEMR™) device 48 may be deployed in ambulances and other emergency vehicles 162 as shown in FIG. 139. In the embodiment shown in FIG. 139 an Emergency Medical Technician (EMT), also sometimes known as a paramedic, 20I consults with a remote health care provider 20C about the patient's 12A condition.

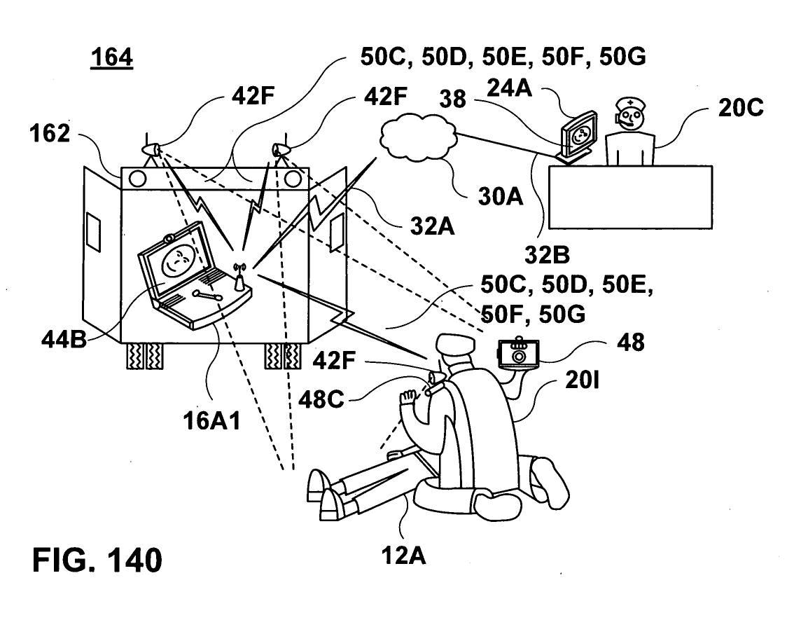

A preferred embodiment of the Mobile Diagnostic & Treatment System 164 is shown in FIG. 140. The present embodiment comprises a system 164 that includes one or more wireless cameras 42F that transmit images 44H, including a picture 44H1, streaming video 44H2, video clips 44H3, MMS 44H4, of a patient 12A as well as the treatment they are receiving from an EMT 20I to a Virtual Visit Book™ (VVB) 16A1 located inside an ambulance or other emergency vehicle 162. In addition to being displayed on the VVB 16A1, the image 44H is transmitted 32A to a remote health care provider 20C. FIG. 140 shows a wireless cameras 42F mounted on the EMT's 20I shoulder for closer viewing of a patient 12A and his or her treatment. An illumination source 48C is also mounted on the EMT's 20I shoulder to improve illumination. The EMT 20I is also using a VEMR™ 48.

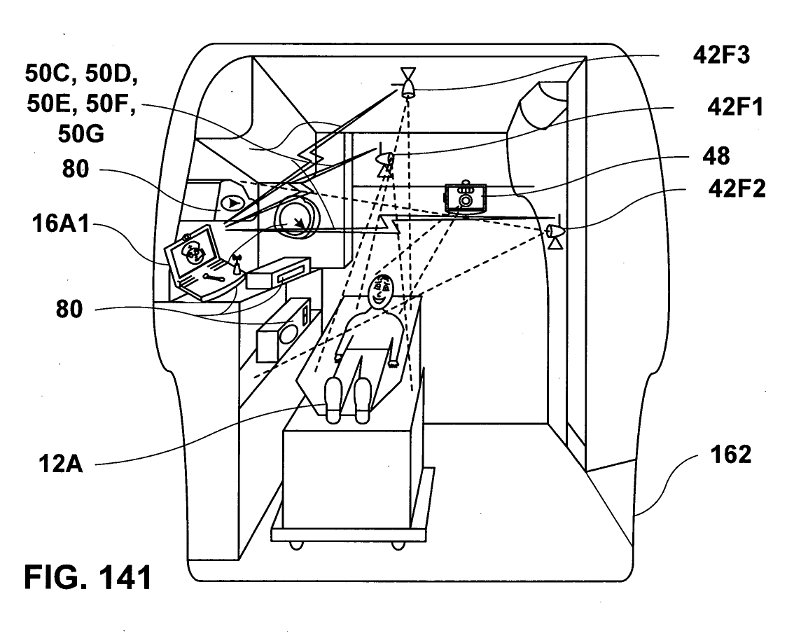

Additional elements of the preferred embodiment shown in FIG. 140 are shown in FIG. 141, which shows three additional wireless cameras 42F mounted inside the ambulance or other emergency vehicle 162, one 42F1 mounted to provide an overview of the patient 12A inside the ambulance or emergency vehicle 162. A second wireless camera 42F2 is mounted to observe readings on medical condition devices 80. A third wireless camera 42F3 is mounted in the ceiling to see the patient's face and particularly his or her eyes. These images 44H are likewise transmitted 32A to a remote health care provider 20C.

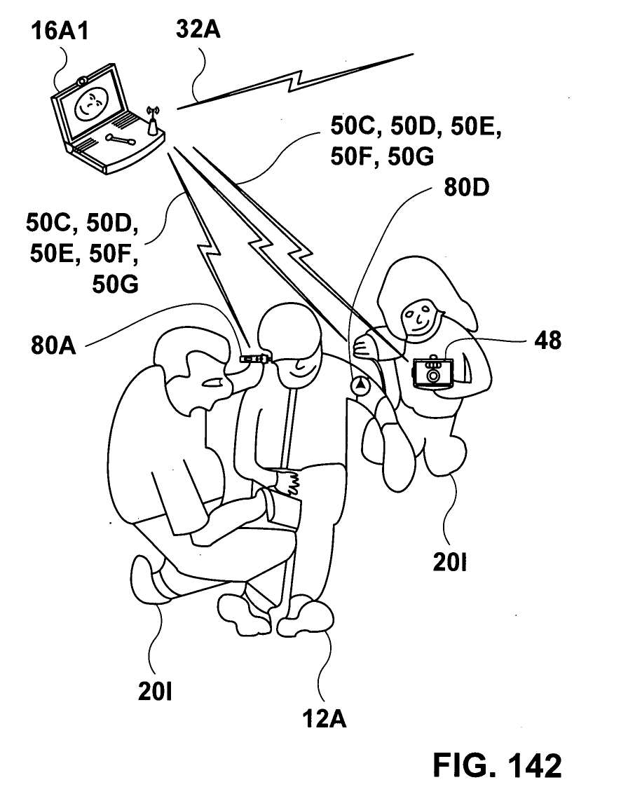

An EMT 20I may utilize a number of portable medical data devices 80 to assess a patient's 12A condition both inside and outside of an ambulance or emergency vehicle 162. FIG. 142 shows two EMTs 20I collecting medical condition data 86 from a patient 12A outside the ambulance or other emergency vehicle 162. Manufacturers are now beginning to manufacture medical condition devices 80 with built in wireless transmission capabilities, including Bluetooth 50C, Wi-Fi 50D, UWB 50E, ZigBee 50F, WiMax 50G and others. FIG. 142 shows an EMT 20I using a thermometer 80A and another EMT 20I using a blood pressure cuff 80D and a VEMR™ 48 on a patient 12A. The thermometer 80A, blood pressure cuff 80D and VEMR™ 48 transmit 50C, 50D, 50E, 50F, 50G their readings to a VVB 16A1. The remote health care provider 20C can see this data 86 on his or her display 24A.

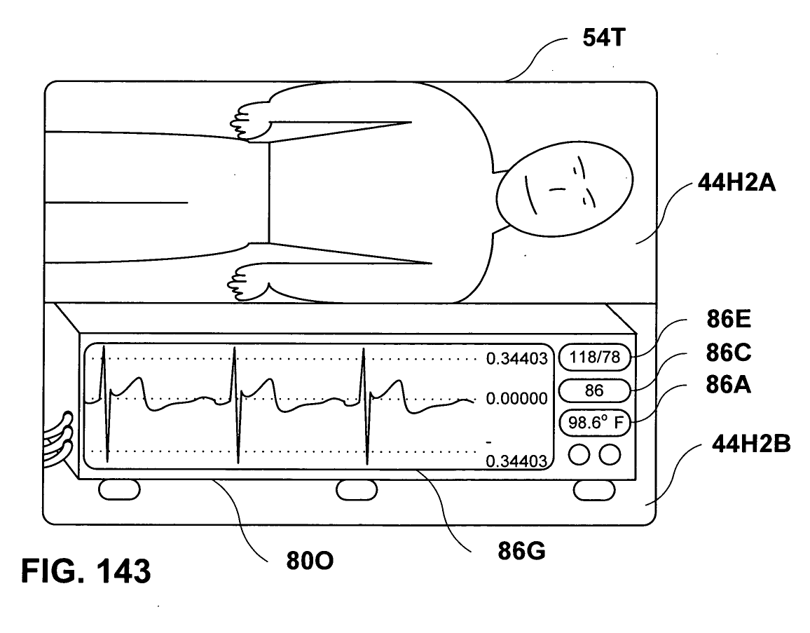

The health care provider 20C may elect to view any one or all of the wireless cameras 42F deployed in or about the ambulance or other emergency vehicle 162. FIG. 143 shows an alternative medical practitioner terminal display screen 54T on which are shown wireless camera video images 44H2. Image 44H2A is of the patient's 12A face inside the ambulance or other emergency vehicle 162 and image 44H2B is of the face of a heart monitoring device 80O showing the EKG trace 86G and other medical vital sign data.

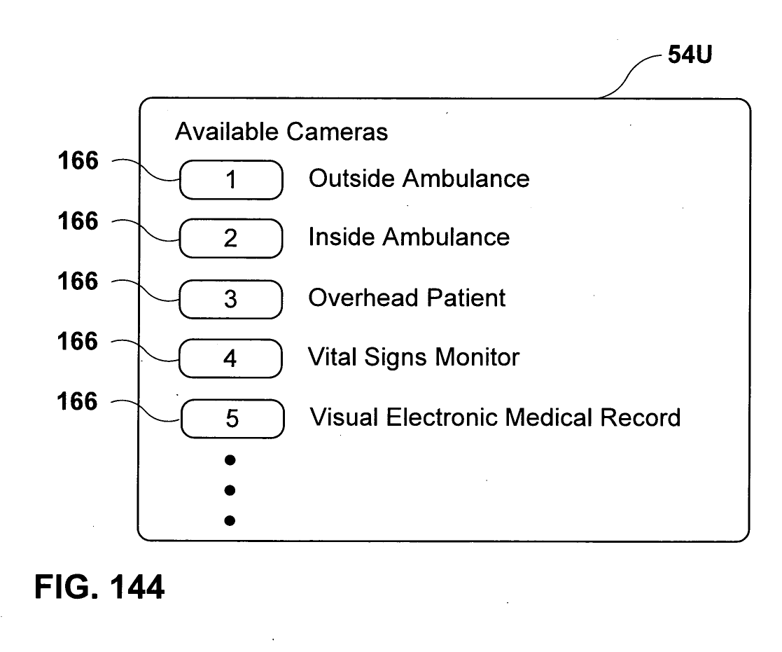

A further embodiment allows the health care provider 20C to adjust and control the various wireless cameras 42F and the VEMR™ 48. FIG. 144 shows a health care provider's 20C diagnostic, display and control means 24A screen 54U that allows the health care provider 20C to select a particular camera 42F or a VEMR™ 48 by clicking a touch screen soft button 170 on the screen 54U.

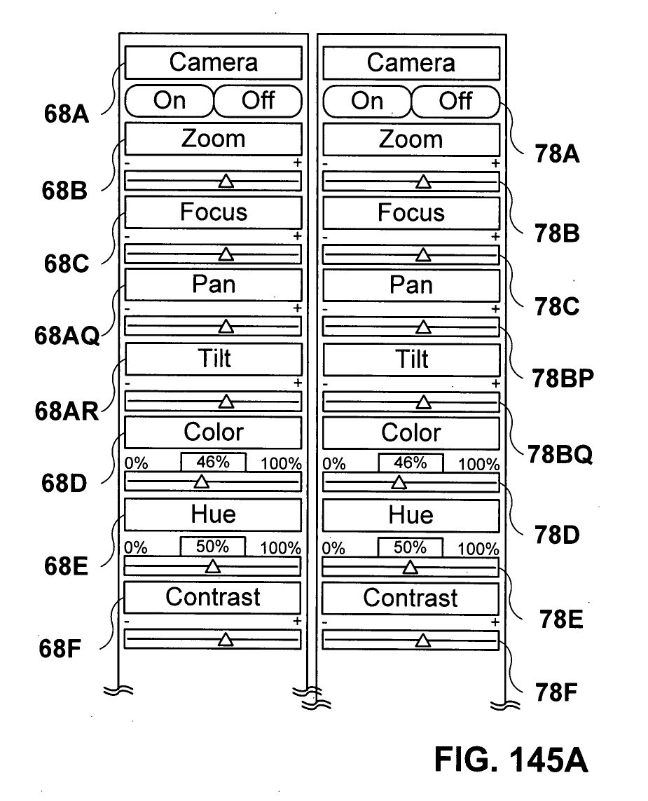

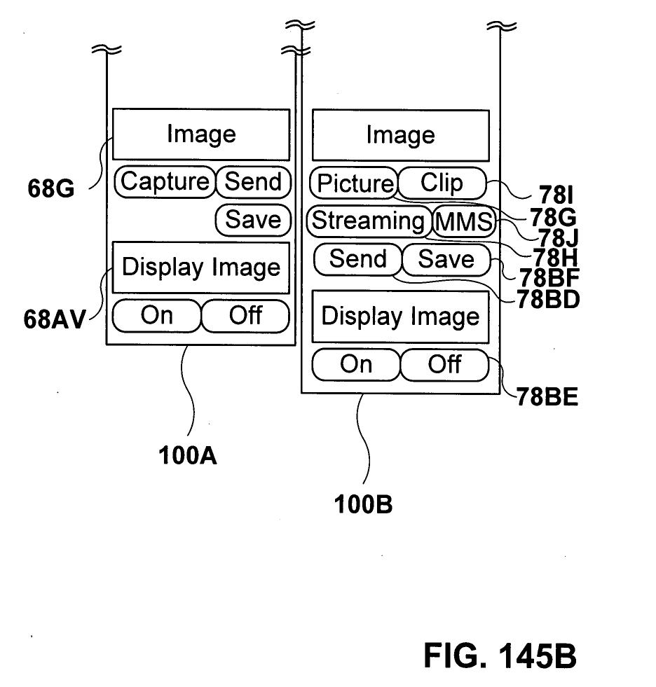

FIG. 145 shows a thirteenth embodiment of a functional block diagram 100 of embedded software 18A that enables remote functional control of wireless cameras 42F for the diagnostic means 16A as well as a software application 26A that enables remote functional control of the wireless cameras 42F connected to the diagnostic means 16A. The functions shown in 100A are deployed as a component of 18A, the software embedded in a diagnostic means 16A; those in 100B as a component of the diagnostic, display and control software application 26A.

The functions 68 embedded in a diagnostic device 16A for controlling the wireless cameras 42F include, but are not limited to:
Turn the camera 42F on or off 68A;
Zoom 68B the camera 42F;
Focus 68C the camera 42F;
Pan 68AS camera 42F;
Tilt 68AT camera 42F;
Adjust the camera 42F color 68D;
Adjust the camera 42F hue 68E;
Adjust the camera 42F contrast 68F;
Turn illumination 48C on or off 68J;
Adjust the illumination 68K;
Capture an image 68G; and
Turn the camera 42F display 68AV on or off.

The functional control means 78 in the diagnostic, display and control software application 26A for remotely controlling a wireless camera 42F via a diagnostic device 16A include, but are not limited to:
An “off” button and an “on” button 78A for controlling 68A the camera 42F;
A slider 78B that adjusts the zoom 68B from zero percent (0%) to one hundred percent (84%);
A slider 78C that adjusts the camera focus 68C from minus (−) to plus
A slider 78BP that adjusts the camera pan 68AQ from minus (−) to plus (+);
A slider 78BQ that adjusts the camera tilt 68AR from minus (−) to plus
A slider 78D that adjusts the color 68D from zero percent (0%) to one hundred percent (84%);
A slider 78E that adjusts the hue 68E from zero percent (0%) to one hundred percent (84%);
A slider 78F that adjusts the contrast 68F from minus (−) to plus (+);
An “off” button and an “on” button 78M for controlling 68F illumination 48C;
A slider 78N that adjusts the illumination 48C from zero percent (0%) to one hundred percent (100%);
A button 78G for capturing 68G the image 44H as a picture 44H1;
A button 78H for capturing 68G the image 44H as streaming video 44H2;
A button 78I for capturing 68G the image 44H as a video clip 44HB3;
A button 78J for capturing 68G the image 44H as a multi-media message (MMS) 44H4;
A button 78BE for sending 68G the image 44H; and
An “off” button and an “on” button 78BE for controlling the display 68AJ of the camera 80H.

An additional functional control means 78BF allows the healthcare provider 20C to save the image 44H to the patient's 12A Electronic Medical Record (EMR) 60. The software application 26A automatically tags the image 44H with the date 56A and current time 56B.

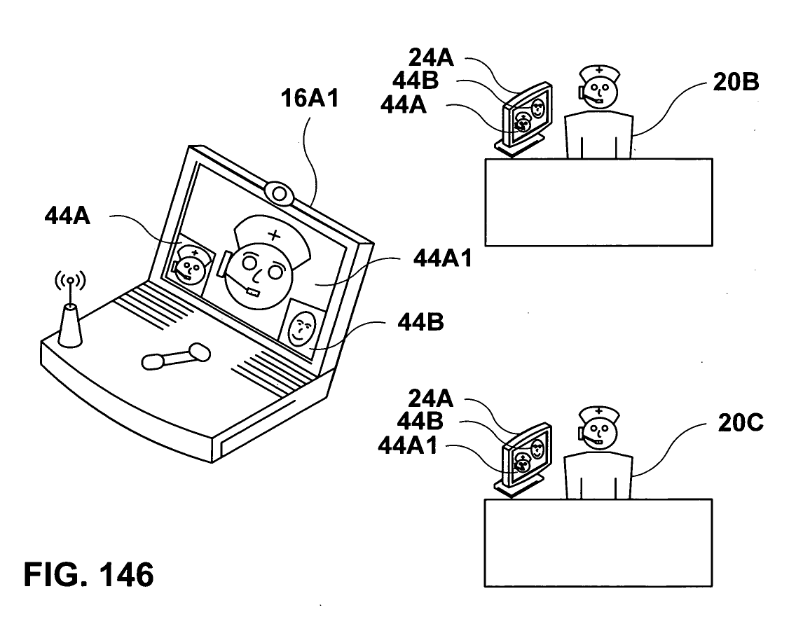

In some circumstances it may be desirable for an EMT 20T or health care provider 20C to consult with a specialist health care provider 20B with respect to handling or treating a patient 12A. In a preferred embodiment either the EMT 20I or the primary health care provider 20C can initiate a consultation. FIG. 146 shows a three-way video consultation between an EMT 20I using a VVB 16A1, a primary health care provider 20C and a specialist 20B. The EMT 20I may elect in addition to seeing the patient image 44B to see both the primary health care provider 20C image 44A as well as the specialist 20B image 44A1 on his or her VVB 16A1 display 38A, or one or the other. The primary health care provider 20C and the specialist 20B may likewise elect to see the EMT 20I, the patient 12A or the other health care provider 20C, 20B on his or her diagnostic, display and control means 24A. The specialist 20B also has the ability to view and control the wireless cameras 34C just as the primary health care provider 20C as shown in FIGS. 143 through 145.

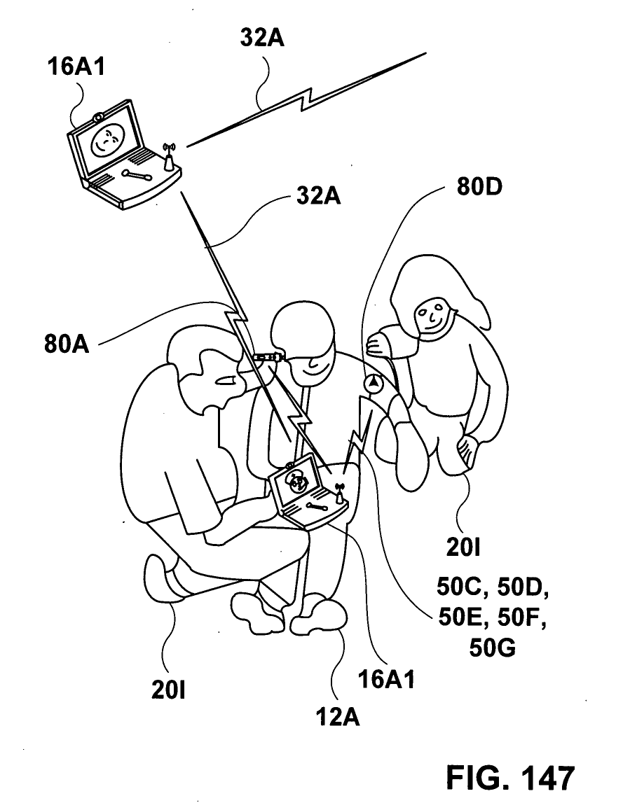

An alternative embodiment of the system 164 shown in FIG. 142 is shown in FIG. 147. In this first alternative embodiment medical condition data 86 is transmitted from a data device 80 or VEMR™ 48 to an EMT using a VVB 16A1 using a wireless technology 50C, 50D, 50E, 50F, 50G. The medical condition data 86 is then transmitted 32A along with the image 44 of the patient 12A to another VVB 16A1 via a cellular or PCS transmission 32A and then similarly transmitted 32A to the remote health care provider 20C.

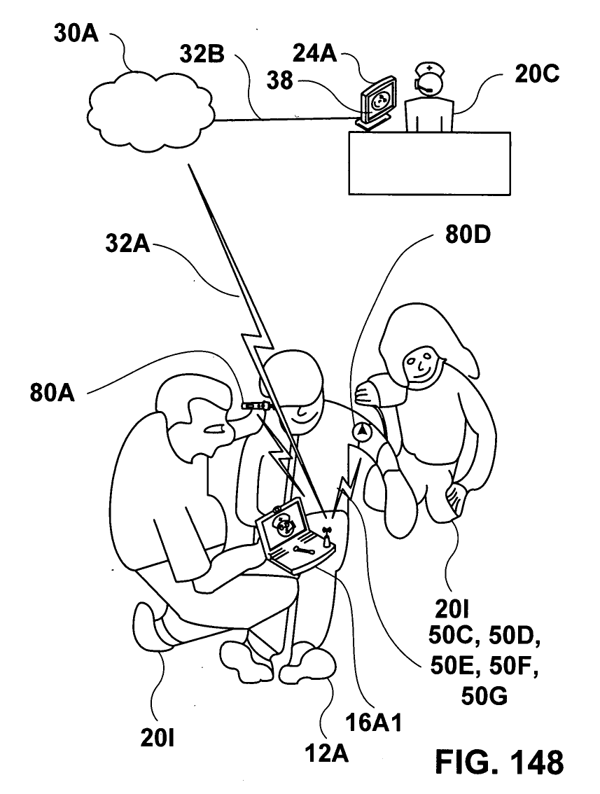

In a further system 164 embodiment shown in FIG. 148, images 44 of the patient 12A are transmitted 32A along with medical condition data 86 directly from an EMT's VVB 16A1 to a remote health care provider 20C.

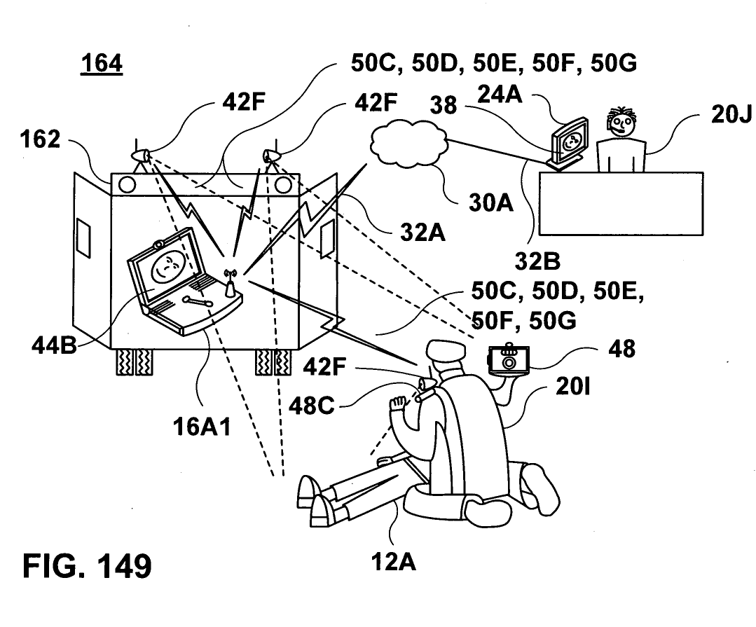

To train new EMT's 20I, emergency medical service providers currently deploy a trainer along with an ambulance 162 crew, nominally of two EMTs 20I. The Mobile Diagnostic & Treatment System 162 may be used to provide cost effective initial and recurrent training. In the preferred embodiment shown in FIG. 149 the trainer 20G remotely observes the trainee EMT 20I, and later reviews images 44 with the trainee EMT 20I as part of the training process.

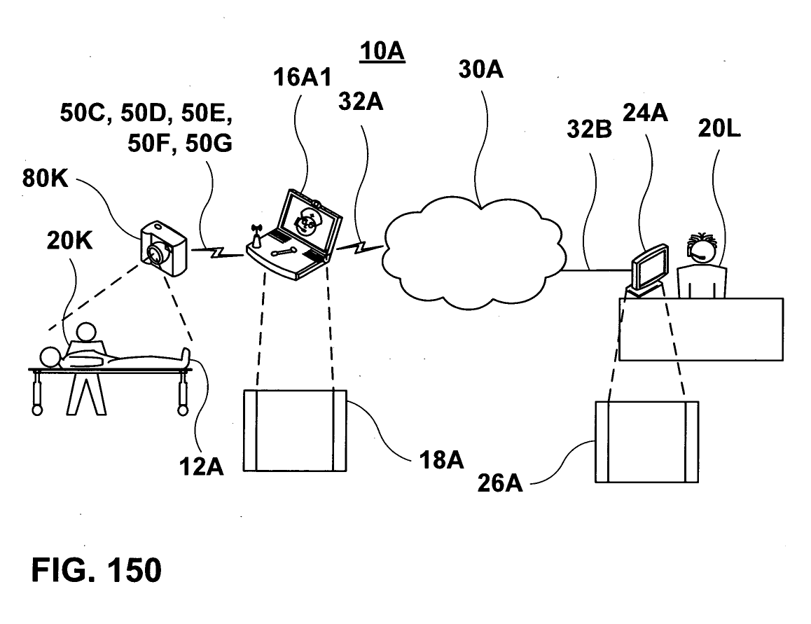

In addition to training, both Personal Healthcare Assistant 10 and the The Mobile Diagnostic & Treatment System 164 may be used for mentoring. For example, even though a professional successfully passes a licensing examination, they may require continuing supervision for a period of time. FIG. 150 shows a person being mentored 20K by a remotely located mentor 20L.

There are twelve United States national centers that stockpile medications and other supplies for use in pandemics or bioterrorism attacks. Stockpiling a few thousand Virtual Visit Books™ (VVBs) 16A1 and Visual Electronic Medical Record (VEMR™) devices 48 that can be rapidly deployed to local hospitals, public health agencies, emergency response personnel, and other healthcare facilities and personnel would enable rapid distributed mass triage and keep less critical patients from overwhelming local health facilities. Distributing these VVBs 16A1 and VEMRs™ 48 to private physicians can rapidly expand the available number of healthcare providers in such emergencies. See FIGS. 139 through 142 and 146 through 148.

### SCOPE OF THE CLAIMS

Although the present invention has been described in detail with reference to one or more preferred embodiments, persons possessing ordinary skill in the art to which this invention pertains will appreciate that various modifications and enhancements may be made without departing from the spirit and scope of the Claims that follow. The various alternatives that have been disclosed above are intended to educate the reader about preferred embodiments of the invention, and are not intended to constrain the limits of the invention or the scope of Claims.

### LIST OF REFERENCE CHARACTERS

10 Personal Healthcare Assistant
12 First person or user
12A Patient
12B Animal
12C Fire investigator
12D Policeman
12E Engineer
14 First person location
14A Nursing home
14B Medical examining room
14C Hospital or clinic patient room
16 First device or terminal
16A Remote diagnostic means
16A1 Clamshell computer
16A2 Desktop personal computer (PC)
16A3 Laptop personal computer
16A4 Ultra-mobile personal computer (UMPC)
16A5 Tablet personal computer
16A6 Cellular or Personal Communications Service (PCS) wireless phone, also known as a “cell phone:
16A7 Personal Digital Assistant (PDA) or “Smart Phone”
16A8 One Child Per Laptop (OCPL) XO personal computer
16A9 Satellite phone
16A10 Aircraft satellite phone
16A11 Walkie-talkie phone
18 Embedded software that enables remote functional control of the first person device or terminal
18A Embedded software that enables remote functional control of a remote diagnostic means
20 Second person or user
20A Call center live operator
20B Specialist or other person with particular expertise
20C Healthcare provider
20D Physical therapist or athletic coach
20E Veterinarian
20F Laboratory technician
20G Fingerprint analyst
20H Firefighter
20I Emergency Medical Technician (EMT), sometimes referred to as a paramedic
20J Trainer Emergency Medical Technician (EMT)
20K Person being mentored
20L Mentor
22 Second person location
24 Second device or terminal
24A Diagnostic, display and control means
26 Embedded software that enables assistance to a first person and enables remote functional control of the first person device or terminal
26A Diagnostic, display and control software application for remotely controlling a remote diagnostic means
28 Call center
30 Network
30A Cellular or Personal Communications Service (PCS) network
30B Satellite network
30C Public Switched Telephone Network (PSTN)
32 Connection to a network
32A Connection to a cellular or Personal Communications Service (PCS) network
32B Connection to a satellite network
32C Connection to the Public Switched Telephone Network (PSTN)
34 Third person, an assistant, a person or persons who assist in the collection of information about a first person and its conveyance to a second person
34A Nursing home worker
36 Table
38 Device or terminal display screen
38A Display screen in a clamshell computer
40 Speaker phone
40A Speaker
40B Microphone
40C External speaker
40D External microphone
40E Headset
42 Camera
42A Camera in a clamshell computer, the Virtual Visit Book™
42B Camera in a Visual Electronic Medical Record (VEMR™) device
42C Camera in an otoscope
42D Camera attached to a Personal Computer (PC)
42E Infrared camera
44 Camera image
44A Healthcare provider image
44A1 Specialist healthcare provider image
44B Patient image
44B1 Picture
44B2 Streaming video
44B3 Video clip
44B4 Multi-media message (MMS)
44C Current wound image
44C1 Picture
44C2 Streaming video
44C3 Video clip
44C4 Multi-media message (MMS)
44D Stored wound image in Electronic Medical Record (EMR)
44E Image from otoscope
44E1 Picture
44E2 Streaming video
44E3 Video clip
44E4 Multi-media message (MMS)
44F Image from ancillary camera
44F1 Picture
44F2 Streaming video
44F3 Video clip
44F4 Multi-media message (MMS)
44G Visual Electronic Medical Record (VEMR™) device image
44H Image from wireless camera
44H1 Picture
44H2 Streaming video
44H3 Video clip
44H4 Multi-media message (MMS)
46 Bandage
48 Visual Electronic Medical Record (VEMR™) device
48A Visual Electronic Medical Record (VEMR™) device handle
48B Visual Electronic Medical Record (VEMR™) device housing
48C Illumination source
48D Visual Electronic Medical Record (VEMR™) device viewing screen
48E Visual Electronic Medical Record (VEMR™) device mode of operation selector
48F Visual Electronic Medical Record (VEMR™) device “on-off” switch
48G Visual Electronic Medical Record (VEMR™) device “trigger” for capturing images or video
48H Visual Electronic Medical Record (VEMR™) device barrel or hood
48H1 Visual Electronic Medical Record (VEMR™) device barrel
48H2 Visual Electronic Medical Record (VEMR™) device hood
48H3 Visual Electronic Medical Record (VEMR™) device cone
48I Visual Electronic Medical Record (VEMR™) device adjustable distance scale
48J Visual Electronic Medical Record (VEMR™) device distance sensor
48K Visual Electronic Medical Record (VEMR™) device image annotation
48L Visual Electronic Medical Record (VEMR™) device stylus
48M Visual Electronic Medical Record (VEMR™) device manual focus knob
50 Connection means
50A Universal Serial Bus (USB)
50B FireWire connection, Institute of Electrical and Electronic Engineers (IEEE) 1394
50C Bluetooth® wireless link
50D Wireless fidelity (WiFi) wireless local area network link, Institute of Electrical and Electronic Engineers (IEEE) 802.11
50E Ultra Wide Band (UWB) connection
50F ZigBee™ wireless personal area network link, Institute of Electrical and Electronic Engineers (IEEE) 802.15.4
50G World Interoperability for Microwave Access (WiMax) connection, Institute of Electrical and Electronic Engineers (IEEE) 862.16
50H Wireless mesh network
50I Cellular or Personal Communications Service (PCS) link
50J Satellite link
50K Walkie-talkie link
52 Wound
52A Dermatological condition
54 Screen on a healthcare provider's diagnostic, display and control means
54A First screen on a healthcare provider's diagnostic, display and control means
54B Second screen on a healthcare provider's diagnostic, display and control means
54C Third screen on a healthcare provider's diagnostic, display and control means
54D Fourth screen on a healthcare provider's diagnostic, display and control means
54E Fifth screen on a healthcare provider's diagnostic, display and control means
54F Sixth screen on a healthcare provider's diagnostic, display and control means
54G Seventh screen on a healthcare provider's diagnostic, display and control means
54H Eighth screen on a healthcare provider's diagnostic, display and control means
54I Ninth screen on a healthcare provider's diagnostic, display and control means
54J Tenth screen on a healthcare provider's diagnostic, display and control means
54K Eleventh screen on a healthcare provider's diagnostic, display and control means
54L Twelfth screen on a healthcare provider's diagnostic, display and control means
54M Thirteenth screen on a healthcare provider's diagnostic, display and control means
54N Fourteenth screen on a healthcare provider's diagnostic, display and control means
54O Fifteenth screen on a healthcare provider's diagnostic, display and control means
54P Sixteenth screen on a healthcare provider's diagnostic, display and control means
54Q Seventeenth screen on a healthcare provider's diagnostic, display and control means
54R Eighteenth screen on a healthcare provider's diagnostic, display and control means
54S Nineteenth screen on a healthcare provider's diagnostic, display and control means
54T Twentieth screen on a healthcare provider's diagnostic, display and control means
54U Twenty-first screen on a healthcare provider's diagnostic, display and control means
56 Date and time stamp
56A Current date
56B Current time
56C Date of current image
56D Time of current image
56E Date of stored image
56F Time of stored image
56G Date of medical visit
56H Time of medical visit
56I Date of future medical visit
56J Time of future medical visit
56K Elapsed time
58 Measurement scale
60 Electronic Medical Record (EMR)
60A First Electronic Medical Record (EMR) screen showing major categories of an Electronic Medical Record
60B Second Electronic Medical Record (EMR) screen showing a patient's detailed information
60C Third Electronic Medical Record (EMR) screen showing a patient's medical history
60D Fourth Electronic Medical Record (EMR) screen showing a patient's current medical status
62 Categories of an Electronic Medical Record (EMR)
62A Patient identifying information
62A1 Patient name
62A2 Patient Social Security Number (SSN)
62A3 Patient date of birth
62B Detailed patient information
62B1 Patient address
62B2 Patient telephone number
62B3 Patient alternate telephone number
62B4 Patient emergency contact
62B5 Patient primary physician
62B6 Patient health plan
62B7 Patient health plan group number
62B8 Patient health plan identification number
62B9 Description of patient health plan coverage
62C Patient medical history
62C1 Type of patient medical visit
62C2 Healthcare provider for patient medical visit
62C3 Description of medical services provided to patient during visit
62C4 Audio record of patient medical visit
62C5 Image or video record of patient medical visit
62C6 Follow-up to patient medical visit
62D Patient current status
62D1 Patient currently being actively monitored
62D2 Type of scheduled medical visit
62D3 Name of the scheduled healthcare provider
62D4 Description of the medical services to be provided
62D5 Whether an audio record of the medical visit is desired
62D6 Whether an image of the medical visit is desired
62D7 Follow-up instructions, if any.
64 Electronic Medical Record (EMR) computer terminal
66 Functional block diagram of embedded software that enables remote functional control of the diagnostic means
66A Functional block diagram of embedded software that enables remote functional control of a Virtual Visit Book™ (VVB)
66B Functional block diagram of embedded software that enables remote functional control of a Visual Electronic Medical Record (VEMR™) device
68 Functions to be controlled remotely
68A Turn a camera on or off
68B Change a camera image size
68C Adjust a camera focus
68D Adjust a camera color
68E Adjust a camera hue
68F Adjust a camera contrast
68G Capture an image
68H Turn a speaker phone on or off
68I Record the Virtual Visit
68J Enter a telephone number to which to send an image or audio recording
68K Enter an electronic mail address to which to send an image or audio recording
68L Turn illumination on or off
68M Adjust illumination
68N Insert a measurement scale into an image
68O Turn a thermometer on or off
68P Capture and send a temperature reading
68Q Turn the temperature reading display of the cellular and PCS wireless phone on or off
68R Turn a stethoscope or high fidelity microphone on or off
68S Adjust the volume of a stethoscope or high fidelity microphone
68T Adjust the tone of a stethoscope or high fidelity microphone using an equalizer
68U Capture and send heart and lung sounds
68V Capture and send a pulse reading
68W Turn a scale on or off
68X Capture and send a weight reading
68Y Turn a weight reading display on or off
68Z Inflate or deflate a blood pressure cuff
68AA Capture and send a blood pressure reading
68AB Turn the blood pressure and pulse readings display on or off
68AC Turn an oximeter on or off
68AD Capture and send a reading of the percent of hemoglobin that is saturated with oxygen
68AE Turn a reading of the percent of hemoglobin that is saturated with oxygen and pulse reading display on or off
68AF Turn an electrocardiogram unit on or off
68AG Capture and send an electrocardiogram reading
68AH Turn the electrocardiogram reading display on or off.
68AI Turn a glucose meter on or off
68AJ Capture and send a glucose reading
68AK Turn a glucose reading display on or off
68AL Turn the otoscope camera display on or off
68AM Turn an ultrasound unit on or off
68AN Capture and send an ultrasound reading
68AO Turn the ultrasound display on or off
68AP Turn a spirometer on or off
68AQ Capture and send a spirometer reading
68AR Turn the spirometer display on or off
68AS Pan an external camera
68AT Tilt an external camera
68AU Turn the ancillary camera display on or off
68AV Turn a wireless camera display on or off
70 Audio recording
70A1 Audio component of video
72 Telephone number to which to send an image or audio recording
74 Electronic mail address to which to send an image or audio
76 Functional block diagram of a software application that enables remote functional control of the diagnostic means
76A Functional block diagram of a software application that enables remote functional control of a Virtual Visit Book™ (VVB)
76B Functional block diagram of a software application that enables remote functional control of a Visual Electronic Medical Record (VEMR™) device
78 Functional control means
78A “Off” button and an “on” button for controlling a camera
78B Slider that “zooms” a camera image size from zero percent (0%) to one hundred percent (100%)
78C Slider that adjusts a camera focus from minus (−) to plus (+)
78D Slider that adjusts the color from zero percent (0%) to one hundred percent (100%)
78E Slider that adjusts the hue from zero percent (0%) to one hundred percent (100%)
78F Slider that adjusts the contrast from minus (−) to plus (+)
78G Button for capturing an image as a picture
78H Button for capturing an image as streaming video
78I Button for capturing an image as a video clip
78J Button for capturing an image as a multi-media message (MMS)
78K “Off” button and an “on” button for controlling a speaker phone
78L Button for capturing an audio recording
78M Button for dialing a phone number to which an image or audio recording is to be sent
78N Button for sending an image or audio recording to an electronic mail address
78O “Off” button and an “on” button for controlling illumination
78P Slider that adjusts the illumination from zero percent (0%) to one hundred percent (100%)
78Q Button for inserting a measurement scale into an image
78R “Off” button and an “on” button for controlling a thermometer
78S Button for capturing or recapturing a temperature reading
78T Button for sending a temperature reading to a healthcare provider
78U “Off” button and an “on” button for controlling the display of a temperature reading on a diagnostic device
78V Save a temperature reading to a patient's or potential patient's Electronic Medical Record (EMR).
78W “Off” button and an “on” button for controlling a stethoscope or high fidelity microphone
78X Slider that adjusts the volume of a stethoscope or high fidelity microphone from minus (−) to plus (+)
78Y Multiple sliders that adjust the tone of a stethoscope or high fidelity microphone from minus (−) to plus (+)
78Z Button for capturing heart and lung sounds
78AA Button for sending heart and lung sounds to a healthcare provider
78AB Button for capturing or recapturing the pulse reading
78AC Button for sending the pulse reading from a stethoscope or high fidelity microphone to a healthcare provider
78AD Save the heart and lung sounds from a stethoscope or high fidelity microphone to the patient's Electronic Medical Record (EMR)
78AE Save the pulse reading from a stethoscope or high fidelity microphone to the patient's Electronic Medical Record (EMR)
78AF “Off” button and an “on” button for controlling a scale
78AG Button for capturing or recapturing a weight reading
78AH Button for sending a weight reading to a healthcare provider
78AI “Off” button and an “on” button for controlling the display of a weight reading
78AJ Save a weight reading to a patient's Electronic Medical Record (EMR)
78AK Button for inflating and deflating a blood pressure cuff
78AL Button for capturing or recapturing a blood pressure reading
78AM Button for sending the blood pressure reading to a healthcare provider
78AN “Off” button and an “on” button for controlling the display of blood pressure and pulse readings
78AO Save a blood pressure reading to a patient's Electronic Medical Record (EMR)
78AP “Off” button and an “on” button for controlling an oximeter
78AQ Button for capturing or recapturing a reading of the percent of hemoglobin that is saturated with oxygen
78AR Button for sending a reading of the percent of hemoglobin that is saturated with oxygen to a healthcare provider
78AS “Off” button and an “on” button for controlling a display of the reading of the percent of hemoglobin that is saturated with oxygen and pulse readings
78AT Save a reading of the percent of hemoglobin that is saturated with oxygen to the patient's Electronic Medical Record (EMR)
78AU “Off” button and an “on” button for controlling an electrocardiogram unit
78AV Button for capturing or recapturing an electrocardiogram reading
78AW Button for sending an electrocardiogram reading to a healthcare provider
78AX “Off” button and an “on” button for controlling a display of the electrocardiogram
78AY Save an electrocardiogram reading to a patient's Electronic Medical Record (EMR)
78AZ “Off” button and an “on” button for controlling a glucose meter
78BA Button for capturing or recapturing a glucose reading
78BB Button for sending a glucose reading to a healthcare provider
78BC “Off” button and an “on” button for controlling the display of a glucose reading
78BD Save a glucose reading to a patient's Electronic Medical Record (EMR)
78BE Button for sending the picture taken with a camera in an otoscope to a healthcare provider
78BF “Off” button and an “on” button for controlling the display of a camera in an otoscope
78BG Save the picture from a camera in an otoscope to a patient's Electronic Medical Record (EMR)
78BH “Off” button and an “on” button for controlling an ultrasound unit
78BI Button for capturing or recapturing an ultrasound readings
78BJ Button for sending ultrasound readings to a healthcare provider
78BK “Off” button and an “on” button for controlling the ultrasound display
78BL Save ultrasound readings to a patient's Electronic Medical Record (EMR)
78BM “Off” button and an “on” button for controlling a spirometer
78BN Button for capturing or recapturing spirometer readings
78BO Button for sending spirometer readings to a healthcare provider
78BP “Off” button and an “on” button for controlling the spirometer display
78BQ Save spirometer readings to a patient's Electronic Medical Record (EMR)
78BR Slider that adjusts the pan of an external camera from minus (−) to plus (+)
78BS Slider that adjusts the tilt of an external camera from minus (−) to plus (+)
80 Data device
80A Digital thermometer
80B Stethoscope or high fidelity microphone
80C Weight scale
80D Blood pressure cuff
80E Oximeter
80F Electrocardiogram (EKG)
80G Glucose meter
80H Otoscope
80I Ultrasound device
80J Spirometer
80K External camera
80L Heart rate monitor
80M Chemical sniffer
80N Fingerprint device
80O Soil sampling device
80P Heart monitoring device
82 Connection interface device
84 Functional block diagram of connection interface device
86 Data device input to connection interface device
86A Temperature reading
86B Heart and breathing sounds
86C Pulse rate reading
86D Weight reading
86E Blood pressure reading
86F Reading of the percent of hemoglobin that is saturated with oxygen
86G Electrocardiogram
86H Glucose reading
86I Otoscope reading
86J Ultrasound reading
86K Spirometer reading
88 Preamplifier in connection interface device
90 Amplifier in connection interface device
92 Connection interface in connection interface device
92A Universal Serial Bus (USB) interface in connection interface device
92B FireWire interface in connection interface device
92C Bluetooth™ interface in connection interface device
92D Wireless fidelity (WiFi) interface in connection interface device
92E Ultra Wide Band (UWB) interface in connection interface device
92F ZigBee™ interface in connection interface device
92G World Interoperability for Microwave Access (WiMax) interface in connection interface device
94 Radio system
94C Bluetooth® radio system in connection interface device
94D Wireless fidelity (WiFi) radio system in connection interface device
94E Ultra Wide Band (UWB) radio system in connection interface device
94F ZigBee™ radio system in connection interface device
94G World Interoperability for Microwave Access (WiMax) radio system in connection interface device
94H Audio interface in connection interface device
96 Antenna systems
98 Modem
100 Functional block diagram of embedded software that enables remote functional control of data devices for the diagnostic means as well as the corresponding software application that enables remote functional control of the data devices for the diagnostic means
100A Functional block diagram of software embedded in a diagnostic means that enables remote functional control of data devices via the diagnostic means
100B Software application deploy on the diagnostic, display and control means that enables remote functional control of the data devices via the diagnostic means
102 Temperature pop-up window
104 Temperature location on body
104A Temperature taken in the ear
104B Temperature taken on the ear lobe
104C Temperature taken rectally
104D Temperature taken in other location
106 “Done” button
108 Temperature window on screen displayed on a diagnostic, display and control means
110 Pulse window on screen displayed on a diagnostic, display and control means
112 Transducer for converting stethoscope sounds to electrical signals
114 Weight window on screen displayed on a diagnostic, display and control means
116 Blood pressure window on screen displayed on a diagnostic, display and control means
118 Oximeter window on screen displayed on a diagnostic, display and control means
120 Electrocardiogram window on screen displayed on a diagnostic, display and control means
122 Glucose window on screen displayed on a diagnostic, display and control means
124 Otoscope (picture) window on screen displayed on a diagnostic, display and control means
126 Ultrasound (video) window on screen displayed on a diagnostic, display and control means
128 Spirometer window on screen displayed on a diagnostic, display and control means
130 Personal Computer (PC) ports
130A Data port
130B “Line out” port
130C Audio port
132 Pool
134 Automobile
136 Global Positioning Service (GPS)
138 Prosthesis
140 Watch
140A Stop watch
142 Ship
144 Satellite
146 Satellite earth station
148 Airplane
150 Police suspect
152 Grid
154 Steps that must be taken by a patient or potential patient to receive remote diagnostic or health status monitoring services
154A Acquire remote diagnostic means
154B Acquire embedded control software for remote diagnostic means
154C Establish remote diagnostic means connectivity
154D Pay charges for remote diagnostic means connectivity
154E Have remote diagnostic means available
154F Acquire one or more data devices
154G Acquire a Visual Electronic Medical Record (VEMR™) device
154H Download and install diagnostic means software to a cellular or Personal Communications Service (PCS)-enabled Virtual Visit Book (VVB)
154I Sign up for cellular or Personal Communications Service (PCS) wireless service
154J Pay the monthly charges for the cellular or Personal Communications Service (PCS) wireless service
154K Have the cellular or Personal Communications Service (PCS)-enabled Virtual Visit Book™ (VVB), data devices or Visual Electronic Medical Record (VEMR™) device available in case of an emergency or for routine health status monitoring
154L Download and install diagnostic means software to a Wireless Fidelity (WiFi)-enabled Virtual Visit Book™ (VVB)
154M Install a Wireless Fidelity (WiFi) router
154N Sign up for Internet access
154O Pay the monthly charges for Internet access
154P Have the Wireless Fidelity (WiFi)-enabled Virtual Visit Book™ (VVB), data devices or Visual Electronic Medical Record (VEMR™) device available in case of an emergency or for routine health status monitoring
156 Wireless router
158 Functional block diagram of an institutional structure or business model under which remote diagnostic and health status monitoring services may be provided
158A Functional block diagram of an institutional structure or business model under which remote diagnostic and health status monitoring services are provided as part of a health insurance plan
158B Functional block diagram of an institutional structure or business model under which remote diagnostic and health status monitoring services are provided for an additional fee as part of a health insurance plan
158C Functional block diagram of an institutional structure or business model under which remote diagnostic and health status monitoring services are provided for a recurring fee and a time-based utilization fee
160 Steps a patient or potential patient takes to get remote diagnostic and health status monitoring services
160A Sign up for health plan coverage
160B Pay the fees for health plan coverage
160C Utilize remote diagnostic and health status monitoring services
160D Charge for using remote diagnostics and health status monitoring services
160E Sign up for a remote diagnostic and health status monitoring only health plan
160F Pays a recurring fee
160G Pay a time-based fee for using such remote diagnostic and health status monitoring service
160H Credit card payment
162 Ambulance
164 Mobile Diagnostic & Treatment System
166 Soft button

What is claimed is:

1. An apparatus comprising:
a housing;
a transceiver; said transceiver being contained in said housing;
said housing including a camera (34A), a display (40A), a speaker (36A), and a microphone (36B); said camera (34A), said display (40A), said speaker (36A) and said microphone (36B) being connected to said transceiver;
said transceiver for providing healthcare for a patient (12A); said patient (12A) generally using said transceiver in a location which is remote from a healthcare facility;
said healthcare facility being staffed by a healthcare provider;
said transceiver for conveying information concerning said patient to said healthcare provider;
said healthcare provider for providing advice concerning said patient and for partially controlling said transceiver.

2. An apparatus as recited in claim 1, in which said transceiver is connected to a network using a wired connection.

3. An apparatus as recited in claim 1, in which said transceiver is connected to a network using a wireless connection.

4. An apparatus as recited in claim 3, in which said wireless connection is established using a cellular telephone radio frequency.

5. An apparatus as recited in claim 3, in which said wireless connection is established using a Wi-Fi radio frequency.

6. An apparatus as recited in claim 3, in which said wireless connection is established using a WiMax telephone radio frequency.

7. An apparatus as recited in claim 1, in which said transceiver further includes a diagnostic software program (18A).

8. An apparatus as recited in claim 1, in which said transceiver is used to provide triage for a patient (12A).

9. An apparatus as recited in claim 1, in which said transceiver is automatically activated when said housing is opened.

10. An apparatus as recited in claim 1, in which said transceiver is used for health monitoring.

11. An apparatus as recited in claim 1, further comprising a data device (94).

12. An apparatus as recited in claim 11, in which said data device (94) is connected to said transceiver by a wireless connection (30A).

13. An apparatus as recited in claim 12, in which said wireless connection (30A) is a Bluetooth® connection (84A).

14. An apparatus as recited in claim 12, in which said wireless connection (30A) is a Wi-Fi connection (84B).

15. An apparatus as recited in claim 12, in which said wireless connection (30A) is an ultra-wide band connection (84C).

16. An apparatus as recited in claim 11, in which said data device (94) is connected to said transceiver by a wired connection (30B).

17. An apparatus as recited in claim 16, in which said wired connection (30B) uses a USB connector (84D).

18. An apparatus as recited in claim 16, in which said wired connection (30B) uses a FireWire connector (84E).

19. An apparatus as recited in claim 11, in which said data device (94) is controlled remotely by said health care provider.

20. An apparatus as recited in claim 11, in which said data device (94) is a thermometer (94A).

21. An apparatus as recited in claim 11, in which said data device (94) is an acoustic sensor (94B).

22. An apparatus as recited in claim 11, in which said data device (94) is a weight measurement device (94C).

23. An apparatus as recited in claim 11, in which said data device (94) is a blood pressure measurement device (94D).

24. An apparatus as recited in claim 11, in which said data device (94) is a device (94E) which measures the level of oxygen in the blood.

25. An apparatus as recited in claim 11, in which said data device (94) is a device for obtaining an electrocardiograph (94F).

26. An apparatus as recited in claim 11, in which said data device (94) is a glucose measurement device (94G).

27. An apparatus as recited in claim 11, in which said data device (94) is an otoscope (94H).

28. An apparatus as recited in claim 11, in which said data device (94) is an ultrasound imaging device (94I).

29. An apparatus as recited in claim 11, in which said data device (94) is an external camera (94J).

30. An apparatus as recited in claim 1, in which said transceiver is used for conveying information in a veterinary practice.

31. A method comprising the steps of:
using a healthcare transceiver means (16A) for collecting a set of data;
said healthcare transceiver means (16A) for collecting said set of data including an embedded software application (18); and
conveying said set of data from said healthcare transceiver means (16A) to a healthcare display and control means (24);
said healthcare display and control means (24) including a software application (26).

32. A method as recited in claim 31, further comprising the step of:
controlling said healthcare transceiver means (16A) using said healthcare display and control means (24).

33. A method comprising the steps of:
providing a website; said website including a database of medical information;
offering a service which furnishes a user (12) with an access code; said access code enabling said user to utilize said database of medical information on said website;
calling a call center (28); said call center (28) being staffed by a health care provider (20C); and
supplying interactive advice to said user from said call center (28).

34. A method as recited in claim 33, further comprising the step of:
using a healthcare transceiver means to provide information to said call center (28); said transceiver including a microphone (36B), a speaker (36A) and a camera (34).

35. A method as recited in claim 33, further comprising the step of:
providing data to said call center (28) using a data device (94); said data device (94) being connected to said transceiver.

36. A method as recited in claim 33, in which said data device (94) is partially controlled by said call center (28).

37. An apparatus as recited in claim 1, in which said housing is configured as a clamshell.

38. An apparatus as recited in claim 2, in which said transceiver automatically connects to said network when said housing is opened.

39. An apparatus as recited in claim 1, in which said transceiver automatically sets up a Visual Virtual Visit when said housing is opened.

40. An apparatus as recited in claim 1, in which said transceiver automatically sets up a Visual Virtual Visit when said housing is opened.

41. An apparatus as recited in claim 1, in which said transceiver is used to conduct a health check-up.

42. An apparatus as recited in claim 1, in which said transceiver is used for health status monitoring.

43. An apparatus as recited in claim 1, in which said transceiver is used to automatically connect to a 911 operator when said housing is opened.

44. An apparatus as recited in claim 1, in which said transceiver is used in a vehicle.

45. An apparatus as recited in claim 1, further including a GPS device.

46. An apparatus as recited in claim 1, in which said transceiver is used to fit a prosthetic device.

47. An apparatus as recited in claim 1, in which said transceiver is used for physical rehabilitation.

48. An apparatus as recited in claim 1, in which said transceiver is used for athletic performance coaching.

49. An apparatus as recited in claim 1, in which said transceiver is used to fingerprint a criminal suspect.

50. An apparatus as recited in claim 1, in which said transceiver is used to for soil sampling.

51. An apparatus as recited in claim 1, further comprising a Visual Electronic Medical Record device.

52. An apparatus as recited in claim 17, in which said Visual Electronic Medical Record device connects to said housing said USB port.

53. An apparatus as recited in claim 43, in which said Visual Electronic Medical Record device is used for medical imaging.

54. An apparatus as recited in claim 45, in which said Visual Electronic Medical Record device is used to create a captured image (44); said captured image (44) including a measurement scale (58) to aid in comparing a plurality of said images (44).

55. An apparatus as recited in claim 43, in which said Visual Electronic Medical Record device is connected to a computer terminal (64).

56. An apparatus as recited in claim 43, in which said Visual Electronic Medical Record device is partially controlled by said call center (28).

57. An apparatus as recited in claim 43, further comprising a fixed focus hood (48H) for monitoring a wound.

58. An apparatus as recited in claim 43, further comprising a fixed focus hood (48H) for monitoring a dermatological condition.
